SEQUENCE LISTING (184 sequences)

SEQ ID NO: 1 - protein, 574 aa, Clostriduim pasteuranum
MKTIIINGVQFNTDEDTTILKFARDNNIDISALCFLNNCNNDINKCEICTVEVEGTGLVTACDTLIEDGMIINTNSDAVNEKIKSRISQLLDIHEFKCGPCNRRENCEFLKLVIKYKARASKPFLPKDKTEYVDERSKSLTVDRTKCLLCGRCVNACGKNTETYAMKFLNKNGKTIIGAEDEKCFDDTNCLLCGQCIIACPVAALSEKSHMDRVKNALNAPEKHVIVAMAPSVRASIGELFNMGFGVDVTGKIYTALRQLGFDKIFDINFGADMTIMEEATELVQRIENNGPFPMFTSCCPGWVRQAENYYPELLNNLSSAKSPQQIFGTASKTYYPSISGLDPKNVFTVTVMPCTSKKFEADRPQMEKDGLRDIDAVITTRELAKMIKDAKIPFAKLEDSEADPAMGEYSGAGAIFGATGGVMEAALRSAKDFAENAELEDIEYKQVRGLNGIKEAEVEINNNKYNVAVINGASNLFKFMKSGMINEKQYHFIEVMACHGGCVNGGGQPHVNPKDLEKVDIKKVRASVLYNQDEHLSKRKSHENTALVKMYQNYFGKPGEGRAHEILHFKYKK

SEQ ID NO: 2 - protein, 421 aa, Desulfovibrio vulgaris
MSRTVMERIEYEMHTPDPKADPDKLHFVQIDEAKCIGCDTCSQYCPTAAIFGEMGEPHSIPHIEACINCGQCLTHCPENAIYEAQSWVPEVEKKLKDGKVKCIAMPAPAVRYALGDAFGMPVGSVTTGKMLAALQKLGFAHCWDTEFTADVTIWEEGSEFVERLTKKSDMPLPQFTSCCPGWQKYAETYYPELLPHFSTCKSPIGMNGALAKTYGAERMKYDPKQVYTVSIMPCIAKKYEGLRPELKSSGMRDIDATLTTRELAYMIKKAGIDFAKLPDGKRDSLMGESTGGATIFGVTGGVMEAALRFAYEAVTGKKPDSWDFKAVRGLDGIKEATVNVGGTDVKVAVVHGAKRFKQVCDDVKAGKSPYHFIEYMACPGGCVCGGGQPVMPGVLEAMDRTTTRLYAGLKKRLAMASANKA

SEQ ID NO: 3 - protein, 468 aa, Entamoeba histolytica
MPPKPSHTLTGHDHNHSIQFDWSKCMGCGMCATKCTFGVLVKQPPKIPPFVQPNREKLSQENTDKTRVLIDESECTGCGQCSLVCNFGSITPIDHLVDTFKAKEAGKKLVAMIAPSTRLGVAEAMGMPIGSTAMAQLVHCLRLIGFDYVFDVDAGADKTTMDDYAEVIEMKKEGKGPAITSCCPAWIELVEKEYPDLIPNVSTARSPIGCLAGCIKRGWAKDVGIAVEDLYTVGIMPCIAKKTESQRQQIHQDYDASCTSNEIAAYFKKHLPPEECKFTQEREEALAKTEDGQCDLPFRRISGGSNIFGKTGGVCETVLRVIARNAGVDWNSCTVNKEETFKHAASGSTMTNLSVDIGGTIITGAVCHGGYAIRHACELIRKGELKVDVVEMMACVGGCLGGAGQPKIPPAKKLEMDKRRVMLDILDQQTDIRAANENTDVLGWIDKHFDHQGAHQHLHTYFTPRYQN

SEQ ID NO: 4 - protein, 491 aa, Saccharomyces cerevisiae
MSALLSESDLNDFISPALACVKPTQVSGGKKDNVNMNGEYEVSTEPDQLEKVSITLSDCLACSGCITSSEEILLSSQSHSVFLKNWGKLSQQQDKFLVVSVSPQCRLSLAQYYGLTLEAADLCLMNFFQKHFQCKYMVGTEMGRIISISKTVEKIIAHKKQKENTGADRKPLLSAVCPGFLIYTEKTKPQLVPMLLNVKSPQQITGSLIRATFESLAIARESFYHLSLMPCFDKKLEASRPESLDDGIDCVITPREIVTMLQELNLDFKSFLTEDTSLYGRLSPPGWDPRVHWASNLGGTCGGYAYQYVTAVQRLHPGSQMIVLEGRNSDIVEYRLLHDDRIIAAASELSGFRNIQNLVRKLTSGSGSERKRNITALRKRRTGPKANSREMAAATAATADPYHSDYIEVNACPGACMNGGGLLNGEQNSLKRKQLVQTLNKRHGEELAMVDPLTLGPKLEEAAARPLSLEYVFAPVKQAVEKDLVSVGSTW

SEQ ID NO: 5 - protein, 436 aa, Chlorella fusca
MCCPVVASRHAGRARHVAVRAAGPTSECDCPPTPQAKLPHWQQALDELAKPKESRRLMIAQIASAVRVAIAETIGLAPGDVTIGQLVTGLRMLGFDYVFDTLFGADLTIMEEGTELLHRLQDHLEQHPNKEEPLPMFTSCCPGWVAMVEKSNPELIPYLSSCKSPQMMLGAVIKNYYAQQVGVQPSDICNVSVMPCVRKQGEADREWFNTTGAGLARDVDHVVTTAEVGKIFLERGIKLNELPESNFDNPIGEGTGGALLFGTTGGVMEAALRTVYEVVTQKPMGRVDFEEVRGLEGIKEAEITLKPGDDSPFKAFAGADGQGITLKIAVANGLGNAKKLIKSLSEGKAKYDFIEVMACPGGCIGGGGQPRSTDKQILQKRQQAMYNLDERSTIRRSHDNPFIQALYDKFLGAPNSHKAHDLLHTHYVAGGIPEEK

SEQ ID NO: 6 - protein, 574 aa, Clostridium saccharobutylicum
MINIVIDEKTIQVQENTTVIQAALANGIDIPSLCYLNECGNVGKCGVCAVEIEGKNNLALACITKVEEGMVVKTNSEKVQERVKMRVATLLDKHEFKCGPCPRRENCEFLKLVIKTKAKANKPFVVEDKSQYIDIRSKSIVIDRTKCVLCGRCEAACKTKTGTGAISICKSESGRIVQATGGKCFDDTNCLLCGQCVAACPVGALTEKTHVDRVKEALEDPNKHVIVAMAPSIRTSMGELFKLGYGVDVTGKLYASMRALGFDKVFDINFGADMTIMEEATEFIERVKNNGPFPMFTSCCPAWVRQVENYYPEFLENLSSAKSPQQIFGAASKTYYPQISGISAKDVFTVTIMPCTAKKFEADREEMYNEGIKNIDAVLTTRELAKMIKDAKINFANLEDEQADPAMGEYTGAGVIFGATGGVMEAALRTAKDFVEDKDLTDIEYTQIRGLQGIKEATVEIGGENYNVAVINGAANLAEFMNSGKILEKNYHFIEVMACPGGCVNGGGQPHVSAKEREKVDVRTVRASVLYNQDKNLEKRKSHKNTALLNMYYDYMGAPGQGKAHELLHLKYNK

SEQ ID NO: 7 - protein, 421 aa, Desulfovibrio vulgaris
MSRIEMEKIFYEDHAPDPKADPDKLFFIQIDESKCIGCDSCQQYCPTGAIFGDTGDAHKIPHEELCINCGQCLTHCPVGAIYESQSWVTEIEKKIKAKDVKVIAMPAPAVRYALGDAFGLPVGTVTTGKMFSALKELGFDHCWDNEFTADVTIWEEGTEFVQRLTKKLDKPLPQFTSCCPGWHKYVESLYPELFPHMSSCKSPIGMLGTLAKTYGADRMKYDRAKVYTVSIMPCTAKKYEGMRPQLWDSGHKDIDATIDTRELAYMIKKAKIDFTKLPDGKRDTLMGESTGGATLFGVTGGVMEAALRYAYQAVTGKKPESMDFKGVRGLQGVKEATVNVGGVDVKVAVVHGARRFHDVCELVKAGKAPWHFIEFMACPGGCVCGGGQPVMPGVLEAADRRSTRMYAGLKKRLAMASASRA

SEQ ID NO: 8 - protein, 124 aa, Desulfovibrio vulgaris
MQIVNLTRRGFLKAACVVTGGALISIRMTGKAVAAAKQLKDYMMDRINGVYGADAKFPVRASQDNVQVQKLYADFLEKPMSHKAEQLLHTHWVDRSKAIERMKAQGAYPNPRAKEFEGNTYPYE

SEQ ID NO: 9 - protein, 606 aa, Desulfovibrio vulgaris
MNAFINGKEVRCEPGRTILEAARENGHFIPTLCELADIGHAPGTCRVCLVEIWRDKEAGPQIVTSCTTPVEEGMRIFTRTPEVRRMQRLQVELLLADHDHDCAACARHGDCELQDVAQFVGLTGTRHHFPDYARSRTRDVSSPSVVRDMGKCIRCLRCVAVCRNVQGVDALVVTGNGIGTEIGLRHNRSQSASDCVGCGQCTLVCPVGALAGRDDVERVIDYLYDPEIVTVFQFAPAVRVGLGEEFGLPPGSSVEGQVPTALRLLGADVVLDTNFAADLVIMEEGTELLQRLRGGAKLPLFTSCCPGWVNFAEKHLPDILPHVSTTRSPQQCLGALAKTYLARTMNVAPERMRVVSLMPCTAKKEEAARPEFRRDGVRDVDAVLTTREFARLLRREGIDLAGLEPSPCDDPLMGRATGAAVIFGTTGGVMEAALRTVYHVLNGKELAPVELHALRGYENVREAVVPLGEGNGSVKVAVVHGLKAARQMVEAVLAGKADHVFVEVMACPGGCMDGGGQPRSKRAYNPNAQARRAALFSLDAENALRQSHNNPLIGKVYESFLGEPCSNLSHRLLHTRYGDRKSEVAYTMRDIWHEMTLGRRVRGDSD

SEQ ID NO: 10 - protein, 572 aa, Clostridium perfringens
MNKIIINDKTIEFDGDKTILDLARENGFDIPVLCELKNCGNKGQCGVCLVEQEGNDRLLRSCAIKAKDGMVIKTDSEKVLEARKERVAELLDEHEFKCGPCKRRENCEFLKLVIKTKARAHKPFVVADKSEYVDDRSKSIVLDRSKCVKCGRCVAACRTRTATNSIKFHRIDGVRLVGPEELKCFDDTNCLLCGQCIAACPVDALSEKSHIERVQEALNDPEKHVIVAMAPAVRTSMGELFKMGYGQDVTGKLYTALRELGFDKVFDINFGADMTIMEEATELIERIKNNGPFPMLTSCCPSWVREVENYFPELVENLSSAKSPQQIFGAASKTYYPQVADIDPKKVFTVTVMPCTSKKFEADRPEMENEGIRNIDAVITTRELARMIKAAKIDFAKLEDGEVDPAMGEYTGAGVIFGATGGVMEAALRTAKDFMENDNLDNVDYEAVRGLAGIKEAEVEIAGNEYKLAVVSGAANVFELVKSGKINDYHFIEVMACPGGCVNGGGQPHISAEDSDKMDIREVRASVLYNQDKNLEKRKSHQNSALLKMYESYMGKPGHGRAHELLHMKYKK

SEQ ID NO: 11 - protein, 572 aa, Clostridium perfringens
MNKIIINDKTIEFDGDKTILDLARENGFDIPVLCELKNCGNKGQCGVCLVEQEGNDRLLRSCAIKAKDGMVIKTDSEKVLEARKERVAELLDEHEFKCGPCKRRENCEFLKLVIKTKARAHKPFVVADKSEYVDDRSKSIVLDRSKCVKCGRCVAACRTRTATNSIKFHRIDGVRLVGPEELKCFDDTNCLLCGQCIAACPVDALSEKSHIERVQDALNDPEKHVIVAMAPAVRTSMGELFKMGYGQDVTGKLYTALRELGFDKVFDINFGADMTIMEEATELIERIKNNGPFPMLTSCCPSWVREVENYFPELVENLSSAKSPQQIFGAASKTYYPQVADIDPKKVFTVTVMPCTSKKFEADRPEMENEGIRNIDAVITTRELARMIKAAKIDFAKLEDGEVDPAMGEYTGAGVIFGATGGVMEAALRTAKDFMENDNLDNVDYEAVRGLAGIKEAEVEIAGNEYKLAVVSGAANVFELVKSGKINDYHFIEVMACPGGCVNGGGQPHISAEDSDKIDIREVRASVLYNQDKNLEKRKSHQNSALLKMYENYMGKPGHGRAHELLHMKYKK

SEQ ID NO: 12 - protein, 484 aa, Megasphaera elsdenii
MPEFHSRFEKIDRRVPIDEHNCAVQFDVTKCKNCTLCRRACADTQTVLDYYSLSSTGDMPICVHCGQCSSACPFGAIVEVNDVDKVKAALKDPEKIVIFQTAPAVRVGLGEAFGMDPGTFVEGKMVAALRTLGADYVFDTDFGADLTIMEEATELLHRLQSEEIPIPQFTSCCPAWVEFAETFYPDLLQHLSSTKSPISILSPVIKTYFAQQKNIDPKKIVNVCVTPCTAKKAEIRRPELSASGLFWDEPEIRDTDICITTRELAQWIQDENIDFASLEDSKFDKAFGEASGGGRIFGNSGGVMEAAIRTAYHMFTGRPAPKDFIPFEPVRGLQGVKKATVIFGHFVLHVAAISGLGNARAFIDDLIKNDAFEDYSFIEVMACPGGCIGGGGQPKVKLPQVKKVQEARTASIYKSDEETDIKASWQNPEIETLYEAFLDEPLSEMAEFTLHTYFSDKSDQLGRMKNLTPQTNPMSPKYKPPTEE

SEQ ID NO: 13 - protein, 421 aa, Desulfovibrio desulfuricans strain
MNLVEMEKIQYVDQSPDPRANPDELFFIQIDPEKCIGCDTCQEYCPTGAIFGDTGSAHSIPHEEICINCGQCLTHCPVGAIYEVQSWVRELSEKIKDPEIKVIAMPAPAVRYGLGECFGMPVGTVTTGKMLTALQMLGFDHVWDNEFTADVTIWEEGTEFVNRLTGQIDKPLPQFTSCCPGWHKYVESFYPELFPHLSSCKSPIGMMGALAKTYGPDVMKYDRSKVYTVSIMPCTAKKYEGMRADLWSSGYKDIDATIDTRELAYMIKKAGIDFAALPDGKRDTLMGDSTGGATIFGVSGGVMEAALRYAYEAVTGKKPSSWDFTMVRGLNGIKEGTVTIGDAKINVAVVHGAKRFAEVCEVIKTGKSPWHFIEFMACPGGCVCGGGQPVMPGVLEAMDRKVSRTFAGLKERLNRMSSSKA

SEQ ID NO: 14 - protein, 585 aa, Desulfovibrio fructosovorans
MSMLTITIDGKTTSVPEGSTILDAAKTLDIDIPTLCYLNLEALSINNKAASCRVCVVEVEGRRNLAPSCATPVTDNMVVKTNSLRVLNARRTVLELLLSDHPKDCLVCAKSGECELQTLAERFGIRESPYDGGEMSHYRKDISASIIRDMDKCIMCRRCETMCNTVQTCGVLSGVNRGFTAVVAPAFEMNLADTVCTNCGQCVAVCPTGALVEHEYIWEVVEALANPDKVVIVQTAPAVRAALGEDLGVAPGTSVTGKMAAALRRLGFDHVFDTDFAADLTIMEEGSEFLDRLGKHLAGDTNVKLPILTSCCPGWVKFFEHQFPDMLDVPSTAKSPQQMFGAIAKTYYADLLGIPREKLVVVSVMPCLAKKYECARPEFSVNGNPDVDIVITTRELAKLVKRMNIDFAGLPDEDFDAPLGASTGAAPIFGVTGGVIEAALRTAYELATGETLKKVDFEDVRGMDGVKKAKVKVGDNELVIGVAHGLGNARELLKPCGAGETFHAIEVMACPGGCIGGGGQPYHHGDVELLKKRTQVLYAEDAGKPLRKSHENPYIIELYEKFLGKPLSERSHQLLHTHYFKRQRL

SEQ ID NO: 15 - protein, 421 aa, Desulfovibrio fructosovorans
MSRIEMAKIFYEQTVPPPGTNLDQAYIVQVDETKCIGCDTCMGYCPTGAITGESGEPHKVVDPAACINCGQCLTHCPVAAIYETVSFVPEIEAKLKDKNVKVIAMPAPAVRYALGDPFGMPLGAVTTEHMLTGLKQLGFDNVWDNEFTADVTIWEEGSELLARITKKLDKPLPQFTSCCPGWQKYAETFYPELLPHFSSCKSPIGMMGPLAKTYGAKELGYEPKQIYTVSIMPCTAKKFEGMRPEMDASGFRDIDATINTRELAYMMKKAGIDLPKIANGKRDAVMGESTGGATIFGVSGGVMEAALRFAYQALTKKPPQSWDFKAVRGLNGIKEATINIGGTDVKVAVVNGGKNFAKVCDEVKAGKSPYHFIEFMACPGGCVMGGGQPIMPTVLESMNRTTTKFYASLKKRLALYDAQKA

SEQ ID NO: 16 - protein, 608 aa, Thermotoga maritima
MRRFFKNNLRNLSQNGETNSVRRCFALADVTVVINGRTLTVPDNLTVIEACEKAGIEIPALCHHPRLGESIGACRVCVVEVEGARNLQPACVTKVRDGMVIKTSSDRVKTARKFNLALLLSEHPNDCMTCEANGRCEFQDLIYKYDVEPIFGYGTKEGLVDRSSPAIVRDLSKCIKCQRCVRACSELQGMHIYSMVERGHRTYPGTPFDMPVYETDCIGCGQCAAFCPTGAIVENSAVKVVLEELEKKEKILVVQTAPSVRVAIGEEFGYAPGTISTGQMVAALRRLGFDYVFDTNFGADLTIMEEGSEFLERLEKGDLEDLPMFTSCCPGWVNLVEKVYPELRTRLSSAKSPQGMLSAMVKTYFAEKLGVKPEDIFHVSIMPCTAKKDEALRKQLMVNGVPAVDVVLTTRELGKLIRMKKIPFANLPEEEYDAPLGISTGAAALFGVTGGVMEAALRTAYELKTGKALPKIVFEEVRGLKGVREAEIDLDGKKIRIAVVHGTANVRNLVEKILRREVKYHFVEVMACPGGCIGGGGQPYSRDPEILRKRAEAIYTIDERMTLRKSHENPAIKKLYEEYLEHPLSHKAHELLHTYYEDRSRKKRLAVK

SEQ ID NO: 17 - protein, 645 aa, Thermotoga maritima
MKIYVDGREVIINDNERNLLEALKNVGIEIPNLCYLSEASIYGACRMCLVEINGQITTSCTLKPYEGMKVKTNTPEIYEMRRNILELILATHNRDCTTCDRNGSCKLQKYAEDFGIRKIRFEALKKEHVRDESAPVVRDTSKCILCGDCVRVCEEIQGVGVIEFAKRGFESVVTTAFDTPLIETECVLCGQCVAYCPTGALSIRNDIDKLIEALESDKIVIGMIAPAVRAAIQEEFGIDEDVAMAEKLVSFLKTIGFDKVFDVSFGADLVAYEEAHEFYERLKKGERLPQFTSCCPAWVKHAEHTYPQYLQNLSSVKSPQQALGTVIKKIYARKLGVPEEKIFLVSFMPCTAKKFEAEREEHEGIVDIVLTTRELAQLIKMSRIDINRVEPQPFDRPYGVSSQAGLGFGKAGGVFSCVLSVLNEEIGIEKVDVKSPEDGIRVAEVTLKDGTSFKGAVIYGLGKVKKFLEERKDVEIIEVMACNYGCVGGGGQPYPNDSRIREHRAKVLRDTMGIKSLLTPVENLFLMKLYEEDLKDEHTRHEILHTTYRPRRRYPEKDVEILPVPNGEKRTVKVCLGTSCYTKGSYEILKKLVDYVKENDMEGKIEVLGTFCVENCGASPNVIVDDKIIGGATFEKVLEELSKNG

SEQ ID NO: 18 - protein, 1206 aa, Nyctotherus ovalis
MISRLIAKKAPLFLRTFATSEMISLKIDGKIISVPKGIMLADAIKKAGANVPTMCYHPDLPTSGGICRVCLVESAKSPGYPIISCRTPVEEGMEIVTQGSKMKEYRQANLALMLSRHPNACLSCTSNTNCKTQELSANMNIGQCGFANATPPKNDDSYDMTTAIERDNDKCINCDICVHTCSLQGLNALGFYNEEGHAVKSMGTLDVSECIQCGQCINRCPTGAITEKSEIRPVLDAINIQQRLVFQMAPSIRVAVAEEFGIKPGEKILKNEIATALRKLGSNVFVLDTNFSADLTIIEEGHELIERLYRNVTGKKLLGGDHMPIDLPMLTSCCPGWIMFIEKNYPDLLNNLSTCKSPQGMLGALIKGYWAKNIKKMDPKDIVSVSIMPCTAKKAEKERPQLRGDEGYKDVDYILTTRELAKMLKQSNIDLAKMEPTPFDKVMSEGTGAAVIFGVTGGVMEAALRTANEVITGREVPFKNLNIEAVRGMEGIREAGIKLENVLDKYKAFEGVTVKVAIAHGPNNARKVMDIIKQAKESGKPAPWHFVEVMACPGGCIGGGGQPKPTNLEIRQARTQLTFKEDMDLPLRKSHDNPEIKAIYENYLKEPLGHNSHHYLHTTYSSQKVRDMNLYNANEAAGLDEILAKYPKEKEYLMPIIIEEHDKKGYISDPSIVKISEHLGMYPAQIESILSSYHYFPREHTIAILMSICVHCHNCMMKGQGRLLKTIQETYDIHETHGGVAKDGSFTLHTLNWLGYCVNDAPAMMIKRKGTNYVETFTGLLGDNIDQRLKSLKNLKKELPKWPKNNIREMKSQRNGNSYSCMNTQAPIAEATKKAVSMGPEKVIEEVFKSNLVGRGGAGFRTGKKWESAYKTPASDKYVVCNADEGLPSTYKDWCLLNNEAKRKEVFTGMGICAKTIGAKRCFMYLRYEYRNLVPALEQSIKDVQSTCPELADLKYEIRLGGGPYVAGEENAQFESIEGRAPLPRKDRPGNIFPTMEGLFHKPTVINNVETFFAIPHIIQQGSQSFGEGKMPKLLSVTGDVDEPILIETNLNNYSLNHLLQEISAKDIVAAEIGGCTEPIIFGSKFDTLFGFGRGTLNAVGSVVLFNSSCDLGKIYENKLKFMAEESCKQCVPCRDGSYIFHRAFKELRDTGKSSYNMRALAVASESAARSSICAHGKALESLFKSACDFMNKTKPIYQPHSTYHQ

SEQ ID NO: 19 - protein, 467 aa, Spironucleus barkhanus
MKVRQSPFKIDITNGPIDRNDAIQIDYQKCIGCQMCAKTCTDSQNFNIFKISAPKTKPFVNAYGSVAEGTERNALAGTDCTGCGACVRACPVEALMPAFNIRPVLEPISEKKKVTIAVIAPSTRVGLAEGMGMGVGVTAERQMVYELKQMGFDYVFDNMWGADAPTTEDAKEILKAKAAGKTAFTSCCPAWVKLVETTYPELLPNISSARSPHGIICSVIKKYFAKDIGKKADELYVVGVMPCTAKKNEAARKELTTDGSPDCDISITTRELMAYLKEKKVTFSAAREIELKDNVQAQYDAPFNTFSGSAYIYGKTAGVTEAVVRYVCAIKKVPFSVGMITKELIWENKLHSSSLTLLTFSAAGEDYRICVSYGGLAAHKAVELYKSGELKVDAVEVMVCPGGCVGGGGQPKQPKKDMILKRHEGLDKHDKEAPYSNCTENPTLNEFYERIGTDVHHVMHTTYSAYK

SEQ ID NO: 20 - protein, 468 aa, Trichomonas vaginalis
MLASSATAMKGFANSLRMKDYSSTGINFDMTKCINCQSCVRACTNIAGQNVLKSLTVNGKSVVQTVTGKPLAETNCISCGQCTLGCPKFTIFEADAINPVKEVLTKKNGRIAVCQIAPAIRINMAEALGVPAGTISLGKVVTALKRLGFDYVFDTNFAADMTIVEEATELVQRLSDKNAVLPMFTSCCPAWVNYVEKSDPSLIPYLSSCRSPMSMLSSVIKNVFPKKIGTTADKIYNVAIMPCTRKKDEIQRSQFTMKDGKQETGAVLTSRELAKMIKEAKINFKELPDTPCDNFYSEASGGGAIFCATGGVMEAAVRSAYKFLTKKELAPIDLQDVRGVASGVKLAEVDIAGTKVKVAVAHGIKNAMTLIKKIKSGEEQFKDVKFVEVMACPGGCVVGGGSPKAKTKKAVQARLNATYSIDKSSKHRTSQDNPQLLQLYKESFEGKFGGHVAHHLLHTHYKNRKVNP

SEQ ID NO: 21 - protein, 449 aa, Trichomonas vaginalis
MLASSSRAAANIRWVDTSHNAIAFDMHKCINCQACVRACKNVAGQSVLKSVKINEGKKKGVVQTVTGKLLAETNCIGCGQCTLVCPTQAIHEKDALKQMNNIFKNKGDRILVCQIAPAIRINMRRPWCSSRNSFHRQSRYSPQRLGFDYVFDTNFGADLTIVEEATELLQRLNDPKAVLPMFTSCCPAWVNYVEKSYPQWMPHLSTCRSPIGMLSAVIKNVFPKHIGVDPKRIFSVGIMPCTAKKDEAAREQLMTKSGLHETDLDITSRELAKMIKAAKINFKELPDTELDSPYAMATGGGAIFCATGGVMEAAVRSAYKFATGKELAPIEFVQVRGAEKGIKVGTVDINGREIKVAVAQGVKNAMSLIKKIEEGQDDVKGVVFCEVMACPGGCVGGGGSPRAKTKAAMNKRLDATYRIDRASKYRTPQDNTQLQDLYNATWVVSLVMD

SEQ ID NO: 22 - protein, 589 aa, Trichomonas vaginalis
ASTGINSTANILRNITVTVNGKPLEAKKGETVLELCDRNNIRIPRLCFHPNLPPKASCRVCLVECDGKWLSPACVTTVWDGLKIDTKSKNVRDSVENNLKELLDCHDETCSACIANHRCQFRDMNVAYSVKAETKEICSEEGIDESTNAIRLDTSKCVLCGRCIRACEEVAGTSAIIFGNRAKKMRIQPTFGVTLQETSCIKCGQCTLYCPVGAITEKSQVKEALDILANKGKKITVVQVAPAVRVALSEAFGYKEGTVTTGKMVSALKALGFDLVYDTNYGADLTICEEAGELVNRLRDPNAKFPMFTTCCPAWVNYVEQSAPDFIPNLSSCRSPQGMLSALIKNYLPKLLDVKQEDVLNFSIMPCTAKKDEVERPELRTKSGLKETDMVLTVRELVEMIKLSNIDFNNLPDTQFDNIFGFGSGAGQIFAATGGVMEAASRTAFEVYTGKKLTNVNIYPVRGMDGLRIAELDLDGTKLKVAVCHGIANTAKLLDRLREKDPELMDIKFIEIMACPGGCVCGGGTPQPKNRVSLDNRLAAIYNIDAKMECRKSHENPLIKGVYKEFLGKPNSHLAHELLHTHFKHHPKW

SEQ ID NO: 23 - protein, 582 aa, Trichomonas vaginalis
MKTIILNGNEVHTDKDITILELARENNVDIPTLCFLKDCGNFGKCGVCMVEVEGKGFRAACVAKVEDGMVINTESDEVKERIKKRVSMLLDKHEFKCGQCSRRENCEFLKLVIKTKAKASKPFLPEDKDALVDNRSKAIVIDRSKCVLCGRCVAACKQHTSTCSIQFIKKDGQRAVGTVDDVCLDDSTCLLCGQCVIACPVAALKEKSHIEKVQEALNDPKKHVIVAMAPSVRTAMGELFKMGYGKDVTGKLYTALRMLGFDKVFDINFGADMTIMEEATELLGRVKNNGPFPMFTSCCPAWVRLAQNYHPELLDNLSSAKSPQQIFGTASKTYYPSISGIAPEDVYTVTIMPCNDKKYEADIPFMETNSLRDIDASLTTRELAKMIKDAKIKFADLEDGEVDPAMGTYSGAGAIFGATGGVMEAAIRSAKDFAENKELENVDYTEVRGFKGIKEAEVEIAGNKLNVAVINGASNFFEFMKSGKMNEKQYHFIEVMACPGGCINGGGQPHVNALDRENVDYRKLRASVLYNQDKNVLSKRKSHDNPAIIKMYDSYFGKPGEGLAHKLLHVKYTKDKNVSKHE

SEQ ID NO: 24 - protein, 497 aa, Chlamydomonas reinhardtii
MSALVLKPCAAVSIRGSSCRARQVAPRAPLAASTVRVALATLEAPARRLGNVACAAAAPAAEAPLSHVQQALAELAKPKDDPTRKHVCVQVAPAVRVAIAETLGLAPGATTPKQLAEGLRRLGFDEVFDTLFGADLTIMEEGSELLHRLTEHLEAHPHSDEPLPMFTSCCPGWIAMLEKSYPDLIPYVSSCKSPQMMLAAMVKSYLAEKKGIAPKDMVMVSIMPCTRKQSEADRDWFCVDADPTLRQLDHVITTVELGNIFKERGINLAELPEGEWDNPMGVGSGAGVLFGTTGGVMEAALRTAYELFTGTPLPRLSLSEVRGMDGIKETNITMVPAPGSKFEELLKHRAAARAEAAAHGTPGPLAWDGGAGFTSEDGRGGITLRVAVANGLGNAKKLITKMQAGEAKYDFVEIMACPAGCVGGGGQPRSTDKAITQKRQAALYNLDEKSTLRRSHENPSIRELYDTYLGEPLGHKAHELLHTHYVAGGVEEKDEKK

SEQ ID NO: 25 - protein, 415 aa, Chlorella fusca
AGPTSECDCPPTPQAKLPHWQQALDELAKPKESRRLMIAQIASAVRVAIAETIGLAPGDVTIGQLVTGLRMLGFDYVFDTLFGADLTIMEEGTELLHRLQDHLEQHPNKEEPLPMFTSCCPGWVAMVEKSNPELIPYLSSCKSPQMMLGAVIKNYYAQQVGVQPSDICNVSVMPCVRKQGEADREWFNTTGAGLARDVDHVVTTAEVGKIFLERGIKLNELPESNFDNPIGEGTGGALLFGTTGGVMEAALRTVYEVVTQKPMGRVDFEEVRGLEGIKEAEITLKPGDDSPFKAFAGADGQGITLKIAVANGLGNAKKLIKSLSEGKAKYDFIEVMACPGGCIGGGGQPRSTDKQILQKRQQAMYNLDERSTIRRSHDNPFIQALYDKFLGAPNSHKAHDLLHTHYVAGGIPEEK

SEQ ID NO: 26 - protein, 505 aa, Chlamydomonas reinhardtii
MALGLLAELRAGQAVACARRTNAPAHPAAVVPCLPSRAGKFFNLSQKVPSSQSARGSTIRVAATATDAVPHWKLALEELDKPKDGGRKVLIAQVAPAVRVAIAESFGLAPGAVSPGKLATGLRALGFDQVFDTLFAADLTIMEEGTELLHRLKEHLEAHPHSDEPLPMFTSCCPGWVAMMEKSYPELIPFVSSCKSPQMMMGAMVKTYLSEKQGIPAKDIVMVSVMPCVRKQGEADREWFCVSEPGVRDVDHVITTAELGNIFKERGINLPELPDSDWDQPLGLGSGAGVLFGTTGGVMEAALRTAYEIVTKEPLPRLNLSEVRGLDGIKEASVTLVPAPGSKFAELVAERLAHKVEEAAAAEAAAAVEGAVKPPIAYDGGQGFSTDDGKGGLKLRVAVANGLGNAKKLIGKMVSGEAKYDFVEIMACPAGCVGGGGQPRSTDKQITQKRQAALYDLDERNTLRRSHENEAVNQLYKEFLGEPLSHRAHELLHTHYVPGGAEADA

SEQ ID NO: 27 - protein, 403 aa, Scenedesmus obliquus
PHWQQTLDELAKPKERKVMIAQIAPAVRGIAETMGLNPGDVTVGQMVTGLRMLGFDYVFDTLFGADLTIMEEGTELLHRLQDHLEQHPNKEEPLPMFTSCCPGWVAMVEKSNPELIPYLSSCKSPQMMLGAVIKNYFAAEAGAKPEDICNVSVMPCVRKSGEAEPRSGSTHHRAGRRDVDHVMTTAELGKIFVERGIKLNELQESPFDNPVGEGSGGGLLFGTTGGVMEAALRTVYEVVTAEALGPQRSSLTTSTAWTPAQRASPRPSPQAPTAPSRPLQAQTESGITLNIAVANGLGNAKKLIKQLAAGESKYDFTEVMACPGGCIGGGGQPQRNKQILQKRQAAMYDLDERAVIRRTENPLIGALYEKFLGEPNGHKAHELLHTHYVAGGVPDRRSEGEAW

SEQ ID NO: 28 - protein, 581 aa, Thermoanaerobacter tengcongensis strain MB4T
MDKVRVTIDGITVEVPSYYTVLEAAKEAGIDIPTLCYLKEINQIGACRICLVEIEGVRNLQTSCTYPVFDGMKVYTNTPKIREARRLNLELILSNHDRNCLTCVRSTNCELQALAKRLGVEEIRFEGENIKYPIDDASPAVVRDPNKCVLCRRCVAVCSEVQNVFAIGMVNRGFKTMVAPSFGRSLKDSPCISCGQCIMVCPVGAIYEKDHTKRVYEALADDKKYVVAQTAPAVRVALGEEFGMPVGTIVTGKMAAALRRMGFDAVFDTNFAADLTIMEEGSELLERIKHGGKLPMITSCSPGWIAFCEKYYPEFIDNLSTCKSPHMMMGALVKSYYAEKKGLDPKDIFVVSIMPCTAKKLEIEREEMIRNGMKDVDAVLTTRELARMIKEMGIDFVNLKDEEFDEPLGMSTGAGAIFGATGGVMEAALRTVAEIVEGRDIGKIDFEEVRGLEGVREATITIDGMDIKIAIANGTGNAKKLLDKVKAGEVEYHFIEVMGCPGGCIMGGGQPIHNPNEMEEVKKLRAKAIYEIDKNLPIRKSHENPAIKRLYEEFLGYPLSEKSHELLHTHYSRKELYPLVK

SEQ ID NO: 29 - protein, 636 aa, Neocallimastix frontalis
MSMLSSVLNKAVVNPKLTRSLATAAAEKMVNISINGRKFQVKPKTTVLEAAKANGYYIPTLCYHQELPVAGNCRLCLVYAKGSWKPLTACTTEVWEGMEIETDSPAVIETVRSSLSMMREEHPNDCMTCGSNGDCEFQDLIYRYQIDAKHPVRSLLKHKSKKTNHSITEPCYSPFDNTTFSVARDMNKCVKCGRCIRACHHFQNINILGFINRAGYERVGTPMDRPMNFTKCVECGQCSQVCPVGAITARTEVVDVLRHLDTKRKVVVCSTAPAIRVAPAEEFSTEADFDFTGKMVAGLRKLGFDYIFDTNFSADLTIMEEGTELIDRLNNGGKFPMFTSCCPGWINMVEKSYPELSDNLSSCKSPQQMIGAVIKSYFAKKLGLSTEDIIHVSIMPCTAKKGEARRPEFVQKGKDGKDYPDIDYVITTRELLTLLKLKKINPAELPDDKFDSPLGIGSSAGNLFGVTGGVMEAAIRTAQVITGVENPIPLGELKAIRGLDGIKAANVPLKTKDGKEVSVRAAVVSGGANIQKFLEKIKNKELEFDFIEMMMCPGGCINGGGQPKSADPEIVAKKMQRMYTMDDQAKLRLCHENPEIIDVYKNFLGEPNSHLAHELLHTHYNDRSKTIHDMGHHEKK

SEQ ID NO: 30 - protein, 555 aa, Piromyces sp. E2
CLVDVKGSWKPLTACTTEVWEGMEIETDTPAVRETVRSSLAMMREEHPNDCMTCESNGNCEFQDLIYRYQIDAQHPVRTLLRNKFKKTNHSITEPCYSPFDDSTFSISRDMNKCVKCGRCVRACHHFQNINILGFINRAGYERVGTPMDRPMNFTKCVECGQCSQVCPVGAITERNECIEVLRHLDTKRKIVVVSTAPAIRVALAEEFNAEPDFDFTGKMVAGLKKLGFDYIFDTNFSADLTIMEEGTELITRLNEGGKFPMFTSCCPGWINMVEKSYPEIRDNLSSCKSPQQMIGAVIKTYFAKKINAKPEDIIHVSVMPCTAKKGEAKRPEFKRDGVPDIDHVITTRELITLLKLKRINPSELKNEKFDSPLGIGSSAGNLFGVTGGVMEAAVRTAQIITGVENPIPLGELKAIRGLDGIKAASVPLKTKDGKDVNVRAAVVSGGANIQKFLEKLKKKELEFDFVEMMMCPGGCINGGGQPKSADPKVVAKKMERMYTMDDQASLRLSHENPEITQIYKEFLKEPNGHLSHELLHTHYNDRSKAIQDMSLHQK

SEQ ID NO: 31 - protein, 389 aa, Neocallimastix frontalis
TERNEVIEVLRQLDSKRKILVCSTAPAIRVALAEEFNADPDFNFTGKMVAGLRKLGFDYIFDTNFSADLTIMEEGTELINRLNNGGKFPMFTSCCPGWINMVEKSYPELRENLSTCKSPQQMIGALIKSYFAKKLGVSTEDIIHVSVMPCTAKKGEAKRPEFVQKGKDGKNYPDIDYVLTTRELLTLMKLKKVNPAELADDKLDSPLGISSSAGNLFGVTGGVMEAAVRTAQIITGVENPIPLGELKAVRGLEGIKAATVPLKTKEGKDINVRAAVVSGGANIQKFLEKIKNKEVEFDFVEMMMCPGGCINGGGQPKSADPKIVTKKMQRMYTMDEQATLRLSHENEEVKQIYKEFLIEPNGHLSHELLHTHYNDRSKAIQDMSLHEKK

SEQ ID NO: 32 - protein, 458 aa, Desulfovibrio desulfuricans
MNGQQNVIRIDSDICTGCGRCKDVCPVGAVEGVQGTPHSIREDVCVLCGQCVQQCSAFASFYEQHPACIAEKKRERGLFVSEAAPLFAAWHTGDAPRVAGRLAEGCHSMVQCAPAVRAAIGEEFGMPAGALTPGRLAAALRRLGFDRVYDTNFAADLTIMEEGSELLQRMEGAGPLPMFTSCCPAWVRYAEQQFPDLLEHLSSCKSPQQMAGAVFKSYGAQLDGVDPRQVFSVAVMPCTCKKAEAQRPGMEHDGVRDVDAVLTTGELAAMLRQAHIDFAALPDEPFDRPLGSYSGAGNIFGLTGGVMEAALRTAYELVTGEPVPCTELVYVRGGEGIRHATLTMDGRTFRVAVVAGLQHVRPLLEAVRAGTCDVNFVEVMCCPQGCISGGGQPKVLLPFQRDEVYAARKAALYRHDAELACRKSHENPQVQALYREFLGEPLSHVSHNLLHTVYGQTR

SEQ ID NO: 33 - protein, 554 aa, Desulfitobacterium hafniense
MMQLKHPFQSGFQQQSCKRHTKKVVVDMESKAGKGSNLSRRSFLKFAGGAGIAGASLSLTGCGQPLTPASAVGGEGWMPTQYNEPGGWPTNVRGRVPIDPENPALRRDDQKCILCGQCIEVCKTIQSVYGNYELPLKNEIPCINCGQCIHWCPSGAISEREDIDQVAKALADPKITVVVQTAPATRIGLGEEFGLPVGTNVQGKQVAALRKLGFDVIFDTNFAADLTIMEEGTELVKRITGELHHPLPQFTSCCPGWVKFVEYYYPELLPNLSSAKSPQQMAGALVKTYFAEKNHVEPQKIFSVAIMPCTAKKFECQRPEMISAQTYWQDEQVSPDVDVVLTTRELARMIKRAGIDLPSLPDEEYDQLMGVATGAGAIFGTTGGVMEAAVRSAYYLVTGEQPPAALWQLTPVRGMEGVKEAAVSIPGAGEIRIAVISGLDNARAIMEQVKAGNSPWTFIEVMACPGGCQYGGGQPRSSAPPSDGVRNTRAASLYKIDAQAKLRNSHDNPQIKQVYAEFLTSPLSEKAEELLHTHYISRAEEFDAKKPQSHEYEV

SEQ ID NO: 34 - protein, 578 aa, Eubacterium acidaminophilum
MVNITIDGRQVTVPANSTVLDAARDMGINIPTLCYLKDINKTGACRMCLVEVEGIRNLQTACTFPVRDGLVVKTNTKRVRDARRDNLQLILSNHHRDCLSCFRNGSCELQALCDDMGLSELDFEAPKELKPVDMLSHSIVRDPNKCILCGRCVAVCNKVQEVGILAFTNRGVETEVAPAFATSMADAPCIYCGQCVNVCPVAALREKTDIEKVWEVLEDETKHVVVQVAPAVRAALGEMFGNPIGTRVTGKMFTALKMLGFQKVFDTNFAADLTIMEEGTELLGRIKNGGTLPMITSCSPGWIRYVEHFYPELLDHVSSCKSPQQMMGAVLKSYYAEKNNIAPENMIVVSVMPCIAKKTESAKEEMKNVHGTRDVDIVLTTRELGKMIKEARIEFNDLQDSNPDEFFGDYTGAAVIFGATGGVMEAAIRTVADIVSGQELEDIEYTAVRGLEGIKEAAVKIGDLEVKVAVAHGTANAGKLMDLVRDGKADYHFIEIMGCSGGCVTGGGQPHVDSRTKEKVNVKLERAKALYTEDKLRDKRKSHHNESVKRLYEEYLGKPNGHKAHELLHTHYKKRELF

SEQ ID NO: 35 - protein, 619 aa, Rhodopseudomonas palustris
MCTPDQASLSARDPAEATITLSINGVACAGFANETILSCARRYDVYIPTLCELEDIDHTPGACRVCLVEILQAGKDTPQIVTACNTPVRDGMEVQTRSKKARDMQRLQVELLMADHLQDCATCIRHGSCELQDLAQFVGLQQNRFFDRERTEARPVDHSSPSMVRDMRRCVRCQRCVAICRYHQKIDALAIEGSGLERMVALRDADGYPNSVCVSCGQCVLVCPTGALGERDETDRALDYICDPNVVTVVQFAPAVRVAFGEEFGLPAGTNVEGQIIAACRKLGVDVVLDTNFAADVVIMEEGAELLARLKQGRRPTFTSCCPAWINFAEIHYPDVLPLLSSTKSPQQVLSTIAKSYLPAQLGVPAERIRVISIMPCIAKKDEAVRPQMVHDGQPETDLVLTTREFARLLRREGIDLKDLPSSQFDRPFLSAYSGAGAIFGTTGGVMEAAVRTIYALVNGRELERIELTQLRGFEGLREATVDLGAPVGEVKVAMVHGLGDTRKLVESVLSGEANYDFIEVMACPGGCVDGGGSLRSKKAYLPLALKRRETIYNVDRAAKVRQSHNNPQVQALYRELLQAPNSEIAHRLLHTHYASRKRELQHTVKEIWDDLTMSTILY

SEQ ID NO: 36 - protein, 644 aa, Clostridium thermocellum
MDSFLMKGYIKEANIDYSCSRGSMEDLPKWEFREIPKVPRAVMPSLSLEERKNNFNEVELGLSEEVARKEARRCLKCGCSARFTCDLRKEASNHGIVYEEPIHDRPYIPKVDDHPFIVRDHNKCISCGRCIAACAEIEGPGVLTFYMKNGRQLVGTKSGLPLRDTDCVSCGQCVTACPCAALDYRRERGKVVRAINDPKKTVVGFVAPAVRSLISNTFGVSYEEASPFMAGLLKKLGFDKVFDFTFAADLTIVEETTEFLSRIQNKGVMPQFTSCCPGWINFVEKRYPEIIPHLSTCKSPQMMMGATVKNHYAKLMGINKEDLFVVSIVPCLAKKYEAARPEFIHDGIRDVDAVLTTTEMLEMMELADIKPSEVVPQEFDEPYKQVSGAGILFGASGGVAEAALRMAVEKLTGKVLTDHLEFEEIRGFEGVKESTIDVNGTKVRVAVVSGLKNAEPIIEKILNGVDVGYDLIEVMACPGGCICGAGHPVPEKIDSLEKRQQVLVNIDKVSKYRKSQENPDILRLYNEFYGEPNSPLAHELLHTHYTPKHGDSTCSPERKKGTAAFDVQEFTICMCESCMEKGAENLYNDLSSKIRLFKMDPFVQIKRIRLKETHPGKGVYIALNGKQIEEPMLSGNIPDESESE

SEQ ID NO: 37 - protein, 572 aa, Clostridium perfringens; identical to SEQ ID NO: 10

SEQ ID NO: 38 - protein, 583 aa, Clostridium thermocellum
MHVLKLVHSTQYWRAEEMDNREYMLIDGIPVEINGEKNLLELIRKAGIKLPTFCYHSELSVYGACRMCMVENEWGGLDAACSTPPRAGMSIKTNTERLQKYRKMILELLLANHCRDCTTCNNNGKCKLQDLAMRYNISHIRFPNTASNPDVDDSSLCITRDRSKCILCGDCVRVCNEVQNVGAIDFAYRGSKMTISTVFDKPIFESNCVGCGQCALACPTGAIVVKDDTQKVWKEIYDKNTRVSVQIAPAVRVALGKELGLNDGENAIGKIVAALRRMGFDDIFDTSTGADLTVLEESAELLRRIREGKNDMPLFTSCCPAWVNYCEKFYPELLPHVSTCRSPMQMFASIIKEEYSTSSKRLVHVAVMPCTAKKFEAARKEFKVNGVPNVDYVLTTQELVRMIKESGIVFSELEPEAIDMPFGTYTGAGVIFGVSGGVTEAVLRRVVSDKSPTSFRSLAYTGVRGMNGVKEASVMYGDRKLKVAVVSGLKNAGDLIERIKAGEHYDLVEVMACPGGCINGGGQPFVQSEEREKRGKGLYSADKLCNIKSSEENPLMMTLYKGILKGRVHELLHVDYASKKEAK

SEQ ID NO: 39 - protein, 439 aa, Desulfovibrio desulfuricans
MAGCKAQHPPAAYLAGLEVPAAGSEVTMEGVRYKMNAPKDVDPATIRFVEVDHDKCMACGECEYHCPTGVMQEVTEDGYRGVVDPVACVNCGQCLANCPFGAIHEEVSFVGELYEKLKDPDTVVVSMPAPAVRYALGECFGLPTGTYVGGQMHAALRRLGFNLVWDTEWTADVTIMEEGTELLERVKHGNMPLPQFTSCCPGWIKFAETFYPDLEKHLSTCKSPIAMIGPLAKTYGAQEAGVPAKKMYTVSIMPCIAKKFEGMRPEMNASGYRDIDATITTRELAWMIKKAGIDFTSLPSEEPDPALGMSTGAATIFCTSGGVMEAALRLAYEALSGGTLADPDIKVVRTHEGINTAEVPVPNFGTVKVAVASGLDNAAKLCEEVRAGKSPYHFIEVMTCPGGCVNGGGQPLEPGMLQSSLFKSTITKINRRFTRRSVA

SEQ ID NO: 40 - protein, 379 aa, Desulfovibrio desulfuricans
MNLVEMEKIQYVDQSPDPRANPDELFFIQIDPEKCIGCDTCQEYCPTGAIFGDTGSAHSIPHEEICINCGQCLTHCPVGAIYEVQSWVRELSEKIKDPEIKVIAMPAPAVRYGLGECFGMPVGTVTTGKMLTALQMLGFDHVWDNEFTADVTIWEEGTEFVKRLTGQIDKPLPQFTSCCPGWHKYVESFYPELFPHLSSCKSPIGMMGALAKTYGPDVMKYDRSKVYTVSIMPCTAKKYEGMRADLWSSGYKDIDATIDTRELAYMIKKAGIDFAALPDGKRDTLMGDSTGGATIFGVSGGVMEAALRYAYEAVTGKKPSSWDFTMVRGLNGIKEGTVTIGDAKINVAVVHGAKRFAEVCEVIKTGKSPCISSSLCLPR

SEQ ID NO: 41 - protein, 421 aa, Desulfovibrio desulfuricans
MNLVEMEKIQYVDQSPDPRANPDELFFIQIDPEKCIGCDTCQEYCPTGAIFGDTGSAHSIPHEEICINCGQCLTHCPVGAIYEVQSWVRELSEKIKDPEIKVIAMPAPAVRYGLGECFGMPVGTVTTGKMLTALQMLGFDHVWDNEFTADVTIWEEGTEFVKRLTGQIDKPLPQFTSCCPGWHKYVESFYPELFPHLSSCKSPIGMMGALAKTYGPDVMKYDRSKVYTVSIMPCTAKKYEGMRADLWSSGYKDIDATIDTRELAYMIKKAGIDFAALPDGKRDTLMGDSTGGATIFGVSGGVMEAALRYAYEAVTGKKPSSWDFTMVRGLNGIKEGTVTIGDAKINVAVVHGAKRFAEVCEVIKTGKSPWHFIEFMACPGGCVCGGGQPVMPGVLEAMDRKVSRTFAGLKERLNRMSSSKA

SEQ ID NO: 42 - protein, 369 aa, Trichomonas vaginalis
CDGKWLSPACVTTVWDGLKIDTKSKNVRDSVENNLKELLDCHDETCSACIANHRCQFRDMNVAYSVKAETKEICSEEGIDESTNAIRLDTSKCVLCGRCIRACEEVAGTSAIIFGNRAKKMRIQPTFGVTLQETSCIKCGQCTLYCPVGAITEKSQVKEALDILANKGKKITVVQVAPAVRVALSEAFGYKEGTVTTGKMVSALKALGFDLVYDTNYGADLTICEEAGELVNRLRDPNAKFPMFTSCCPAWVNYVEQSAPDFIPNLSSCRSPQGMLSALIKNYLPKLLDVKQEDVLNFSIMPCTAKKDEVERPELRTKSGPKETDMVLTVRELVEMIKLSNIDFNNLPDTQFDNIFGFGSGAGQIFAAT

SEQ ID NO: 43 - protein, 369 aa, Trichomonas gallinae
CDGKWLSPACVTTVWDGLRIDTKSKVVRDSVENNLKELLDCHDETCSSCVANHRCQFRDMNVAYSVKADTKEICSEEGIDESTHAIRLDTSKCVLCGRCIRACEEVAGTSAIIFGNRAKHMRIQPTFGGTLQETACIKCGQCTLYCPVGAITEKSQVKEALDILANKGKKVTVVQVAPAVRVALSEAFGYKEGTVTTGKMVSALKALGFDLVYDTNYGADLTICEEAGELVNRLKDPKAVFPMFTSCCPAWVNYVEQSAPDFIPNLSSCRSPQGMLSSLIKNYLPKLLGIKQEEVMNFSIMPCTAKKDEIERPELQTKTGLKETDMVLTVRELVEMIKLSNIDFNNLPDTPFDNIFGFGSGAGQIFAAT

SEQ ID NO: 44 - protein, 456 aa, Nyctotherus ovalis
MISRLIAKKAPLFLRTFATSEMISLKIDGKIISVPKGIMLADAIKKAGANVPTMCYHPDLPTSGGICRVCLVESAKSPGYPIISCRTPVEEGMEIVTQGSKMKEYRQANLALMLSRHPNACLSCTSNTNCKTQELSANMNIGQCGFANATPPKNDDSYDMTTAIERDNDKCINCDICVHTCSLQGLNALGFYNEEGHAVKSMGTLDVSECIQCGQCINRCPTGAITEKSEIRPVLDAINIQQRLVFQMAPSIRVAVAEEFGIKPGEKILKNEIATALRKLGSNVFVLDTNFSADLTIIEEGHELIERLYRNVTGKKLLGGDHMPIDLPMLTSCCPGWIMFIEKNYPDLLNNLSTCKSPQGMLGALIKGYWAKNIKKMDPKDIVSVSIMPCTAKKAEKERPQLRGDEGYKDVDYILTTRELAKMLKQSNIDLAKMEPTPFDKVMSEGTGAAVIFGVT

SEQ ID NO: 45 - protein, 369 aa, Trichomonas vaginalis
CDGKWLAPACVTTVWDGLKIDTKSKMVKESVENNLKELLDCHDETCSSCVANHRCQFRDMNVAYSIKAETKEECSEEGIDESTNSIRLDTSKCVLCGRCIRACEEVAGQSAIIFGNRAKHMRIQPTFGQTLQDTSCIKCGQCTLYCPVGAITEKSQVKQALDILSNKGKKISVIQVAPAVRVALSEAFGYKEGSVTTGKMVSALKALGFDYVYDTNYSADLTIVEEAGELVQRLKNPNAVFPMFTSCCPAWVNYVEQSAPDFIPNLSSCRSPQGMLSSLVKNYLPKVLNIPVEDVLNFSIMPCTAKKDEIERPELRTKDGHKETDMVLTVRELVEMIKLSGIDFNNLPDTPFDSIFGFGSGAGQIFAAT

SEQ ID NO: 46 - protein, 464 aa, Entamoeba histolytica
RLHTVTGHDHNHSIQFDWSKCMGCGMCATKCTFGVLVKQPPKIPPFVQPNREKLSQENTDKTRVLIDESECTGCGQCSLVCNFGSITPIDHLVDTFKAKEAGKKLVAMIAPSTRLGVAEAMGMPIGSTAMAQLVHCLRLIGFDYVFDVDAGADKTTMDDYAEVIEMKKEGKGPAITSCCPAWIELVEKEYPDLIPNVSTARSPIGCLAGCIKRGWAKDVGIAVEDLYTVGIMPCIAKKTESQRQQIHQDYDASCTSNEIAAYFKKHLPPEECKFTQEREEALAKTEDGQCDLPFRRISGGSNIFGRTGGVCETVLRVIARNAGVDWNSCTVNKEETFKHAASGSTMTNLSVDIGGTIITGAVCHGGYAIRHACELIRKGELKVDVVEMMACVGGCLGGAGQPKIPPAKKLEMDKRRVMLDILDQQTDIRAANENTDVLGWIDKHFDHQGAHQHLHTYFTPRYQN

SEQ ID NO: 47 - protein, 474 aa, Giardia intestinalis
MPPKPQHDVTGVDSNNAIMIDYAKCIGCNMCIKACDVQGIGVYKQNEKPKYPPIVKLSTLFNSDCIGCGQCATICPVDAIAPKNNLEIYKGESASKKVRVALIAPSTRVAFGDVFGLPIGTNTIYSLIRMLKQYLGFDYVFDVNFGADETTVIDTQELLHFKHEGRGPVFTSCCPAWVNLCEMKYPELLPQVSTAKSCVAMVATLVKRRWVQEHLIPKGIVDSVDDVYVADIMPCTAKKDESMRPQLNRDVDICLTVREVAEHLYFLHGARLTLEEVEADALVLRPGRSTQKKWDFDAPFNTVSGGSHIFGKTGGVAETCLRFISYMKKSPIENVKEELLKEFKTPGQLVQTVKLVSCEIAGETYRALIAHGGSAINAAARMVLNKEVECDVVEQMACPGGCQNGGGMPKIKGKKEAVLTRASTLDILDGKERFASAGENKTLWGFNGCLTEHEAHELLHTHYQHRPVESLLPQ

SEQ ID NO: 48 - protein, 844 aa, Desulfitobacterium hafniense
MVKIISITNNAKRQGKGTSRKEKQAMKEVTKQQRIRVTVNGRQMEVYGDLTILQALLQEDIHIPHLCYDIRLERSNGNCGLCVVELGEGSEQQDVKACHTPIQEGMIIHTNSPRLEHYRKIRLEQILADHNADCVAPCVMTCPANIDIQSYLSHAGNGNFETAIKVIKERNPFPIVCGRVCPHSCEAQCRRNLIDEPVAINHVKRFIADWDIAHEQPWAPRKKAATGKKIAVVGAGSSGLSAAYYSAIQGHDVTVFERHPRAGGMMRYGIPEYRLPKETLDREIGLIADLGVKIMTNKALGTHIRLEDLHQDFDAVYLAIGSWRATPLQIEGDNLEGVWLGINFLEQVTKGADIKLGEHVVVIGGGNTAIDCARTALRKGAGSVKLVYRRTREEMPAESYEVEEAIHEGVEMYFLTAPHKIVAEGGRKLLHCIKMTLGEPDRSGRRRPIPIEGSETAFEADTIIGAIGQSTNTQFLYHDLPVKLNKWGDIEINGKTMQTSEMNIFAGGDCVTGPATVIQAVAAGRHAAEAMDSFLMKGYVKEQPMDYSCSRGSLEDLPQWEFEKIPRLKRAPMPALPPAERRDNFREVETGLSEETARAEARRCLKCGCYERYDCDLRQEASLHHVEFKKPVHERPYIPIVEDHSIIIRDHNKCISCGRCIAACAEVEGPDILSFYMKHGRQLVGTKSGLPLDQTDCVSCGQCVNACPCGALDYRSEIGRVFRAINDPGKTTVAFVAPAVRSVVSSQYGVSYQEASRFIAGLLKKIGFDKVFDFTFAADLTIVEETTEFLTRLQSHKPIPQFTSCCPGWVNFVERRYPEIIPYLSSCKSPQMMMGATVKITLRN

SEQ ID NO: 49 - protein, 119 aa, Nyctotherus velox
ILFMEKNYPDMLNHLSTCKSPQGMLGALIKGYWAKNVKKIDPKDVVSVSIMPCTAKKEEKDRITLKSDEGYNNVDYVLTTRELAKMFKQSNIDPSKLPPTQFDNVMSEGTGAAVIFGVT

SEQ ID NO: 50 - protein, 476 aa, Oryza sativa
MASSSSSASSRFSPALQASDLNDFIAPSQDCIISLNKGPSARRLPIKQKEIAVSTNPPEEAVKISLKDCLACSGCITSAETVMLEKQSLGDFITRINSDKAVIVSVSPQSRASLAAFFGLSQSQVFRKLTALFKSMGVKAVYDTSSSRDLSLIEACSEFVTRYHQNQLSSGKEAGKNLPMLSSACPGWICYAEKTLGSFILPYISAVKSPQQAIGAAIKHHMVGKLGLKPHDVYHVTVMPCYDKKLEAVRDDFVFSVEDKDVTEVDSVLTTGEVLDLIQSRSVDFKTLEESPMDRLLTNVDDDGQLYGVSGGSGGYAETVFRHAAHVLFDRKIEGSVDFRILRNSDFREVTLEVEGKPVLKFALCYGFRNLQNIIRKIKMGKCEYHFIEVMACPSGCLNGGGQIKPAKGQSAKDLIQLLEDVYIQDVSVSNPFENPIAKRLYDEWLGQPGSENAKKYLHTKYHPVVKSVASQLQNW

SEQ ID NO: 51 - protein, 114 aa, Psalteriomonas lanterna
INLVEKHYPEYLPNLSSCRSPQGMLSSLIKNYWAKKMGIEPKDVVVVSFMPCGAKKDEIKRPQLKGETDYVLTTRELGKLFKMGGLNDLSVLEPVKYDDPLGESTGAAVIFGAT

SEQ ID NO: 52 - protein, 119 aa, Nyctotherus ovalis
IMFMEKNYPDMLNHLSTCKSPQGMLGALIKGYWAKNIKKMDPKDIVSVSIMPCTAKKAEKERPQLRGDEGYKDVDYILTTRELAKMLKQSNIDLGKMEPTPFDKVMSEGTGAAVIFGVT

SEQ ID NO: 53 - protein, 119 aa, Nyctotherus ovalis
IMFMEKNYPDMLNHLSTCKSPQGMLGALIKGYWAKNVKKMDPKDIVSVSIMPCTAKKAEKERPQLRGDEGYKDVDYILTTRELAKMLKQSNIDLGKMEPRPFDKVMSEGTGAAVIFGVT

SEQ ID NO: 54 - protein, 520 aa, Rhodospirillum rubrum
MRPVQRPRRWPGLRQRLSPERPVDRRSRRRSGAARPGRRRGSGVQHEILRSVSQRDMSMSIQPTVTIDPELCTGCGRCVETCPVQAIAGSRGKAHEIEAAACVSCGRCVATCAAFDSIFDAFPTPRPVRLKRRGLPGSLKEPLFAAHDPSRIEAVRKAFATPKRMTVMQVDTMACVALAEDFGLPPGSLSPLKIASAARQLGFDRVYRTSFPAGLAVLETAHEMAARLANGGNLPVINSSCPAVVAFLERRYPELLHYLSTVKSPHQIAGALYNSYLADAANLAPANIHKVSVVACLSHKAEAERPEMMTCGCPDIDTVLTARELAILIKDAGIDVPLLGDGEFDNDFPEIEGLDTLYCAPGDVSRAVLGAGRWFLGQGEGVGAPAGETVEVLDEATRLTRLAYPGGTLQALTVAGFDKAVPYLEAIKAGRNAFQFLEIASCPQGCASGAGLPKVLLETEKPARYRARIENLPPAAPEAWSRLPGHPSIVALYGGYFGKAIGDKSNRRLHTQYAEPAAAP

SEQ ID NO: 55 - protein, 240 aa, Desulfitobacterium hafniense
MAVEKLTGEVLTDQLDYQEVRGLQGIKEAAVEAKGKKVNVAVISGLHNVEPILEKIIEGMEVGYDLIEVMACPGGCICGAGHPVPEKIDTLEKRQQVLVNIDQTSRYRKSQENPDILRLYDEYYGEANSPLAHKLLHTHYEAVKREPVAKHDRRMADSAFVTHELTLCTCDKCTAQGSRELFAALSGKIRKLKMDSFVTARTIRLKENHPGQGVYAAIDGKLIETPVEQLEQRIFQHLIR

SEQ ID NO: 56 - protein, 86 aa, Desulfitobacterium hafniense
MVSIVPCIAKKYEAARPEFRSEGIRDVDAVLTSTEMLEMADIKLIEPADVEPQDFCEPYKRVSGAGILFGASGGVAKRPCGWRWRN

SEQ ID NO: 57 - protein, 477 aa, Drosophila melanogaster
MSRLSRALQLTDIDDFITPSQICIKPVQIDKARSKTGAKIKIKGDGCFEESESGNLKLNKVDISLQDCLACSGCITSAEEVLITQQSREELLKVLQENSKNKASEDWDNVRTIVFTLATQPILSLAYRYQIGVEDAARHLNGYFRSLGADYVLSTKVADDIALLECRQEFVDRYRENENLTMLSSSCPGWVCYAEKTHGNFLLPYVSTTRSPQQIMGVLVKQILADKMNVPASRIYHVTVMPCYDKKLEASREDFFSKANNSRDVDCVITSVEVEQLLSEAQQPLSQYDLLDLDWPWSNVRPEFMVWAHEKTLSGGYAEHIFKYAAKHIFNEDLKTELEFKQLKNRDFREIILKQNGKTVLKFAIANGFRNIQNLVQKLKREKVSNYHFVEVMACPSGCINGGAQIRPTTGQHVRELTRKLEELYQNLPRSEPENSLTKHIYNDFLDGFQSDKSYDVLHTRYHDVVSELSISLNINW

SEQ ID NO: 58 - protein, 538 aa, S. pombe
MAKLSVNDLNDFLSPGAVCIKPAQVKKQESKNDIRIDGDAYYEVTKDTGETSELGIASISLNDCLACSGCITSAETVLVNLQSYQEVLKHLESRKSQEILYVSLSPQVRANLAAYYGLSLQEIQAVLEMVFIGKLGFHAILDTNASREIVLQQCAQEFCNSWLQSRAHKNQNQVTNSVVNEHPLIPHSTSQISGVHSNTSSNSGINENAVLPILSSSCPGWICYVEKTHSNLIPNLSRVRSPQQACGRILKDWAVQQFSMQRNDVWHLSLMPCFDKKLEASRDEFSENGVRDVDSVLTPKELVEMFKFLRIDPIELTKNPIPFQQSTDAIPFWYPRITYEEQIGSSSGGYMGYVLSYAAKMLFGIDDVGPYVSMNNKNGDLTEYTLRHPETNEQLISMATCYGFRNIQNLVRRVHGNSSVRKGRVLLKKRVRSNAQNPTEEPSRYDYVEVMACPGGCINGGGQLPFPSVERIVSARDWMQQVEKLYYEPGTRSVDQSAVSYMLEQWVKDPTLTPKFLHTSYRAVQTDNDNPLLLANKW

SEQ ID NO: 59 - protein, 119 aa, Metopus contortus
IIFAEKNYPEMVNHLSTTKSPMQMLSSLSKGYWAKEGKKIDPKNVVNVAIMPCTAKKAWKERPDMKADNGDPVTDYVLTTRELGTMLRQSNINPVSLPKTPFDKIMGESTGAAVIFGAT

SEQ ID NO: 60 - protein, 462 aa, Mus musculus
MKCEHCTRKECSKKSKNDDQENVSSDGAQPSDGASPAKESEEKGEFHKLADAKIFLSDCLACDSCVTVEEGVQLSQQSAKDFLHVLNLNKRCDTSKHRVLVVSVCPQSLPYFAAKFNLSVTDASRRLCGFLKSLGVHYVFDTTIAADFSILESQKEFVRRYHQHSEEQRELPMLTSACPGWVRYAERVLGRPIIPYLCTAKSPQQVMGSLVKDYFARQQNLSPEKIFHVVVAPCYDKKLEALREGLSTTLNGARGTDCVLTSGEIAQIMEQSDLSVKDIAVDTLFGDMKEVAVQRHDGVSSDGHLAHVFRHAAKELFGEHVEEITYRALRNKDFHEVTLEKNGEVLLRFAAAYGFRNIQNMIQKLKKGKLPYHFVEVLACPRGCLNGRGQAQTEDGHTDRALLQQMEGIYSGIPVRPPESSTHVQELYQEWLEGTESPKVQEVLHTSYQSLEPCTDGLDIKW

SEQ ID NO: 61 - protein, 457 aa, Caenorhabditis elegans
MEDSGFSGVVRLSNVSDFIAPNLDCIIPLETRTVEKKKEESQVNIRTKKPKDKESSKTEEKKSVKISLADCLACSGCITSAETVLVEEQSFGRVYEGIQNSKLSVVTVSPQAITSIAVKIGKSTNEVAKIIASFFRRLGVKYVIDSSFARKFAHSLIYEELSTTPSTSRPLLSSACPGFVCYAEKSHGELLIPKISKIRSPQAISGAIIKGFLAKREGLSPCDVFHAAVMPCFDKKLEASREQFKVDGTDVRETDCVISTAELLEEIIKLENDEAGDVENRSEEEQWLSALSKGSVIGDDGGASGGYADRIVRDFVLENGGGIVKTSKLNKNMFSTTVESEAGEILLRVAKVYGFRNVQNLVRKMKTKKEKTDYVEIMACPGGCANGGGQIRYETMDEREEKLIKVEALYEDLPRQDDEETWIKVREEWEKLDKNYRNLLFTDYRPVETNVAQVLKW

SEQ ID NO: 62 - protein, 462 aa, Mus musculus
MKCEHCTRKECSKKSKTDDQENVSSDGAQPSDGASPAKESEEKGEFHKLADAKIFLSDCLACDSCVTVEEGVQLSQQSAKDFLHVLNLNKRCDTSKHRVLVVSVCPQSLPYFAAKFNLSVTDASRRLCGFLKSLGVHYVFDTTIAADFSILESQKEFVRRYHQHSEEQRELPMLTSACPGWVRYAERVLGRPIIPYLCTAKSPQQVMGSLVKDYFARQQNLSPEKIFHVVVAPCYDKKLEALREGLSTTLNGARGTDCVLTSGEIAQIMEQSDLSVKDIAVDTLFGDMKEVAVQRHDGVSSDGHLAHVFRHAAKELFGEHVEEITYRALRNKDFHEVTLEKNGEVLLRFAAAYGFRNIQNMIQKLKKGKLPYHFVEVLACPRGCLNGRGQAQTEDGHTDRALLQQMEGIYSGIPVRPPESSTHVQELYQEWLEGTESPKVQEVLHTSYQSLEPCTDGLDIKW

SEQ ID NO: 63 - protein, 119 aa, Neocallimastix
IMFAEKNFPDMVNNLSTTKSPMQMLSSLTKGYWAKDIKKINPKDVVNVAIMPCTAKKQEKDRPGMKTDEGDKVTDFVLTTRELGMMLRQANIDPTKLPGTKFDKVMGESTGAAVIFGAT

SEQ ID NO: 64 - protein, 119 aa, Nyctotherus ovalis
IIFMEKNYPDMLSHLSTCKSPQGMLGALIKGYWAKKVKKVDPKDVVSVSIMPCTAKKAEKERPQLRGDEGFKDVDYVLTTRELAKMLKQSNIDLGKVEPTPFDAVMSEGTGAAVIFGVT

SEQ ID NO: 65 - protein, 490 aa, Clostridium perfringens
MAIKDANKQYIKFDTAVQVLKYEVLKRIAEKEFDGTLDKEKLNIAKEIVDDLKPNVRCCIYKERAIVEERMKLALGGHENRENMIEVIDIACDECPVNRFIVTDACRGCLAKKCRDSCNFGAISFDNRKCKIDYEKCKECGKCKEVCPYNAIAEVKRPCMRACIPKALSYDVDSKKAVIDDSKCIQCGACVVDCPFGAIMDKSYLVDVIRLLKDEKKVYAIVAPAISSQFNHSKIGKVITAIKKLGFEDVFEAALGADLVAVHECNEFKEKGELDFMTTSCCPAFVSYIEKNYPELKECISNTVSPMVAMARLIKSQNKDVKTVFIGPCIAKKTEAKRNEVSGDVDYVLTFEELLALLDSRNIKIDECEESDTKHGSFYGRLFARSGGVTESVKHLIDSEGIKVDFRPILGDGIKDCDIKLRLAKLKRAQGNFLEGMACKGGCINGPGSLNHDIKNSKEVDKYGELSSSEKIKDTLADIKFEDLNLSKNE

SEQ ID NO: 66 - protein, 456 aa, Homo sapiens
MKCEHCTRKECSKKTKTDDQENVSADAPSPAQENGEKGEFHKLADAKIFLSDCLACDSCMTAEEGVQLSQQNAKDFFRVLNLNKKCDTSKHKVLVVSVCPQSLPYFAAKFNLSVTDASRRLCGFLKSLGVHYVFDTTIAADFSILESQKEFVRRYRQHSEEERTLPMLTSACPGWVRYAERVLGRPITAHLCTAKSPQQVMGSLVKDYFARQQNLSPEKIFHVIVAPCYDKKLEALQESLPPALHGSRGADCVLTSGEIAQIMEQGDLSVRDAAVDTLFGDLKEDKVTRHDGASSDGHLAHIFRHAAKELFNEDVEEVTYRALRNKDFQEVTLEKNGEVVLRFAAAYGFRNIQNMILKLKKGKFPFHFVEVLACAGGCLNGRGQAQTPDGHADKALLRQMEGIYADIPVRRPESSAHVQELYQEWLEGINSPKAREVLHTTYQSQERGTHSLDIKW

SEQ ID NO: 67 - protein, 408 aa, Homo sapiens
MKCEHCTRKECSKKTKTDDQENVSADAPSPAQENGEKCDTSKHKVLVVSVCPQSLPYFAAKFNLSVTDASRRLCGFLKSLGVHYVFDTTIAADFSILESQKEFVRRYRQHSEEERTLPMLTSACPGWVRYAERVLGRPITAHLCTAKSPQQVMGSLVKDYFARQQNLSPEKIFHVIVAPCYDKKLEALQESLPPALHGSRGADCVLTSGEIAQIMEQGDLSVRDAAVDTLFGDLKEDKVTRHDGASSDGHLAHIFRHAAKELFNEDVEEVTYRALRNKDFQEVTLEKNGEVVLRFAAAYGFRNIQNMILKLKKGKFPFHFVEVLACAGGCLNGRGQAQTPDGHADKALLRQMEGIYADIPVRRPESSAHVQELYQEWLEGINSPKAREVLHTTYQSQERGTHSLDIKW

SEQ ID NO: 68 - protein, 502 aa, Homo sapiens
MKCEHCTRKECSKKTKTDDQENVSADAPSPAQENGEKGEFHKLADAKIFLSDCLACDSCMTAEEGVQLSQQNAKDFFRVLNLNKKCDTSKHKVLVVSVCPQSLPYFAAKFNLSVTDASRRLCGFLKSLGVHYVFDTTIAADFSILESQKEFVRRYRQHSEEERTLPMLTSACPGWVRYAERVLGRPITAHLCTAKSPQQVMGSLVKDYFARQQNLSPEKIFHVIVAPCYDKKLEALQESLPPALHGSRGADCVLTSEISQAWWCTPVITATREAAARESLEPGRQRLQRDKIAPLDSSLGGGGEIAQIMEQGDLSVRDAAVDTLFGDLKEDKVTRHDGASSDGHLAHIFRHAAKELFNEDVEEVTYRALRNKDFQEVTLEKNGEVVLRFAAAYGFRNIQNMILKLKKGKFPFHFVEVLACAGGCLNGRGQAQTPDGHADKALLRQMEGIYADIPVRRPESSAHVQELYQEWLEGINSPKAREVLHTTYQSQERGTHSLDIKW

SEQ ID NO: 69 - protein, 448 aa, Clostridium tetani
MHNDYREIFKRLSKSYYDDTFEKEVENILSSHSMDREKLAKIISILCGVNIEHSENYISNLKNAIKNYTASAEKVVTKLPCSTQCAKDGDIICEKSCPVNAIFRDPNDNNIYINDELCLDCGLCVRNCPSGSILDKKEFIPLAELLKSESIVIAAVAPAIMGQFGENTTINQLRTAFKKLGFTDMVEVAFFADMLTLKEAVEYDHFVKDEQDFMITSCCCPMWVGMLKKVYNDLVKYVSPSVSPMIAAGRVLKLLNPNCKVVFVGPCIAKKAEAREKDLLGDIDFVLTFTELRDIFDVFDIQPENLEEDFSSEYASKGGRLYARTGGVSIAVSEAIEKLFPNKYKFLKTIQADGVKGCKSLLDKIKQEDISANFVEGMGCVGGCVGGPKVIIDPSEGRNAVNNFAENSSIKVSVDSNCMNDILSKININSVEDFKDKDKISIFEREFK

SEQ ID NO: 70 - protein, 459 aa, Desulfovibrio desulfuricans
MYFRTYDNTINFEIMVRIAKAFHGDSFEEQVARIPLEMRPRKAHSSRCCIYRDRAIIRYRCMAMLGYAIEDETDELTSLSQYAKGALERDSIQGSMLTFIDEACNGCVRTHYEATSACRGCLAEACVQHCPKDAVRIVDGKSRIDPDKCVQCGKCMNVCPYHAIVQIPIPCEESCPTGAISKDECGKQVIDYDRCIFCGKCMAACPFAAVLEKSQMIDVLRRIREGRKVVAIVAPAIAGQVQAPMSRLATALRQLGFADVAEVASGADTTARLEADEFVERMEHGAAFMTSSCCPAYTQLVDKHLPELAPFVSDTRTPMHYTAAMVKDHDPDMVTVFIGPCVAKRNEGKHDELVDHVLTFQEMVAMLTAAGISVDACEDGRFMFPAMREGRSFPVSGGVTAGVQAHIGTRAEVRPLSVDGLNKKTFRQLKTWAKKGCEGNFVEVMGCQGGCVAGPAIVM

SEQ ID NO: 71 - protein, 494 aa, Clostridium tetani
MIVFENQLKKLKYLVLKEVAKMTLEDRLGEEDIERISFDIIKGDKAEYRCCVYKERAIVYERAKLATGCLPNGQVAEEFVHVEDDDQIIYVIDAACDKCPINKYVVTEACRGCLQHKCMEVCPAGSINRAAGKAYINHETCKECGLCESACPYNAIAEVMRPCRRACPTGALQMNLEDNKATINKEDCINCGSCMSVCPFGAISDKSYIVDITKALKNNKKVYAMVAPAITGQFGKDVSVGKMKNAFKAMGFEDMLEVACGADAVAAHESEEFIERLESGKKYMTTSCCPGFLGYIEKKFPDQLENVSNTVSPMVAIGRMIKKEYEDSVVVFVGPCTAKKAEIKRKGIKDAVDYVMTFEEIAALMGAFEIDPAECEEEDINDGSNYGRGFAQGGGVVSAIQNCIKDKEGIKFNPLRVSGPDQIKRAMIMAKVGKLSENFIEGMMCEGGCIGGPATMVSAVKAKAPLMKFSKSSTIKDVKDNEVLDKYKDINMER

SEQ ID NO: 72 - protein, 203 aa, Arabidopsis thaliana
MDLIKLKGVDFKDLEESPLDRVLTNVTEEGDLYGVAGSSGGYAETIFRHAAKALFGQTIEGPLEFKTLRNSDFREVTLQLEGKTVLKFALCYGFQNLQNIVRRVKTRKCDYQYVEIMACPAGCLNGGGQIKPKTGQSQKELIHSLEATYMNDTTLNTDPYQNPTAKRLFEEWLKEPGSNEAKKYLHTQYHPVVKSVTSQLNNW

SEQ ID NO: 73 - protein, 449 aa, Clostridium perfringens
MNKKYNSLFKELISSYYSEDNFDEKLNDIVKNNFNSKEDAIEVLSSLCGVDIDKNSDNIAYDIRKAITTHKIKKNIVDKVSVCTKNCSKESKGKCQSLCPFDAILTDPIDNSKYIDPNLCQNCGICVQVCESGHFLDRIELLPIIDLIKNNETVIAAVAPAIAGQFGENVSLDMLREAFIKIGFSDMIEVAFAADMLSIKEAVEFNHHVEKTGDILITSCCCPMWVAMLRKCYKDLVKDVSPSVSPMIAAGRVIKKLNKDAKVVFIGPCIAKKAEAREKDLVGAIDYVLTFEELNGIFEALKIDPSSMKGVPSIEYTSRGGRLYARTGGVSEAINDVVKELYPDKAKIFKAVQANGVKECKELLNKVQSGELKANFIEGMGCVGGCVGGPKRIVDPSIGKKHVDEVAYNSPIKVATHSHTMDEVLLRLGINSLKSFEDKEKISIFEREF

SEQ ID NO: 74 - protein, 359 aa, Desulfitobacterium hafniense
MAQSEIMKIRRQVLKSALDWVSHDQNRKDRATLARQIIPDGTPRYRCCIHKERAVIEERLKAVLEPDEGPIVRVLKEGCNGCEMHRYSVTDHCQNCVGHFCFTNCPKKAILFINNKAFIDQTRCVECGLCARNCPYHAIIEYRRPCEDSCPTKAISVREDRIASIAEAHCTSCGKCIISCPFGAVAESSQLIHLFEAVRNPEHKIYAVIAPAFVGQFGRKVSPGQVKSALLKLGFQDVLEAALGADRTIELEAREYDERLAHGEEFMTSSCCPAYVSAVIKEKPDLFHHISSTLSPMAQVAHILKEKDPEAKIAFIGPCVAKKEEGKRPETKVDFVLTFEELMVWLDYAGINPAEESEQ

SEQ ID NO: 75 - protein, 790 aa, Geobacter metallireducens
MCHWLHREAGLVYDPAVDQAINRVSGLTLSAGRTMEPIITVKEKCRKCYCCVRSCPVKAIKVAKSYTEIIVDRCIGCGNCLSNCPQQAKMVADKVGVTEKLLSSGEEVIAVLGSSFPAFFHNVTPGQLVAGLRKIGFAEVHEGSYGAELIADDYARITSEKGHPRISSHCPAIVDLIERHYPKLVGNLVPVVSPMVAMGRYLKGTLGQHVRVVYISSCVANKLETQTQETRGAVDIVLTYRELEGIFRSRQIALPALADEPLDGIRPGAGRLFPIADGTFRAFGIPFDPLDTEIVAACGEVNVMGIINDLAAGRISPRIADLRFCYDGCIGGPGRNRALTEFYRRNRVIAHFKQEVPCRTVPNSLLEAGRVSFGRSFASKYAKLEAPKANDVRKILNATNKFTVKDELNCRACGYRTCREYAVAVFQGLAEIEMCLPYNLQQLEEDRGRLIQKYELARRELEREYGDEFIVGNDRKTLDVLGLIKQVGPTPTTVLIRGESGTGKELTARAIHRYSKRNDKPLVTVNCTTITDSLLESELFGHKRGAFTGAVADKKGLFEAADGGTIFLDEIGDITPKLQAELLRVLDMGEVRPVGGTAAKKVDVRLIAATNKNLEQGVREGWFREDLYYRLNVFTITMPPLRSRVESIPILVHHFMDKASTKLNKRMVGIEDRAVKALTKYPWPGNIREMQNVIERAAVLTHDGVIRVENFPLALSEGLEEGFATGLDIHAASFRSEREQHMGKLEKKLIQRYLTEANGNISRAAKLANIPRRTFYRLLDKYRLRERDVR

SEQ ID NO: 76 - protein, 450 aa, Clostridium acetobutylicum
MNNKYIELFKSLVDSYYNDTFDSFVYHILSDEEVDKKELSKVISSLCGVSVEFKDTETYISELKKAISNYKCTDNIVEKIKECDSSCHSNEGETPCQKSCPFDAILVDKNTKTSHIQKDLCTDCGNCITSCPSGSILDKIEFMPLLNLFKNNETVIAAVAPAIAGQFGENVSLEMLRTAFKKVGFADMVEVAFFADMLTIKEAFEFNELVNSKDDLMITSCCCPMWVSMIRKIYKDLARHVSPSVSPMIASGRVIKKLNPNCKVVFIGPCIAKKAESRSQDISDAIDFVLTFEELKGIFDVLDIDPEKLPETHTKSYASREGRLYGRTGGVSTSVDEAVKRIFPNKHHLFKSTKVDGVKDCKDILNKTQAGNIGANFLEGMGCVGGCVGGPKAIVHKDQGRESVNKTAESSEIKISVDSERMKDILSRIGINSIEDFGDKSKVDIFERRF

SEQ ID NO: 77 - protein, 106 aa, Shewanella oneidensis
MNKKKHLFAEDSFFLSRRKFMAVGAAFVAALAIPIGWFTSKLERRNEYIKARSQGLYKDDSLAKTRVSHANPAVEKYYKEFGGEPLGHMSHELLHTHFVDRTKLSS

SEQ ID NO: 78 - protein, 504 aa, Entamoeba histolytica
MSTQLTPLRNKIISEVVKCFKSGRFIEDIDKLPTILTDGDGWKPTSKFVHSREQEEGIYREKVLSVLGFVDGEYDDITPLHVYAQKALERTSLHEPVFGISQKGCNKCHFNGYFVTQACEGCTSRPCSVNCPKKCISFGEDGRAVINQNNCIKCGRCYKFCPYGAIISKSVPCVKACPCGAMLDSPEGVKTIDFEKCINCGGCMRACPFGAILPRSNLIDVLKILPTKKVVACPAPSIAAHFGKYDLALVSGGLIQVGFTSVEDVSYGADLCALNEAKEFEERIVKNKKDFMTTSCCPAYINAINKHMPELKENVSHTPTPMHFATQAVKDRDQETVTVFIGPCNAKRWETLQDSTTDYCLTFDEIFGLFEGSGIDLSKVQPYTFVDKAHKEGKIFAVSGGVASAVASLLPKEVPDGVIKPTIIDGFSQENFKRLKNFKKNITGNLVEVMVCEGGCAYGPGCPGLNTPATSAKIKIAVDKMEAHPEGRWVGLPNSQIKPIKVEN

SEQ ID NO: 79 - protein, 560 aa, Cryptosporidium parvum
MFSTAVKLANLDDYLESSQDCIVSLLSDKDDTKPKIAVMRPAKAQGNKDDKKSGTSDKATVNVADCLACSGCVTSAEAKLLEDQNVSEFMNILKQKRLTVVSISNQSCSSFACHLNCDLITIQRKLSGLFKHIGARFVMNSTISEYISLLETKYEFISRYKAKSDLPMIISHCPGWICYSEKSLNSSVLPLLSKVRSAQQLQGILIKTLTLEIYNQLLFLYKFRLSNSYRTNMNVKSTFTQNDNFVEQSDIFHVAIMPCHDKKLESTRSSLSLKSSDKNSSCPEVDIVLATSEVGEIIKLAGFNSLLDVPEAPLDNLWLNQNFQITKKHNLSLLITENYVSNQILNQFSWLIPSYFNSNSGGFCEYIIRSAIKELAGDHIDNKVQLPFNKLKNDILEAKYIKNNVELNYCLAYGFRAIQSISRKLNLQKNASQNTQYKQSVVNHVNYHLIEAMACPTGCVSGGGQILSQNDQNDDNSDLNKLRKNIKFIDEVQEALYKGINLNKNQEVILPDEIPIVNILYEYLIHIDKQIDRSSGLKLPFLRNDFVSINEVPTASSLKW

SEQ ID NO: 80 - protein, 469 aa, Kluyveromyces lactis
MSALLRDADLNDFISPGLACVKPAQPQKVEKKPSFEVEVGIESSEPEKVSISLQDCLACAGCITSSEEILLSKQSHKVFLEKWSELEELDERSLAVSISPQCRLSLADYYSMCLADLDRCFQNFMKTKFNAKYVVGTQFGRSISISRINATLKDRVPENEGPLLCSVCPGFVLYAEKTKPELIPHMLDVKSPQQITGNLLKQADPTCYHLSIMPCFDKKLEASREECEKEVDCVITPKQFVAMLGDLSIDFKSYMTEYDSSKELCPSGWDYKLHWLSNEGSSSGGYAYQYLLSLQSSNPESDIITIEGKNSDVTEYRLVSKSKGVIASSSEVYGFRNIQNLVRKLSQSASVKKRGIKVKRRGQSVLKSGETSEKTTKVLTADPAKTDFVEVMACPSGCINGGGLLNEEKNANRRKQLAQDLSLAYTKVHSVNIPDIVHAYDDKSNDFKYNLRVIEPSTSSDVVAVGNTW

SEQ ID NO: 81 - protein, 365 aa, Encephalitozoon cuniculi
MDALIRPPMSFFADLPKDNKKCIKIGSPLALSLSDCLACSGCVSADEAGALSEDLSFVLDLSPQTSFVLSPQSKINIFNLYREDGMEYREFEAVLSSFLRSKFNIHRIVDTSYLRSKIYEETYREYMATNHLIVSACPGVVTYIERTAPYLIGYLSRVKSPQQMAFSLVKGSRTVSVMPCQDKKLENGRDGVKFDFILTTRGFCKALDSLGFRRPARASGKSLCSMEEAETTQWNIGTSSGGYAEFILGKHCVVETREIRNGIKEHLLDDGRTISQITGLENSINYFKSSKTKGPRHKMTEIFLCKNGCIGGPGQERVNDVEMDIREYDRNGREQPRIFYSSPGLEEKRVFREVKAKRVDLRVDW

SEQ ID NO: 82 - protein, 127 aa, Tritrichomonas foetus
  misc_feature (85)..(85): Xaa can be any naturally occurring amino acid
MCIKACNSVAGQGVLKLVKVGNKKLVSTKSGKPLQETNCIKCGQCTLVCGPGALTQKDAIQTVSEVLKNPGDKVLVCQTAPAIRXNLADGLGMPAGSIITGKMVTALKMLGFKYVFDTNFGTDXTIG

SEQ ID NO: 83 - protein, 449 aa, Scenedesmus obliquus
MPEWQPGGRYAVSVRPPVNRRAVVAAERRRLVVRAAGPTAECDCPPAPAPKAPHWQQTLDELAKPKEQRKVMIAQIAPAVRVAIAETMGLNPGDVTVGQMVTGLRMLGFDYVFDTLFGADLTIMEEGTELRHRLQDHLEQHPNKEEPLPMFTSCCPGWVAMVEKSNPELIPYLSSCKSPQMMLGAVIKNYFAAEAGAKPEDICNVSVMPCVRKQGEADREWFNTTGAGGANVDHVMTTAELGKIFVERGIKLNDLQESPFDNPVGEGSGGGVLFGTTGGVMEAALRTVYEVVTQKPLDRIVFEDVRGLEGIKESTLHLTPGPTSPFKAFAGADGTGITLNIAVANGLGNAKKLIKQLAAGESKYDFIEVMACPGGCIGGGGQPRSADKQILQKRQAAMYDLDERAVIRRSHENPLIGALYEKFLGEPNGHKAHELLHTHYVAGGVPDEK

SEQ ID NO: 84 - protein, 477 aa, Anopheles gambiae
SRFSSALQLTDLDDFITPSQECIKPVKIETSKSKTGAKITIQEDGSYVQESSSGIQKLEKVEITLADCLACSGCITSAEGVLISQQSQEELLRVMNANNLAKLNNQRDEIKFVVFTVSQQPILSLARKYNLTPEDTFEHIAGYFKKLGADMVVDTKIADDLALIECRNEFIERYNTNRKLLPMLASSCPGWVCYAEKTHGNFILPYIATTRSPQQIMGVLVKQYLAKQLQTTGDRIYHVTVMPCYDKKLEASREDFFSEVENSRDVDCVITSIEIEQMLNSLDLPSLQLVERCAIDWPWPTVRPSAFVWGHESSGSGGYAEYIFKYAARKLFNVQLDTVAFKPLRNNDMREAVLEQNGQVLMRFAIANGFRNIQNMVQKLKRGKSTYDYVEIMACPSGCLNGGAQIRPEEGRAARELTAELECMYRSLPQSTPENDCVQTMYATFFDSEGDLNKRQSLLHTSYHQIEKINSALNIKW

SEQ ID NO: 85 - protein, 410 aa, Shewanella oneidensis
MTTTTYQPGEIQGLIKINASKCKGCDACKQFCPTHAINGASGAVHSIDEDKCLSCGQCLINCPFSAIEETHSALETVIKKLADKNTTVVGIIAPAVRVAIGEEFGLGTGELVTGKLYGAMNQAGFKIFDCNFAADLTIMEEGSEFIHRLHANVKGEANAGPLPQFTSCCPGWVRYLETRYPALLPNLSTAKSPQQMAGTVAKTYGAKVYQMQPENIFTVSVMPCTSKKLEASRPEFNSAWQYHQEHGANSPSYQDIDAVLTTREMAQLLKLLDIDLANTAEYQGDSLFSEYTGAGTIFGTTGGVMEAALRTAHKVLTGTEMAKLEFEPVRGLKGVKSASVSLFDTELNQDVTVNVAVVHDMGNNIEPVLRDVMAGTSPYHFIEVMNCAGGCVNGGGQPIEGKGSSWLGNI

SEQ ID NO: 86 - protein, 606 aa, Clostridium thermocellum
MAFVWRNVRSRPFPKKPNGRGCEKMQMVNVTIDNCKIQVPANYTVLEAAKQANIDIPTLCFLKDINEVGACRMCVVEVKGARSLQAACVYPVSEGLEVYTQTPAVREARKVTLELILSNHEKKCLTCVRSENCELQRLAKDLNVKDIRFEGEMSNLPIDDLSPSVVRDPNKCVLCRRCVSMCKNVQTVGAIDVTERGFRTTVSTAFNKPLSEVPCVNCGQCINVCPVGALREKDDIDKVWEALANPELHVVVQTAPAVRVALGEEFGMPIGSRVTGKMVAALSRLGFKKVFDTDTAADLTIMEEGTELINRIKNGGKLPLITSCSPGWIKFCEHNYPEFLDNLSSCKSPHEMFGAVLKSYYAQKNGIDPSKVFVVSIMPCTAKKFEAQRPELSSTGYPDVDVVLTTRELARMIKETGIDFNSLPDKQFDDPMGEASGAGVIFGATGGVMEAAIRTVGELLSGKPADKIEYTEVRGLDGIKEASIELDGFTLKAAVAHGLGNARKLLDKIKAGEADYHFIEIMACPGGCINGGGQPIQPSSVRNWKDIRCERAKAIYEEDESLPIRKSHENPKIKMLYEEFFGEPGSHKAHELLHTHYEKRENYPVK

SEQ ID NO: 87 - protein, 279 aa, Desulfitobacterium hafniense
MTMGQLRAALKHLGFYGMIEVALFADVLSLKEALEFDKHVQTDKDFVLTSCCCPIWVGMVKRVYDTLVPHISPSVSPMVACGRGIKRLHPDAKTVFIGPCIAKKAEAKEPDIRDAVDAVLTFHELKQIFEATDIEPSEMEDIPSEHSSTSGRIYARTGGVSKSISDTLNRIRPDKPVKIKSIQANGIKECKALLNDIMNNEIKANFYEGMGCPGGCVGGPKAIVDVDRGTEFVNKYGAEADALTPADNQHVLELLKQLGIDSVEELLGGESAAIFQRDF

SEQ ID NO: 88 - protein, 505 aa, C. reinhardtii
MALGLRAELRAGQAVACARRTNAPAHPAAVVPVLPSRGDKFFNLSQKVPSSQPARGSTIRVAATATDAVPHWKLALEELDKPKDGGRKVLIAQVAPAVRVAIAESFGLAPGAVSPGKLAAGLRALGFDQVFDTLFAADLTIMEEGTELLHRLKEHLEAHPHSDEPLPMFTSCCPGWVAMMEKSYPELIPFVSSCKSPQMMMGAMVKTYLSEKQGIPAKDIVMVSVMPCVRKQGEADREWFCVSEPGVRDVDHVITTAELGNIFKERGIILPELPDSDWDQPLGLGSGAGVLFGTTGGVMEAAVRTAYEIVTKEPLPRLNLSEVRGLDGIKEASVTLVPAPGSKFAELVAARLAHKVEEAAAAEAAAAVEGAVKPPIAYDGGQGFSTDDGKGGLKLRVAVANGLGNAKKLIGKMVSGEAKYDFVEIMACPAGCVGGGGQPRSTDKQITQKRQAALYDLDERNTLRRSHENEAVNQLYKEFLGEPLSHRAHELLHTHYVPGGAEADA

SEQ ID NO: 89 - protein, 505 aa, C. reinhardtii
MALGLRAELRAGQAVACARRTNAPAHPAAVVPVLPSRGDKFFNLSQKVPSSQPARGSTIRVAATATDAVPHWKLALEELDKPKDGGRKVLIAQVAPAVRVAIAESFGLAPGAVSPGKLAAGLRALGFDQVFDTLFAADLTIMEEGTELLHRLKEHLEAHPHSDEPLPMFTSCCPGWVAMMEKSYPELIPFVSSCKSPQMMMGAMVKTYLSEKQGIPAKDIVMVSVMPCVRKQGVADREWFCVSEPGVRDVDHVITTAELGNIFKERGIILPELPDSDWDQPLGLGSGAGVLFGTTGGVMEAAVRTAYEIVTKEPLPRLNLSEVRGLDGIKEASVTLVPAPGSKFAELVAARLAHKVEEAAAAEAAAAVEGAVKPPIAYDGGQGFSTDDGKGGLKLRVAVANGLGNAKKLIGKMVSGEAKYDFVEIMACPAGCVGGGGQPRSTDKQITQKRQAALYDLDERNTLRRSHENEAVNQLYKEFLGEPLSHRAHELLHTHYVPGGAEADA

SEQ ID NO: 90 - protein, 608 aa, T. maritima; identical to SEQ ID NO: 16

SEQ ID NO: 91 - protein, 497 aa, C. reinhardtii; identical to SEQ ID NO: 24

SEQ ID NO: 92 - protein, 581 aa, T. tencongensis; identical to SEQ ID NO: 28

SEQ ID NO: 93 - protein, 636 aa, N. frontalis; identical to SEQ ID NO: 29

SEQ ID NO: 94 - protein, 579 aa, C. thermocellum
MVNVTIDNCKIQVPANYTVLEAAKQANIDIPTLCFLKDINEVGACRMCVVEVKGARSLQAACVYPVSEGLEVYTQTPAVREARKVTLELILSNHEKKCLTCVRSENCELQRLAKDLNVKDIRFEGEMSNLPIDDLSPSVVRDPNKCVLCRRCVSMCKNVQTVGAIDVTERGFRTTVSTAFNKPLSEVPCVNCGQCINVCPVGALREKDDIDKVWEALANPELHVVVQTAPAVRVALGEEFGMPIGSRVTGKMVAALSRLGFKKVFDTDTAADLTIMEEGTELINRIKNGGKLPLITSCSPGWIKFCEHNYPEFLDNLSSCKSPHEMFGAVLKSYYAQKNGIDPSKVFVGSIMPCTAKKFEAQRPELSSTGYPDVDVVLTTRELARMIKETGIDFNSLPDKQFDDPMGEASGAGVIFGATGGVMEAAIRTVGELLSGKPADKIEYTEVRGLDGIKEASIELDGFTLKAAVAHGLGNARKLLDKIKAGEADYHFIEIMACPGGCINGGGQPIQPSSVRNWKDIRCERAKAIYEEDESLPIRKSHENPKIKMLYEEFFGEPGSHKAHELLHTHYEKRENYPV

SEQ ID NO: 95 - protein, 588 aa, B. thetaoimicron
MEEKQITLQIDGHFITVPEGSTILEAACKIGINIPTLCHIDLKGTCIKNNPASCRICVVEVAGRRNLAPACATRCTEGMVVKTSTLRVMNARKVVAELILSDHPNDCLTCPKCGNCELQTLALRFNIREMPFNGGELSPRKREVTSSIVRNMDKCIFCRRCESVCNDVQTVGALGAIRRGFNTTIAPAFDRMMKDSECTYCGQCVAVCPVGALTERDYTNRLLDDLADPDKIVIVQTAPAVRAALGEEFGLPPGTLVTGKMVYALRELGFDYVFDTDFAADLTIMEEGSEILNRLTRYLDGDKSVRLPILTSCCPAWVNFFEHHFPDMLDIPSTARSPQQMFGSIAKSYWAEKMGIPREKLVVVSIMPCLAKKYECDRDEFKVNGVPDVDYSISTRELARLIKRANIGFTLVLDSPFDNPMGESTGAGVIFGTTGGVMEAALRSVYEIYTGQPLKNVNFEQVRGLSGVRRATIDLNGFELKVGIAHGLGNARHLLEDIRNGHNEYHVIEIMACPGGCIGGGGQPLHHGNSDVLYARANALYREDANKPLRKSHDNPYIQKLYEEYLGKPLGEKSEMLLHTHYFNKSID

SEQ ID NO: 96 - protein, 585 aa, D. fructosovorans; identical to SEQ ID NO: 14

SEQ ID NO: 97 - protein, 606 aa, D. vulgaris; identical to SEQ ID NO: 9

SEQ ID NO: 98 - protein, 589 aa, T. vaginalis; identical to SEQ ID NO: 22

SEQ ID NO: 99 - protein, 1206 aa, Nyctotherus ovalis; identical to SEQ ID NO: 18

SEQ ID NO: 100 - protein, 468 aa, T vaginalis; identical to SEQ ID NO: 20

SEQ ID NO: 101 - protein, 582 aa, C. acetobutylicum; identical to SEQ ID NO: 23

SEQ ID NO: 102 - protein, 574 aa, Clostridium pasteurianum; identical to SEQ ID NO: 1

SEQ ID NO: 103 - protein, 421 aa, Desulfovibrio vulgaris; identical to SEQ ID NO: 2

SEQ ID NO: 104 - protein, 449 aa, Trichomonas vaginalis; identical to SEQ ID NO: 21

SEQ ID NO: 105 - protein, 645 aa, T. maritima; identical to SEQ ID NO: 17

SEQ ID NO: 106 - protein, 369 aa, T vaginalis; identical to SEQ ID NO: 45

SEQ ID NO: 107 - protein, 476 aa, R. norvegicus
MASPFSGALQLTDLDDFIGPSQSCIKPVTVAKKPGSGIAKIHIEDDGSYFQVNPDGRSQKLEKAKVSLNDCLACSGCVTSAETILITQQSHEELRKVLDANKVAAPGQQRLVVVSVSPQSRASLAARFQLDSTDTARKLTSFFKKIGVHFVFDTAFARNFSLLESQKEFVQRFREQANSREALPMLASACPGWICYAEKTHGNFILPYISTARSPQQVMGSLIKDFFAQQQLLTPDKIYHVTVMPCYDKKLEASRPDFFNQEYQTRDVDCVLTTGEVFRLLEEEGVSLSELEPVPLDGLTRSVSAEEPTSHRGGGSGGYLEHVFRHAAQELFGIHVADVTYQPMRNKDFQEVTLEREGQVLLRFAVAYGFRNIQNLVQKLKRGRCPYHYVEVMACPSGCLNGGGQLKAPDTEGRELLQQVERLYSMVRTEAPEDAPGVQELYQHWLQGEDSERASHLLHTQYHAVEKINSGLSIRW

SEQ ID NO: 108 - protein, 525 aa, S. cerevisiae [sequence omitted]

SEQ ID NO: 109 - protein, 572 aa, C. perfringens; identical to SEQ ID NO: 11

SEQ ID NO: 110 - protein, 572 aa, C. perfringens; identical to SEQ ID NO: 10

SEQ ID NO: 111 - protein, 494 aa, C. tetani; identical to SEQ ID NO: 71

SEQ ID NO: 112 - protein, 448 aa, C. tetani; identical to SEQ ID NO: 69

SEQ ID NO: 113 - protein, 261 aa, Pyrococcus furiosus
MGKVRIGFYALTSCYGCQLQLAMMDELLQLIPNAEIVCWFMIDRDSIEDEKVDIAFIEGSVSTEEEVELVKKIRENAKIVVAVGACAVQGGVQSWSEKPLEELWKKVYGDAKVKFQPKKAEPVSKYIKVDYNIYGCPPEKKDFLYALGTFLIGSWPEDIDYPVCLECRLNGHPCILLEKGEPCLGPVTRAGCNARCPGFGVACIGCRGAIGYDVAWFDSLAKVFKEKGMTKEEIIERMKMFNGHDERVEKMVEKIFSGGEQ

SEQ ID NO: 114 - protein, 252 aa, Escherichia coli
MSPVLTQHVSQPITLDEQTQKMKRHLLQDIRRSAYVYRVDCGGCNACEIEIFAAITPVFDAERFGIKVVSSPRHADILLFTGAVTRAMRMPALRAYESAPDHKICVSYGACGVGGGIFHDLYSVWGGSDTIVPIDVWIPGCPPTPAATIHGFAVALGLLQQKIHAVDYRDPTGVTMQPLWPQIPPSQRIAIEREARRLAGYRQGREICDRLLRHLSDDPTGNRVNTWLRDADDPRLNSIVQQLFRVLRGLHD

SEQ ID NO: 115 - protein, 236 aa, Methanothermobacter thermautotrophicus
MAEENAKPRIGYIHLSGCTGDAMSLTENYDILAELLTNMVDIVYGQTLVDLWEMPEMDLALVEGSVCLQDEHSLHELKELREKAKLVCAFGSCAQTGCFTRYSRGGQQAQPSHESFVPIADLIDVDLAIPGCPPSPEIIAKAVVALLNNDMEYLQPMLDLAGYTEACGCDLQTKVVNQGLCTGCGTCAMACQTRALDMTNGRPELNSDRCIKCGICYVQCPRSWWPEEQIKKELGL

SEQ ID NO: 116 - protein, 259 aa, Methanosarcina barkeri
MANKIKLGHVHLSGCTGCLVSVADNYQGFLKILDDYADLVYCLTLADVRHIPEMDVALVEGSVCIQDRESVEDIKETRKKSRIVVALGSCASYGNITRFCRGGQHNHPQHESYLPIGDLIDVDVYIPGCPPSPELIRNVAIMAYLLLEGNEEQKDLAGRYLKPLMDLAKRGTTGCFCDLMDDVINQGLCIGCGICAASCPVRAITHEFGKPQGDLNLCIKCGSCYGACPRSFFNPDVISEFESINEIIAGALKEGEKDD

SEQ ID NO: 117 - protein, 142 aa, Rhodospirillum rubrum
MNFLSRMSKKSPWLYRINAGSCNGCDVELATTACIPRYDVERLGCQYCGSPKHADIVLVTGPLTARVKDKVLRVYEEIPDPKVTVAIGVCPISGGVFRESYSIVGPIDRYLPVDVNVPGCPPRPQAIIEGIAKAIEIWAGRI

SEQ ID NO: 118 - protein, 428 aa, Pyrococcus furiosus
MKNLYLPITIDHIARVEGKGGVEIIIGDDGVKEVKLNIIEGPRFFEAITIGKKLEEALAIYPRICSFCSAAHKLTALEAAEKAVGFVPREEIQALREVLYIGDMIESHALHLYLLVLPDYRGYSSPLKMVNEYKREIEIALKLKNLGTWMMDILGSRAIHQENAVLGGFGKLPEKSVLEKMKAELREALPLAEYTFELFAKLEQYSEVEGPITHLAVKPRGDAYGIYGDYIKASDGEEFPSEKYRDYIKEFVVEHSFAKHSHYKGRPFMVGAISRVINNADLLYGKAKELYEANKDLLKGTNPFANNLAQALEIVYFIERAIDLLDEALAKWPIKPRDEVEIKDGFGVSTTEAPRGILVYALKVENGRVSYADIITPTAFNLAMMEEHVRMMAEKHYNDDPERLKILAEMVVRAYDPCISCSVHVVRL

SEQ ID NO: 119 - protein, 555 aa, Escherichia coli
MNVNSSSNRGEAILAALKTQFPGAVLDEERQTPEQVTITVKINLLPDVVQYLYYQHDGWLPVLFGNDERTLNGHYAVYYALSMEGAEKCWIVVKALVDADSREFPSVTPRVPAAVWGEREIRDMYGLIPVGLPDQRRLVLPDDWPEDMHPLRKDAMDYRLRPEPTTDSETYPFINEGNSDARVIPVGPLHITSDEPGHFRLFVDGEQIVDADYRLFYVHRGMEKLAETRMGYNEVTFLSDRVCGICGFAHSVAYTNSVENALGIEVPQRAHTIRSILLEVERLHSHLLNLGLSCHFVGFDTGFMQFFRVREKSMTMAELLIGSRKTYGLNLIGGVRRDILKEQRLQTLKLVREMRADVSELVEMLLATPNMEQRTQGIGILDRQIARDLRFDHPYADYGNIPKTLFTFTGGDVFSRVMVRVKETFDSLAMLEFALDNMPDTPLLTEGFSYKPHAFALGFVEAPRGEDVHWSMLGDNQKLFRWRCRAATYANWPVLRYMLRGNTVSDAPLIIGSLDPCYSCTDRVTLVDVRKRQSKTVPYKEIERYGIDRNRSPLK

SEQ ID NO: 120 - protein, 405 aa, Methanothermobacter thermautotrophicus
MSERIVISPTSRQEGHAELVMEVDDEGIVTKGRYFSITPVRGLEKIVTGKAPETAPVIVQRICGVCPIPHTLASVEAIDDSLDIEVPKAGRLLRELTLAAHHVNSHAIHHFLIAPDFVPENLMADAINSVSEIRKNAQYVVDMVAGEGIHPSDVRIGGMADNITELARKRLYARLKQLKPKVDEHVELMIGLIEDKGLPKGLGVHNQPTLASHQIYGDRTKFDLDRFTEVMPESWYDDPEIAKRACSTIPLYDGRNVEVGPRARMVEFQGFKERGVVAQHVARALEMKTALARAIEILDELDTSAPVRADFDERGTGKLGVGAIEGPRGLDVHMAQVENGKIQFYSALVPTTWNIPTMGPATEGFHHEYGPHVIRAYDPCLSCATHVMVVDDEDRSVIRDEMVRL

SEQ ID NO: 121 - protein, 456 aa, Methanosarcina barkeri
MTKVVEISPTTRHEGHSKLTLKVNDEGIVERGDWLSTTPVRGIEKLAIGKTMDQVPKIASRVCGICPIAHTLAGIEAMEASIGCEIPKDAKLLRVILHAANRLHSHALHNILILPDFYIPDTETKINPFSKEQPLRSVAVRIFRIREIAQTIGAVAGGEAIHPSNPRVGGMYRNVSSRAKQKIADLAKEGLVLAHEQMEFMIEVIRNMQDREFVEVAGKQIPLPKTLGYHNQGVMATAPMYGSSSLDEKPMWDFTRWRETRPWDWYMSEETIDLEDSSYPIGGTTKVGTKVNPRMEACNTVPTYDGQPVEVGPRARLATFKHFTEKGTFAQHIARQMEYTDCYYTILNCLENLDTSGKVLADTIPLGNGSMGWAANEAPRGTDVHLARVKDGKVLRYEMLVPTTWNFPTCSRALTGAPWQIAEMVIRAYDPCVSCATHMIVVNEEDRIVAQKLMQW

SEQ ID NO: 122 - protein, 361 aa, Rhodospirillum rubrum
MSTYTIPVGPLHVALEEPMYFRIEVDGEKVVSVDITAGHVHRGIEYLATKRNIYQNIVLTERVCSLCSNSHPQTYCMALESITGMVVPPRAQYLRVIADETKRVASHMFNVAILAHIVGFDSLFMHVMEAREIMQDTKEAVFGNRMDIAAMAIGGVKYDLDKDGRDYFIGQLDKLEPTLRDEIIPLYQTNPSIVDRTRGIGVLSAADCVDYGLMGPVARGSGHAYDVRKQAPYAVYDRLDFEMALGEHGDVWSRAMVRWQEALTSIGLIRQCLRDMPDGPTKAGPVPPIPAGEAVAKTEAPRGELIYYLKTNGTDRPERLKWRVPTYMNWDALNVMMAGARISDIPLIVNSIDPCISCTER

SEQ ID NO: 123 - protein, 505 aa, Artificial sequence
  Synthetic sequence
MALGLLAELRAGQAVACARRTNAPAHPAAVVPCLPSRAGKFFNLSQKVPSSQSARGSTIRVAATATDAVPHWKLALEELDKPKDGGRKVLIAQVAPAVRVAIAESFGLAPGAVSPGKLATGLRALGFDQVFDTLFAADLTIWEEGTELLHRLKEHLEAHPHSDEPLPMFTSCCPGWVAMMEKSYPELIPFVSSCKSPQMMMGAMVKTYLSEKQGIPAKDIVMVSVMPCVRKQGEADREWFCVSEPGVRDVDHVITTAELGNIFKERGINLPELPDSDWDQPLGLGSGAGVLFGTTGGVMEAALRTAYEIVTKEPLPRLNLSEVRGLDGIKEASVTLVPAPGSKFAELVAERLAHKVEEAAAAEAAAAVEGAVKPPIAYDGGQGFSTDDGKGGLKLRVAVANGLGNAKKLIGKMVSGEAKYDFVEIMACPAGCVGGGGQPRSTDKQITQKRQAALYDLDERNTLRRSHENEAVNQLYKEFLGEPLSHRAHELLHTHYVPGGAEADA

SEQ ID NO: 124 - protein, 19 aa, Artificial sequence
  Synthetic sequence
GAGVIFGATGGVMEAALRT

SEQ ID NO: 125 - protein, 19 aa, Artificial sequence
  Synthetic sequence
GGGAIFCATGGVMEAAVRS

SEQ ID NO: 126 - protein, 19 aa, Artificial sequence
  Synthetic sequence
GGATIFGVTGGVMEAALRF

SEQ ID NO: 127 - protein, 19 aa, Artificial sequence
  Synthetic sequence
GAGAIFGATGGVMEAALRS

SEQ ID NO: 128 - protein, 19 aa, Artificial sequence
  Synthetic sequence
GAGAIFGATGGVMEAAIRS

SEQ ID NO: 129 - protein, 19 aa, Artificial sequence
  Synthetic sequence
GAAVIFGVTGGVMEAALRT

SEQ ID NO: 130 - protein, 19 aa, Artificial sequence
  Synthetic sequence
GAGQIFAATGGVMEAASRT

SEQ ID NO: 131 - protein, 19 aa, Artificial sequence
  Synthetic sequence
GAAVIFGTTGGVMEAALRT

SEQ ID NO: 132 - protein, 19 aa, Artificial sequence
  Synthetic sequence
GAAPIFGVTGGVIEAALRT

SEQ ID NO: 133 - protein, 19 aa, Artificial sequence
  Synthetic sequence
GAGVIFGTTGGVMEAALRS

SEQ ID NO: 134 - protein, 19 aa, Artificial sequence
  Synthetic sequence
GAGVIFGATGGVMEAAIRT

SEQ ID NO: 135 - protein, 19 aa, Artificial sequence
  Synthetic sequence
SAGNLFGVTGGVMEAAIRT

SEQ ID NO: 136 - protein, 19 aa, Artificial sequence
  Synthetic sequence
GAGAIFGATGGVMEAALRT

SEQ ID NO: 137 - protein, 19 aa, Artificial sequence
  Synthetic sequence
GAGVLFGTTGGVMEAALRT

SEQ ID NO: 138 - protein, 19 aa, Artificial sequence
  Synthetic sequence
GAAALFGVTGGVMEAALRT

SEQ ID NO: 139 - protein, 19 aa, Artificial sequence
  Synthetic sequence
GAGVLFGTTGGVMEAAVRT

SEQ ID NO: 140 - protein, 19 aa, Artificial sequence
  Synthetic sequence
GAGTIFGTTGGVMEAALRT

SEQ ID NO: 141 - protein, 19 aa, Artificial sequence
  Synthetic construct
GGGVLFGTTGGVMEAALRT

SEQ ID NO: 142 - protein, 5 aa, Artificial sequence
  Synthetic construct
TIMEE

SEQ ID NO: 143 - protein, 5 aa, Artificial sequence
  Synthetic construct
TIVEE

SEQ ID NO: 144 - protein, 5 aa, Artificial sequence
  Synthetic sequence
TIWEE

SEQ ID NO: 145 - protein, 5 aa, Artificial sequence
  Synthetic sequence
TICEE

SEQ ID NO: 146 - protein, 5 aa, Artificial sequence
  Synthetic sequence
VIMEE

SEQ ID NO: 147 - protein, 5 aa, Artificial sequence
  Synthetic sequence
TARLE

SEQ ID NO: 148 - DNA, 260 bp, Chlamydomonas reinhardtii
gcagttgggtcaggggctggcgacgcgctgctgacgcgcaagtgaatggcccaacaagtcgcctcgcggtcgctgtcggcgccaaacccgcagctgcatccaccagattcacttgttagatcgacctaggttgcgggaccggaggcggctcgctgtgcaagcgcggtgacctcgtacggcggcatggatcgccatctcgattcgcgcggcagaatcgggccccgcgcacatttaagccgcgggcgagactcatttcgtta

SEQ ID NO: 149 - DNA, 1181 bp, Artificial sequence
  Synthetic sequence
gccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaagagaagtcactcaacatcttaaaatggccaggtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcaggatggccaagctgaccagcgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactaaccgacgtcgacccactctagaggatcgatccccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttgaagcttaattaactcgagggggggcccggtacc

SEQ ID NO: 150 - DNA, 260 bp, Artificial sequence
  Synthetic sequence
ccgacgtcgacccactctagaggatcgatccccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttg

SEQ ID NO: 151 - DNA, 520 bp, Artificial sequence
  Synthetic sequence
ccgacgtcgacccactctagaggatcgatccccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttggcagttgggtcaggggctggcgacgcgctgctgacgcgcaagtgaatggcccaacaagtcgcctcgcggtcgctgtcggcgccaaacccgcagctgcatccaccagattcacttgttagatcgacctaggttgcgggaccggaggcggctcgctgtgcaagcgcggtgacctcgtacggcggcatggatcgccatctcgattcgcgcggcagaatcgggccccgcgcacatttaagccgcgggcgagactcatttcgtta

SEQ ID NO: 152 - DNA, 30 bp, Artificial sequence
  Synthetic sequence
atccgtagttatccttatggccatcttagc

SEQ ID NO: 153 - DNA, 30 bp, Artificial sequence
  Synthetic sequence
cgtgcatcgattaacagcttctggacctga

SEQ ID NO: 154 - DNA, 30 bp, Artificial sequence
  Synthetic sequence
ttaaacgtcgtacgtccaagtataactaag

SEQ ID NO: 155 - DNA, 30 bp, Artificial sequence
  Synthetic sequence
aatctgatacatgctattcagatcttacaa

SEQ ID NO: 156 - DNA, 30 bp, Artificial sequence
  Synthetic sequence
tcttccatcgtaaatctagcatcgattagc

SEQ ID NO: 157 - DNA, 30 bp, Artificial sequence
  Synthetic sequence
atctgtaataatctagtcgaggcattcaag

SEQ ID NO: 158 - DNA, 30 bp, Artificial sequence
  Synthetic sequence
aactggcttaaatcgttaacaatcgtgtga

SEQ ID NO: 159 - DNA, 30 bp, Artificial sequence
  Synthetic sequence
gatttaacataactgtcgattaccgtgcga

SEQ ID NO: 160 - DNA, 30 bp, Artificial sequence
  Synthetic sequence
tatgcttgacaatcgtaatcctggtgacaa

SEQ ID NO: 161 - DNA, 30 bp, Artificial sequence
  Synthetic sequence
taacaagaatctggctaatcaatcgatgca

SEQ ID NO: 162 - DNA, 30 bp, Artificial sequence
  Synthetic sequence
gtagtcggaatagttactaacgaggattcg

SEQ ID NO: 163 - DNA, 30 bp, Artificial sequence
  Synthetic sequence
aaatgtctactcgactagtaaatcgtaact

SEQ ID NO: 164 - DNA, 290 bp, Artificial sequence
  Synthetic sequence
gcagttgggtcaggggctggcgacgcgctgctgacgcgcaagtgaatggcccaacaagtcgcctcgcggtcgctgtcggcgccaaacccgcagctgcatccaccagattcacttgttagatcgacctaggttgcgggaccggaggcggctcgctgtgcaagcgcggtgacctcgtacggcggcatggatcgccatctcgattcgcgcggcagaatcgggccccgcgcacatttaagccgcgggcgagactcatttcgttaatccgtagttatccttatggccatcttagc

SEQ ID NO: 165 - DNA, 580 bp, Artificial sequence
  Synthetic sequence
cgtgcatcgattaacagcttctggacctgaccgacgtcgacccactctagaggatcgatccccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttggcagttgggtcaggggctggcgacgcgctgctgacgcgcaagtgaatggcccaacaagtcgcctcgcggtcgctgtcggcgccaaacccgcagctgcatccaccagattcacttgttagatcgacctaggttgcgggaccggaggcggctcgctgtgcaagcgcggtgacctcgtacggcggcatggatcgccatctcgattcgcgcggcagaatcgggccccgcgcacatttaagccgcgggcgagactcatttcgttattaaacgtcgtacgtccaagtatgactaag

SEQ ID NO: 166 - DNA, 566 bp, Artificial sequence
  Synthetic sequence
aatctgatacatgctattcagatcttacaaccgacgtcgacccactctagaggatcgatccccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttggcagttgggtcaggggctggcgacgcgctgctgacgcgcaagtgaatggcccaacaagtcgcctcgcggtcgctgtcggcgccaaacccgcagctgcatccaccagattcacttgttagatcgacctaggttgcgggaccggaggcggctcgctgtgcaagcgcggtgacctcgtacggcggcatggatcgccatctcgattcgcgcggcagaatcgggccccgcgcacatttaagccgcgggcgatcttccatcgtaaatctagcatcgattagc

SEQ ID NO: 167 - DNA, 290 bp, Artificial sequence
  Synthetic sequence
atctgtaataatctagtcgaggcattcaagccgacgtcgacccactctagaggatcgatccccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttg

SEQ ID NO: 168 - DNA, 1181 bp, Artificial sequence; identical to SEQ ID NO: 149
  Synthetic sequence

SEQ ID NO: 169 - DNA, 290 bp, Artificial sequence
  Synthetic sequence
gcagttgggtcaggggctggcgacgcgctgctgacgcgcaagtgaatggcccaacaagtcgcctcgcggtcgctgtcggcgccaaacccgcagctgcatccaccagattcacttgttagatcgacctaggttgcgggaccggaggcggctcgctgtgcaagcgcggtgacctcgtacggcggcatggatcgccatctcgattcgcgcggcagaatcgggccccgcgcacatttaagccgcgggcgagactcatttcgttaaactggcttaaatcgttaacaatcgtgtga

SEQ ID NO: 170 - DNA, 566 bp, Artificial sequence
  Synthetic sequence
gatttaacataactgtcgattaccgtgcgaccgacgtcgacccactctagaggatcgatccccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttggcagttgggtcaggggctggcgacgcgctgctgacgcgcaagtgaatggcccaacaagtcgcctcgcggtcgctgtcggcgccaaacccgcagctgcatccaccagattcacttgttagatcgacctaggttgcgggaccggaggcggctcgctgtgcaagcgcggtgacctcgtacggcggcatggatcgccatctcgattcgcgcggcagaatcgggccccgcgcacatttaagccgcgggcgatatgcttgacaatcgtaatcctggtgacaa

SEQ ID NO: 171 - DNA, 290 bp, Artificial sequence
  Synthetic sequence
taacaagaatctggctaatcaatcgatgcaccgacgtcgacccactctagaggatcgatccccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttg

SEQ ID NO: 172 - DNA, 381 bp, Chlamydomonas reinhardtii
atggccatggctatgcgctccaccttcgccgcccgcgttggcgctaagcccgctgtccgcggtgctcgccccgccagccgcatgagctgcatggcctacaaggtcaccctgaagaccccttcgggcgacaagaccattgagtgccccgctgacacctacatcctggacgctgctgaggaggccggcctggacctgccctactcttgccgcgctggtgcttgctccagctgcgccggcaaggtcgctgccggcaccgtcgaccagtcggaccagtccttcctggacgatgcccagatgggcaacggcttcgtgctgacctgcgtggcctaccccacctcggactgcaccatccagacccaccaggaggaggccctgtactaa

SEQ ID NO: 173 - DNA, 1494 bp, Chlamydomonas reinhardtii
atgtcggcgctcgtgctgaagccctgcgcggccgtgtctattcgcggcagctcctgcagggcgcggcaggtcgccccccgcgctccgctcgcagccagcaccgtgcgtgtagcccttgcaacacttgaggcgcccgcacgccgcctaggcaacgtcgcttgcgcggctgccgcacccgctgcggaggcgcctttgagtcatgtccagcaggcgctcgccgagcttgccaagcccaaggacgaccccacgcgcaagcacgtctgcgtgcaggtggctccggccgttcgtgtcgctattgccgagaccctgggcctggcgccgggcgccaccacccccaagcagctggccgagggcctccgccgcctcggctttgacgaggtgtttgacacgctgtttggcgccgacctgaccatcatggaggagggcagcgagctgctgcaccgcctcaccgagcacctggaggcccacccgcactccgacgagccgctgcccatgttcaccagctgctgccccggctggatcgctatgctggagaaatcttacccggacctgatcccctacgtgagcagctgcaagagcccccagatgatgctggcggccatggtcaagtcctacctagcggaaaagaagggcatcgcgccaaaggacatggtcatggtgtccatcatgccctgcacgcgcaagcagtcggaggctgaccgcgactggttctgtgtggacgccgaccccaccctgcgccagctggaccacgtcatcaccaccgtggagctgggcaacatcttcaaggagcgcggcatcaacctggccgagctgcccgagggcgagtgggacaatccaatgggcgtgggctcgggcgccggcgtgctgttcggcaccaccggcggtgtcatggaggcggcgctgcgcacggcctatgagctgttcacgggcacgccgctgccgcgcctgagcctgagcgaggtgcgcggcatggacggcatcaaggagaccaacatcaccatggtgcccgcgcccgggtccaagtttgaggagctgctgaagcaccgcgccgccgcgcgcgccgaggccgccgcgcacggcacccccgggccgctggcctgggacggcggcgcgggcttcaccagcgaggacggcaggggcggcatcacactgcgcgtggccgtggccaacgggctgggcaacgccaagaagctgatcaccaagatgcaggccggcgaggccaagtacgactttgtggagatcatggcctgccccgcgggctgtgtgggcggcggcggccagccccgctccaccgacaaggccatcacgcagaagcggcaggcggcgctgtacaacctggacgagaagtccacgctgcgccgcagccacgagaacccgtccatccgcgagctgtacgacacgtacctcggagagccgctgggccacaaggcgcacgagctgctgcacacccactacgtggccggcggcgtggaggagaaggacgagaagaagtga

SEQ ID NO: 174 - DNA, 1725 bp, Clostriduim pasteuranum
atgaaaacaataattataaatggtgtacagtttaatactgatgaagacactactatattaaaatttgcacgagacaacaatattgatatatctgcactgtgttttttaaataattgtaataatgacataaataagtgtgaaatatgtactgtagaggtagagggtactggattagtaacagcctgtgatacattaattgaggatggtatgattataaacacaaattccgatgctgtcaacgaaaaaattaaatctagaatatctcaattattagacatacatgaattcaaatgtggtccttgcaatagaagagaaaactgtgaattcttaaaacttgttataaaatataaagcaagagcttctaaaccatttttacctaaagataagactgaatatgtagatgaaagaagtaaatcattaactgtagataggacaaaatgcttattatgtggaagatgtgttaatgcctgtggaaaaaatactgaaacctatgcaatgaaatttttaaacaaaaatggtaaaactataattggagcagaggatgaaaaatgctttgatgatactaattgtctattatgtggtcaatgtataatcgcctgtccagtagcagcattatcggaaaaatcacacatggatagagtaaaaaatgccttaaatgcccctgaaaaacatgtaatagtagctatggctccatctgtcagagcttctataggtgaactttttaatatgggatttggcgttgacgtaacaggaaaaatttatactgctttaagacagcttggatttgataaaatattcgatataaacttcggagcagatatgacaattatggaagaggctacagaattagttcaaagaatagagaataatggacctttcccaatgtttacatcttgctgcccaggttgggtaagacaagctgaaaattattatcctgaattactaaataatctttcatcagctaaatcacctcaacaaatttttggtactgctagtaaaacttattatccttctatatctggtcttgacccaaagaatgtatttactgtaacagttatgccctgtacttcaaaaaaatttgaagcagatagaccacaaatggaaaaagacggcctaagagatatagatgctgttataactactcgagaattagcaaaaatgattaaagatgctaaaataccatttgctaaacttgaagatagcgaagcagaccctgctatgggagaatacagcggtgctggtgccatatttggtgcaactggcggagttatggaagcagctttaagaagtgcaaaagactttgctgaaaacgctgaacttgaagatatagaatataagcaagttagaggattaaatggtataaaagaagctgaagtagaaataaataacaacaaatataatgtagctgttataaatggtgcttcaaatttatttaagtttatgaaatctggtatgattaacgaaaaacaatatcatttcatagaagtaatggcttgtcatggaggatgtgtaaatggtggtggacagcctcatgtaaacccaaaagatttagaaaaagtagacataaaaaaagtaagagcttctgtattgtataatcaggatgaacatctttccaagagaaaatctcatgaaaatactgcattagttaaaatgtatcaaaattattttggcaaaccaggtgaaggtcgtgcccatgaaatattacactttaaatataaaaaataa

SEQ ID NO: 175 - DNA, 1265 bp, Desulfovibrio vulgaris
atgagccgtaccgtcatggagcgcatcgaatatgagatgcacactccggaccccaaggccgatccggacaagctccacttcgtccagatcgacgaggcaaagtgcataggctgcgacacctgttcgcagtactgccccaccgccgccatcttcggcgaaatgggcgaaccgcactccattccccacatcgaggcgtgcatcaactgcggccagtgcctcacgcactgccccgagaacgccatctacgaggcacagtcgtggtgcctgaagtcgagaagaagctgaaggacggcaaggtgaaatgcatcgccatgcccgcccccgccgtgcgctatgcactgggcgacgccttcggcatgcccgtcggttccgtcaccaccggcaagatgctcgcggccctgcagaagctcggcttcgctcattgctgggacaccgagttcaccgctgacgtgaccatctgggaagaggggtccgagttcgtggaacgcctcaccaagaagagcgacatgccgctgccgcagttcacctcgtgctgccccggctggcagaagtatgccgagacctactaccccgaactgctgccgcacttctccacgtgcaagtcgcccatcggcatgaacggcgcactggcgaagacctacggcgcagagcggatgaagtacgaccccaagcaggtctacaccgtctccatcatgccctgcatcgcaaagaagtacgaagggttgcgtcccgaactgaagtccagcggcatgcgcgacatcgacgccacgctgaccacccgtgagctggcctacatgatcaagaaggccggtatcgacttcgcgaaactccccgacggcaagcgtgacagcctcatgggtgaatccaccggcggtgccaccatcttcggcgtcaccggcggcgtcatggaagcggcactccgcttcgcctacgaagccgtcaccggcaagaagcccgacagctgggacttcaaggccgtgcgcggtcttgatggcatcaaggaagccaccgtcaacgtcggcggtaccgacgtcaaggtcgccgtggtgcacggggccaagcggttcaagcaggtctgcgacgatgtgaaggcgggcaagtcgccctatcacttcatcgaatacatggcctgccccggcggctgcgtctgtggcggcggtcagcccgtcatgcccggcgtgctcgaagccatggaccgcaccaccacccgcctttacgcgggcctgaagaagcgcctcgccatggcgagcgccaacaaggcatag

SEQ ID NO: 176 - DNA, 1407 bp, Entamoeba histolytica
atgccacctaaaccatcacatacactcaccggacatgaccataaccatagtattcaatttgattggtctaaatgcatgggttgtggaatgtgtgctactaaatgtacttttggggtgttagtaaaacaaccaccaaaaattccaccatttgttcagcctaatagagaaaaactctctcaagaaaataccgacaagacaagagtacttattgatgagtctgaatgtactgggtgtggtcaatgttctttggtttgtaactttggttctattacaccaatagaccatcttgttgatacttttaaagctaaagaagctggaaagaagcttgttgctatgattgcaccttcaactcgtttaggtgttgctgaggctatgggaatgcctattggaagtacagctatggctcagttagttcattgtttaagacttattggatttgattatgtatttgatgttgatgctggagctgataagacaacaatggatgattatgccgaagttattgaaatgaaaaaagaaggaaaaggacctgctattacttcctgttgtcctgcttggattgaacttgttgaaaaagaatatcctgacttaattccaaacgtctctactgcccgttcaccaattggatgtttagctggttgtattaaaagaggatgggcaaaggatgtaggaattgcagtagaagatctttacactgttggaataatgccttgtattgctaaaaaaacagagtctcaaagacaacaaattcatcaagactatgatgcttcatgtacttcaaatgaaattgctgcttatttcaaaaaacatcttccacctgaagaatgtaaatttacacaagaaagagaagaagcacttgctaaaactgaagatggtcaatgtgatttaccatttagacgtatttctggtggttctaatatttttggaaagactggaggagtttgtgaaactgtattgagagtaattgcacgtaatgcaggagttgattggaacagttgtactgttaacaaggaagaaacttttaaacatgctgcaagtggatcaacaatgacaaatctttctgttgatattggtggaactattatcacaggtgctgtttgtcatggtggttatgctattagacatgcttgtgaacttattagaaaaggagagttaaaagttgatgttgttgaaatgatggcatgtgttggaggttgtcttggaggagcaggtcaaccaaaaattccaccagcaaagaaacttgagatggataagagaagagtaatgttagatattttagatcaacaaactgatattagagctgctaatgaaaatactgatgttcttggatggattgataaacattttgatcatcaaggtgcacatcagcatcttcacacatattttactcccagatatcaaaactaa

SEQ ID NO: 177 - DNA, 1350 bp, Scenedesmus obliquus
atgcctgagtggcaaccgggaggtcggtatgctgtttctgtccgcccgccagtgaacaggcgggctgtggtggcagcagagcgcaggcgccttgttgtgcgggcagctggcccaacagcagaatgtgattgcccaccagctcccgcgcccaaggccccgcactggcagcagacgctagatgagctagccaagcctaaggagcagcgcaaggtgatgatcgcccagatcgcaccagcagtgcgcgtggctattgcagagaccatgggactcaaccctggggatgtgacagttggccagatggtgaccggcctgcgcatgctgggctttgattatgtgtttgacacgctgtttggtgctgacctcaccatcatggaggagggcacagagctacggcacaggcttcaggaccacctggagcagcaccccaacaaggaggagccgctgcccatgttcaccagctgctgccctggctgggtggccatggtggagaagtccaaccccgagctcatcccctacctgtcttcctgcaagtcgccccagatgatgctgggcgcagtcatcaagaactacttcgctgccgaggccggcgccaagcctgaggacatctgcaacgtgagcgtgatgccctgcgtgcgcaagcagggcgaggctgaccgcgagtggttcaacaccacaggggctggcggcgcgaacgtggaccacgtcatgacaactgcagagctgggcaagatctttgtggagcgcggaatcaagctgaacgacctgcaggagtcgccctttgacaaccccgtcggcgagggcagcggcggcggcgtgctgttcggcaccactggaggcgtgatggaggcggcgctgcgcaccgtgtacgaagtggtcacacagaagcctttggaccgcatcgtctttgaggacgtgcgcggcctggagggcatcaaggagtccacgctgcacctcaccccaggccccaccagccccttcaaggcctttgcaggcgcagacggcaccggcatcaccctcaacatcgcggtcgccaacggcctcggcaatgccaagaagctcatcaagcagctggctgcaggcgagagcaagtacgacttcatcgaggtcatggcctgccccggcggctgcatcggcggcggcggccagccgcgcagcgcggacaagcagatcctgcagaagcgccaggcggccatgtacgacctggacgagcgcgcggtgatccggcgcagccacgagaacccgctgattggcgcgctgtatgagaagttcctgggcgagcccaacggccacaaggcgcacgagctgctgcacacgcactacgtggccggcggcgtgcccgatgagaagtga

SEQ ID NO: 178 - DNA, 1311 bp, Chlorella fusca
atgtgttgccccgtggttgcaagtaggcacgcagggcgtgcaaggcatgttgctgtccgtgcagcagggccaacatctgagtgtgattgtcctccaacacctcaggccaagctgcctcactggcagcaggctctggatgagctcgccaagcccaaggagagcaggaggttgatgatcgcgcaaatcgcctccgctgttcgtgtcgctattgctgagaccattggcttggccccaggagatgtcaccattgggcagctcgtgactgggctgcgtatgcttggctttgattatgtctttgacaccctgtttggtgctgacctgaccattatggaggagggaacggagctgctgcatcgcctgcaggaccatctggagcagcaccccaacaaggaggagccactgcccatgttcaccagttgctgcccaggctgggttgccatggttgaaaagagcaatcctgagctcatcccctacctgtcatcttgcaagtcgcctcagatgatgcttggggccgttatcaagaactactatgcacagcaggttggagtgcagcccagtgacatctgcaacgtgtcagtcatgccatgcgtacgcaagcagggagaggctgaccgggagtggttcaacaccacaggtgcaggccttgcccgtgatgttgatcatgtggtgactactgctgaggttggtaagatattcctggagcgtggcatcaagctgaatgagctgccagagagcaactttgacaaccccattggcgagggcacaggtggtgctctgctgtttggcaccactggaggtgtcatggaggcagcacttcgcacagtctatgaagtggtgacccagaagcccatgggtcgtgttgactttgaggaggtgcgaggccttgaaggaatcaaggaggcagagatcacactcaagccaggagacgacagcccattcaaagccttcgcaggagctgatgggcagggcatcacgctcaagattgcagtagccaatgggcttggcaatgccaagaagctcatcaagagcctgtcagagggcaaggccaagtatgatttcattgaggtcatggcatgccctggtggctgcattggcggaggcggtcagccccgcagtactgacaagcagatcctgcagaagcgccagcaggctatgtacaacctggatgagcgcagtaccatccgccgcagccatgataacccattcatccaggcgctgtatgacaagttcctaggcgcacccaacagccacaaggcacatgatctgctgcacacacactatgtggcaggtggaattccagaggagaagtga

SEQ ID NO: 179 - DNA, 717 bp, Artificial sequence
  Green Fluorescent Protein
atggccaagggcgaggagctgttcaccggtgtggtccccatcctggtggagctggacggcgacgtgaacggccacaagttctccgtctccggcgagggtgagggtgacgccacctacggcaagctgaccctgaagttcatctgcaccaccggcaagctgcccgtgccctggcccaccctggtcaccaccctgacctacggtgtgcagtgcttctcccgctaccccgaccacatgaagcagcacgacttcttcaagtccgccatgcccgagggctacgtgcaggagcgcaccatcttcttcaaggacgacggcaactacaagacccgcgccgaggtcaagttcgagggcgacaccctggtgaaccgcatcgagctgaagggcatcgacttcaaggaggacggcaacatcctgggccacaagctggagtacaactacaactcccacaacgtgtacatcatggccgacaagcagaagaacggcatcaaggtgaacttcaagatccgccacaacatcgaggacggctccgtgcagctggccgaccactaccagcagaacacccccatcggcgatggccccgtgctgctgcccgacaaccactacctgtccatccagtccgccctgtccaaggaccccaacgagaagcgcgaccacatggtcctgctggagttcgtcaccgctgccggcatcacccacggcatggacgagctgtacaagtaa

SEQ ID NO: 180 - DNA, 320 bp, Artificial sequence
  Synthetic sequence
atccgtagttatccttatggccatcttagcgcagttgggtcaggggctggcgacgcgctgctgacgcgcaagtgaatggcccaacaagtcgcctcgcggtcgctgtcggcgccaaacccgcagctgcatccaccagattcacttgttagatcgacctaggttgcgggaccggaggcggctcgctgtgcaagcgcggtgacctcgtacggcggcatggatcgccatctcgattcgcgcggcagaatcgggccccgcgcacatttaagccgcgggcgagactcatttcgttacgtgcatcgattaacagcttctggacctga

SEQ ID NO: 181 - DNA, 580 bp, Artificial sequence
  Synthetic sequence
ttaaacgtcgtacgtccaagtataactaagccgacgtcgacccactctagaggatcgatccccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttggcagttgggtcaggggctggcgacgcgctgctgacgcgcaagtgaatggcccaacaagtcgcctcgcggtcgctgtcggcgccaaacccgcagctgcatccaccagattcacttgttagatcgacctaggttgcgggaccggaggcggctcgctgtgcaagcgcggtgacctcgtacggcggcatggatcgccatctcgattcgcgcggcagaatcgggccccgcgcacatttaagccgcgggcgagactcatttcgttaaatctgatacatgctattcagatcttacaa

SEQ ID NO: 182 - DNA, 580 bp, Artificial sequence
  Synthetic sequence
tcttccatcgtaaatctagcatcgattagcccgacgtcgacccactctagaggatcgatccccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttggcagttgggtcaggggctggcgacgcgctgctgacgcgcaagtgaatggcccaacaagtcgcctcgcggtcgctgtcggcgccaaacccgcagctgcatccaccagattcacttgttagatcgacctaggttgcgggaccggaggcggctcgctgtgcaagcgcggtgacctcgtacggcggcatggatcgccatctcgattcgcgcggcagaatcgggccccgcgcacatttaagccgcgggcgagactcatttcgttaatctgtaataatctagtcgaggcattcaag

SEQ ID NO: 183 - DNA, 777 bp, Artificial sequence
  Synthetic sequence
atctgtaataatctagtcgaggcattcaagatggccaagggcgaggagctgttcaccggtgtggtccccatcctggtggagctggacggcgacgtgaacggccacaagttctccgtctccggcgagggtgagggtgacgccacctacggcaagctgaccctgaagttcatctgcaccaccggcaagctgcccgtgccctggcccaccctggtcaccaccctgacctacggtgtgcagtgcttctcccgctaccccgaccacatgaagcagcacgacttcttcaagtccgccatgcccgagggctacgtgcaggagcgcaccatcttcttcaaggacgacggcaactacaagacccgcgccgaggtcaagttcgagggcgacaccctggtgaaccgcatcgagctgaagggcatcgacttcaaggaggacggcaacatcctgggccacaagctggagtacaactacaactcccacaacgtgtacatcatggccgacaagcagaagaacggcatcaaggtgaacttcaagatccgccacaacatcgaggacggctccgtgcagctggccgaccactaccagcagaacacccccatcggcgatggccccgtgctgctgcccgacaaccactacctgtccatccagtccgccctgtccaaggaccccaacgagaagcgcgaccacatggtcctgctggagttcgtcaccgctgccggcatcacccacggcatggacgagctgtacaagtaaaactggcttaaatcgttaacaatcgtgtga

SEQ ID NO: 184 - DNA, 320 bp, Artificial sequence
  Synthetic sequence
aactggcttaaatcgttaacaatcgtgtgaccgacgtcgacccactctagaggatcgatccccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttggatttaacataactgtcgattaccgtgcga

(19) United States
(12) Patent Application Publication
(10) Pub. No.: US 2005/0266541 A1
(43) Pub. Date: Dec. 1, 2005

(54) Methods and compositions for evolving microbial hydrogen production

(75) Inventor: Harrison F. Dillon, Palo Alto, CA (US)

Correspondence Address: SOLAZYME, INC., 3475 - T Edison Way, Menlo Park, CA, 94025, US

(73) Assignee: Harrison F. Dillon, Palo Alto, CA (US)

(21) Appl. No.: 10/763,712

(22) Filed: Jan. 21, 2004

Related U.S. Application Data
(63) Continuation-in-part of application No. 10/287,750, filed on Nov. 4, 2002, now abandoned.
(63) Continuation-in-part of application No. 10/411,910, filed on Apr. 12, 2003.
(63) Continuation-in-part of application No. 60/500,032, filed on Sep. 3, 2003.

(51) Int. Cl. C12Q 1/68; C12P 3/00; C12N 15/74; C12N 1/21

(52) U.S. Cl. 435/168; 435/252.3; 435/471

(57) ABSTRACT

The invention provides methods and compositions for engineering cells to generate large amounts of hydrogen. Genes that are involved in hydrogen production pathways and genes that are upregulated when cells are exposed to conditions conducive to the generation of hydrogen are mutagenized according to disclosed protocols. Microbes containing nucleic acid constructs are screened or selected for the ability to generate an increased amount of hydrogen. Methods of producing hydrogen are also disclosed.

This application claims priority to U.S. patent application Ser. No. 10/287,750, filed Nov. 4, 2002. This application also claims priority to U.S. patent application Ser. No. 10/411,910, filed Apr. 12, 2003. This application also claims priority to U.S. Patent Application No. 60/500,032, filed Sep. 3, 2003. U.S. patent application Ser. Nos. 10/287,750, 10/411,910, and 60/500,032 are hereby fully incorporated by reference for all purposes.

### BACKGROUND OF THE INVENTION

Hydrogen is the most abundant element on earth. When hydrogen is burned as a fuel, the only byproducts are heat and water. Large-scale commercial production of hydrogen could have a massive impact on the world environment and economy. The availability of an environmentally clean, renewable energy source would greatly curtail if not end large-scale dependence on fossil fuels. Hydrogen can be converted into electrical energy by utilizing fuel cells, but it would also be an ideal replacement for oil-based energy since it has a calorie per unit weight of 3 to 4 times that of petroleum (U.S. Pat. No. 4,532,210).

Fuel cell technology is being developed at a rapid pace, however a plentiful and commercially viable source of hydrogen with which to run fuel cells has not yet been created. There are a variety of known methods for producing hydrogen. For instance, inorganic membrane electrolysis technology (IMET) involves the splitting of water through electrolysis in the reaction 2H₂O=>2H₂+O₂. Water electrolysis occurs through passing an electric current through water to separate it into hydrogen and oxygen Hydrogen gas is produced at the negative cathode and oxygen gas is produced at the positive anode. Another source of hydrogen production is through reforming natural gas. Unfortunately this process produces carbon dioxide making this source of hydrogen less than ideal.

Hydrogen production through electrolysis, powered by renewable sources such as wind, solar energy through photovoltaic cells, or hydroelectric power has the advantage of not creating pollutants in the process of generating hydrogen, however the potential amount of hydrogen that can be produced through these methods may be limiting.

What is needed are methods for engineering microbial organisms to produce hydrogen for extended periods of time in large amounts, something no known microbe is currently capable of doing. Furthermore, methods of identifying genes that are involved in hydrogen production pathways of microbes so that they can be optimized for efficient contribution to the production of hydrogen are needed.

### BRIEF SUMMARY OF THE INVENTION

Provided are sethod for engineering a cell to produce an increased amount of hydrogen comprising providing a mutagenized nucleic acid sequence derived from a first gene that encodes a protein involved in a hydrogen production pathway, transforming a cell with the mutagenized nucleic acid sequence, and screening or selecting the cell for an increased amount of hydrogen.

Methods are provided for identifying a first independent transformant which produces an increased amount of hydrogen, recovering the mutagenized nucleic acid sequence from the independent transformant, further mutagenizing the recovered mutagenized nucleic acid sequence to create a new library of mutagenized nucleic acid sequences, transforming cells with the new library of mutagenized nucleic acid sequences, and screening or selecting for a new independent transformant that generates an increased amount of hydrogen compared to the first independent transformant.

In some methods a plurality of mutagenized nucleic acid sequences are recovered from a plurality of independent transformants which produce an increased amount of hydrogen, wherein the plurality of mutagenized nucleic acid sequences are subjected to gene reassembly to generate the new library.

In one embodiment a plurality of mutagenized nucleic acid sequences are used to transform a population of cells, followed by the screening or selecting.

In one embodiment the first gene is selected from the group that encodes ferredoxin, catalase, isoamylase, malate dehydrogenase, 14-3-3 protein, enolase, aldolase, ribosomal protein S8, ribosomal protein L17, ribosomal protein S18, ribosomal protein L37, ribosomal protein L12, ribosomal protein S15, iron-hydrogenase, nickel-iron hydrogenase, and components of the photosystem I, photosystem II, light harvesting antenna and cytochrome b₆-f complexes.

The methods provided include mutagenesis of iron hydrogenase proteins including mutagenesis of the X¹X²X³X⁴X⁵X⁶GGVMEAAX⁷R and ADX⁸TIX⁹EE segments. In some methods, cognate sequences of these conserved segments of iron hydrogenases are substituted into a Chlamydomonas iron hydrogenase. In some methods, gene reassembly methods are performed in which a Chlamydomonas iron hydrogenase is mutagenized by incorporation of segments of iron hydrogenase proteins from other species. Preferred segments for inclusion in gene reassembly include segments that form parts of the gas channel, also referred to as the gas channel. In some methods a higher molecular weight amino acis is substituted into a gas channel segment, such as a tryptophan for the methionine in the C. reinhardtii TIMEE segment. In other gene reassembly methods the iron hydrogenase is reassembled using methods that involve attaching sections of duplex DNA that have only one overhanging nucleotide. In other methods oligonucleotides encoding gas channel segments are annealed to a scaffold nucleic acid, where the oligonucleotides anneal to non-overlapping sites. Preferably, the mutagenesis of a hydrogenase does not decrease the protein's ability to accept electrons from an electron donor. In some methods the mutagenized nucleic acid is transcribed by a light-driven promoter.

Methods are provided herein for screening or selecting for a hydrogen production phenotype in the presence of oxygen at a concentration selected from the ranges comprising more than 0.5%, more than 5.0%, more than 10%, more than 15%, approximately 21%, more than 21%, more than 25%, more than 30% or more than 35% oxygen. In some methods the cells screened or selected are in liquid culture media.

Methods are provided for mating (a) at least one cell of a strain containing a mutagenized form of the first gene, wherein the at least one cell is identified by the screening or selecting or wherein the at least one cell is derived through mating from a cell identified by the screening or selecting; (b) to at least one cell of a distinct strain containing a mutagenized form of the second gene, wherein the at least one cell is identified by the screening or selecting, or wherein the at least one cell is derived through mating from a cell identified by the screening or selecting; and (c) screening or selecting for a progeny cell that produces an increased amount of hydrogen compared to any parent cell.

A method of hydrogen production is disclosed, comprising placing cell containing a mutagenized nucleic acid sequence corresponding to a gene that is involved in a hydrogen production pathway into liquid culture media or on to solid culture media, wherein the mutagenized nucleic acid sequence is operably linked to a transcriptional promoter sequence; culturing said transformed cell under conditions sufficient to stimulate transcription of said mutagenized nucleic acid sequence(s); and collecting an evolved gas. In some methods the culture media supplied to the cells is photoautotrophic growth requiring media

Mating methods are provided. One method is a method of multiparental mating of microbes that mate in response to a stimulus, comprising: (a) providing a cell from each of 3 or more strains of microbes capable of mating to each other in culture medium, (b) providing the stimulus; (c) allowing cells to mate and produce progeny; (d) allowing the progeny cells to achieve sexual reproduction capability; (e) providing the stimulus at least one more time; and (f) screening or selecting the further progeny for a desired phenotype. In some methods the microbes are green algae and the stimulus is the removal of nitrogen from the media and illumination by light comprising a wavelength of light between about 0.420.52 micrometers. In some methods the green algae are of the Chlamydomonas genus, optionally of a species selected from the group comprising reinhardtii, eugametos, incerta, and moewusii. In other methods the stimulus is interruption of exponential growth in continuous light with a reduction in light, followed by addition of light, wherein the reduction in light occurs for a period selected from the group consisting of at least 1, 2, 3, 4, 5, 6, 7, 8, 9, 10, 11, 12, or more than 12 hours. In other methods the microbes are of the Scendesmus genus and the stimulus is the addition of chromium to the culture media. In some methods the desired phenotype is hydrogen production. In still other methods, nucleic acid exchange occurs between only two parental cells at a time during the mating process.

The foregoing description of some preferred embodiments of the invention is not a limiting description of the invention, and many other embodiments of the invention are described herein.

### BRIEF DESCRIPTION OF THE DRAWINGS

FIG. 1 demonstrates the method of subjecting homologous genes cloned from different microbes capable of producing hydrogen to Dnase I digestion in preparation for DNA shuffling procedures.

FIG. 2 demonstrates the construction of a library of shuffled sequences. Dnase I digested fragments are annealed to chimeric oligonucleotides that contain sequences corresponding to the N and C terminal ends of the coding regions of the shuffled genes as well as linker sequences referred to as “unique sequences” that are present at both ends of each fragment after annealing and primerless PCR

FIG. 3 demonstrates the denaturation, annealing, and primerless PCR of DNA fragments containing different elements of a DNA construct used to transform cells. Denatured fragments anneal through unique sequences to other fragments. The shuffled library of coding regions of shuffled differentially regulated genes is flanked by unique sequences that anneal to promoter and transcriptional terminator sequences.

FIG. 4 depicts a map of the DNA constructs described in Example 1, with details demonstrating the annealing points of each shuffled library to flanking nonshuffled segments during construction.

FIG. 5 depicts a map of the DNA constructs described in Example 1.

FIG. 6 depicts a detailed map of the DNA constructs described in Example 1, including the relative positions of PCR primers and chimeric oligonucleotides. The map is not necessarily drawn to scale.

FIG. 7 depicts a detailed map of the DNA constructs described in Example 2, including the relative positions of PCR primers and chimeric oligonucleotides. The map is not necessarily drawn to scale.

FIG. 8 depicts a screening system for use with liquid culture-containing multiwell plates.

FIG. 9 depicts amino acid residues in and near the gas channel of the Clostridium pasteurianum iron hydrogenase from the structure 1feh in the Protein Data Bank The amino acid positions from the Clostridium pasteurianum iron hydrogenase are shown in italics, while the corresponding amino acid positions from a Chlamydomonas reinhardtii iron hydrogenase are shown above in non-italicized font, both according to the numbering from FIG. 4 of Happe, Eur J Biochem (2002) February; 269(3): 1022-32.

FIG. 10 depicts the codon usage table of C. reinhardtii. Most preferred codons are shown underlined and in bold-face type. Any cDNA sequence can be recoded for maximal expression in C. reinhardtii by substituting non-preffered codons for most preferred codons. Codon usage tables for microbes can be found at http://www.kazusa.or.jp/codon/.

FIG. 11 depicts the mating of two C. reinhardtii cells. Genetic alterations on cognate chromosomes that each increase hydrogen production can cosegregate in a progeny cell through a recombination event. Such progeny can produce more hydrogen than parental strains.

FIG. 12 depicts multiparental mating of four strains of C. reinhardtii. Each of the four strains has a genetic alteration that increases hydrogen production. The multiparental mating reaction proceeds through at least two cycles of nitrogen deprivation and germination. All four genetic alterations can cosegregate in a progeny cell. Such progeny can produce more hydrogen than either parent strain in any of the matings that occur in the multiparental mating reaction.

FIGS. 13-14 depict a gene reassembly protocol for incorporating segments of diverse Iron hydrogenaserogenases into the overall framework of a single Iron hydrogenaserogenase. In this example, a C. reinhardtii Iron hydrogenaserogenase gene provides the single stranded framework. The design of the protocol allows framework/hinge regions to be retained while architecture of the gas channel is altered compared to the C. reinhardtii Iron hydrogenaserogenase.

FIG. 15 shows the key to the identity of the amino acids of step 1 of FIG. 13 and the corresponding identity of codons in nucleic acids in steps 2-9 of FIGS. 13-14.

FIG. 16 shows the divergent sequences from SEQ ID Nos: 1-112 that correspond to the segments of Iron hydrogenaserogenases that line the gas channel. These are the segments that are schematically depicted in FIG. 13, step 1. The sequences are used to design the oligonucleotides in step 2 of FIG. 13.

FIG. 17 shows one example of how gas channel segments from SEQ ID Nos: 1-112 are reverse translated into recoded nucleotide sequence. C. reinhardtii flanking sequence is added to each side of the oligonucleotide sequence to ensure adequate annealing. Although step 1 of FIG. 13 depicts 3 segments, which FIG. 16 shows only 2 segments, the X¹X²X³X⁴X⁵X⁶GGVMEAAX⁷R segment is broken into two distinct segments to allow greater combinatorial diversity af the library, as this figure shows.

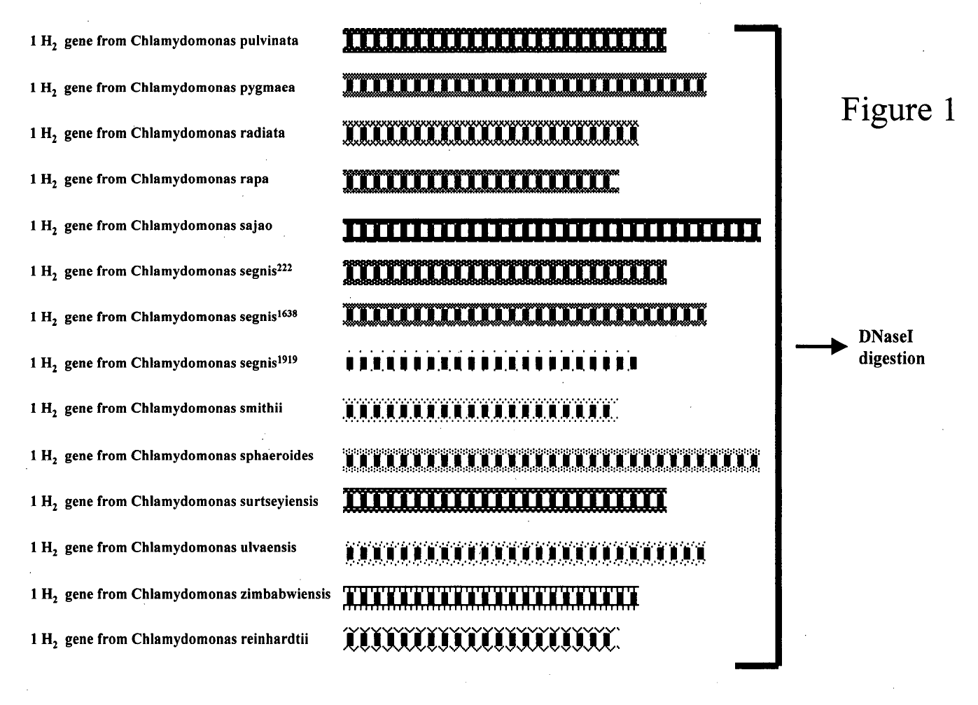

### DETAILED DESCRIPTION OF THE INVENTION

All publications, patents, patent applications, and other references cited are fully incorporated by reference for all purposes.

Definitions: The following definitions are intended to convey the intended meaning of terms used throughout the specification and claims, however they are not limiting in the sense that minor or trivial differences fall within their scope.

“Differential expression profile” means information about the activity of at least one gene or the presence or activity of at least one protein in a cell when the cell is exposed to at least two different environmental conditions or chemical environments. Literally any difference in the conditions that the cell might be exposed to can cause a difference in the expression of one or more genes or the presence or activity of one or more proteins.

“Conditions more conducive to the generation of hydrogen” means any set of conditions under which a cell generates hydrogen.

“Conditions more conducive to the generation of hydrogen” also means, in an experiment intended to generate a differential expression profile, conditions under which a cell that already generates a measurable amount of hydrogen under a first set of conditions generates, under a second set of conditions distinct from the first set, a measurably greater amount of hydrogen than it does under the first set of conditions.

“Conditions less conducive to the generation of hydrogen” means any set of conditions under which a cell either generates no measurable amount of hydrogen or generates measurably less hydrogen than under conditions more conducive to the generation of hydrogen. Specifically, conditions more conducive to the generation of hydrogen cause a cell to generate a measurable amount of hydrogen while conditions less conducive to the generation of hydrogen cause a cell to generate either no hydrogen or measurably less hydrogen than the conditions more conducive to the generation of hydrogen in that same experiment. When cells are cultured under conditions less conducive to the generation of hydrogen yet produce a measurable amount of hydrogen, that measurable amount of hydrogen is less than the amount of hydrogen produced by cells cultured under conditions more conducive to the generation of hydrogen in order to produce a differential expression profile. In terms of measuring the amount of hydrogen produced, a greater amount of hydrogen produced by a cell under one condition compared to another condition is determined by measuring production of hydrogen over a given time interval.

“Conditions not conducive to the generation of hydrogen” means any set of conditions under which a cell does not generate a measurable amount of hydrogen.

“Culture conditions” and “conditions” means the plurality of variables that are manipulated when culturing microbes, including but not limited to exposure to light or certain wavelengths of light, exposure to certain molecules, nutrients, elements, and the like in culture media as well as exposure to different concentrations of these molecules, elements, nutrients, and the like, temperature, placement in darkness or partial darkness, exposure to other microbes or viruses, as well as any other variable that is manipulated when culturing microbes.

“Differentially regulated” means where the activity of a gene or a protein in a cell is in some way different under one set of culture conditions than under a different set of culture conditions. For instance, Chlamydomonas cells express certain genes in higher amounts during the first hour of anaerobic culturing in the dark as compared to culturing in the presence of oxygen and illumination. Even though certain genes are expressed in both culture conditions, if the genes are expressed at different levels between the two conditions they are differentially regulated.

“Mutagenized nucleic acid sequence” means a nucleic acid sequence in which the nucleotide sequence of the mutagenized nucleic acid sequence differs from a starting sequence prior to mutagenesis by at least one base pair. For instance, a single nucleic acid sequence is amplified using error-prone PCR to generate a library of nucleic acid sequences that are similar in sequence to the starting sequence but differ by at least one base pair, and are therefore mutagenized nucleic acid sequences. Alternatively, a plurality of nucleic acid sequences that have significant sequence identity are put through a gene reassembly process to generate mutagenized nucleic acid sequences. Mutagenized nucleic acid sequences are derived from the fill or partial sequence of at least one wild type sequence, also referred to as a starting sequence. In gene reassembly processes the starting sequences are the parental genes in non-recombined form. Mutagenized nucleic acid sequences can also be generated by chemical mutagenesis of living cells using carcinogens such as nitrosoguanidine (NTG).

“Significant sequence identity” means at least 40%, preferably 50%, more preferably 60% and more preferably 70%, and even more preferably 80% or 90% or higher nucleotide sequence identity when compared using a standard sequence comparison such as the BLAST program available at www.ncbi.nlm.nih.gov. utagenized nucleic acid sequences can also be generated using standard site-directed mutagenesis protocols (Maniatis et al. (1989) Molecular Cloning: A Laboratory Manual Cold Spring Harbor Laboratory).

“Downregulated” means, when relating to a gene, when a gene is transcribed less per unit time or when a gene's corresponding RNA is translated less times per unit time than it was when compared to the level of transcription or translation previously. “Downregulated” means, when relating to a protein, when the protein's activity per unit time is diminished when compared to the level of activity per unit time previously, when the protein is degraded at a faster rate, or when the gene encoding the protein is transcribed less per unit time or is translated less times per unit time than it was when compared to the level of transcription or translation previously.

“Upregulated” means, when relating to a gene, when a gene is transcribed or when a gene's corresponding RNA is translated more times per unit time than it was when compared to the level of transcription or translation previously. “Upregulated” means, when relating to a protein, when the protein's activity per unit time is increased when compared to the level of activity per unit time previously, when a protein is degraded at a slower rate, or when the gene encoding the protein is transcribed more per unit time or is translated more times per unit time than it was when compared to the level of transcription or translation previously.

“Shuffling” means recombining a first nucleic acid with at least one other nucleic acid distinct in sequence from the first nucleic acid, wherein the first nucleic acid and the at least one other nucleic acid recombine through sequence-specific annealing with each other or to a third nucleic acid. Shuffling is also referred to as gene reassembly.

“Site-directed mutagenesis” means generating a desired gene sequence that differs from the sequence of a starting gene, wherein the sequence difference is a specifically designed amino acid insertion, deletion, substitution, or combination thereof.

“Increased amount of hydrogen” means an amount of hydrogen produced by a strain that has been transformed with a mutagenized nucleic acid sequence that is greater than the amount of hydrogen produced by the starting strain that has either not been transformed with the mutagenized nucleic acid sequence or that has been transformed using only control or vector sequences.

A cell “derived through mating” from a distinct cell is a cell that would not exist but for the mating of the distinct cell with at least one other cell. For example, a distinct cell has a mutagenized nucleic acid sequence that causes increased hydrogen production. The distinct cell is mated to another cell, resulting in progeny cells. The progeny cells are derived through mating from the first cell.

### DESCRIPTION

Culturing Bacteria Under Conditions More Conducive to the Generation of Hydrogen

Methods for culturing photosynthetic bacteria under conditions more conducive and less conducive to the generation of hydrogen are known (Maness, (2001) Appl Microbiol Biotechnol December; 57(5-6):751-6; Weaver P F, Proceedings of the Fifth Joint US/USSR Conference of the Microbial Enzyme Reactions Project, Jurmala, Latvia, USSR (1979) 461-479). Methods for culturing cyanobacteria under conditions more conducive and less conducive to the generation of hydrogen are known (Masukawa, Appl Microbiol Biotechnol 2002 April; 58(5):618-24; Benneman J R. Proceedings of the 10th World Hydrogen Energy Conference, Cocoa Beach, Fla., USA (1994); Papen, Biochimie 1986 January; 68(1):121-32). Methods for culturing other bacteria such as E. coli under conditions more conducive and less conducive to the generation of hydrogen are known (Nandi, J Bacteriol 1985 April; 162(1):353-60). The culture media may be solid or liquid.

Standard growth media for other types of cells such as bacteria, cyanobacteria, and photosynthetic bacteria are known (see Maniatis et al. (1989) Molecular Cloning: A Laboratory Manual Cold Spring Harbor Laboratory; Masukawa, Appl Microbiol Biotechnol 2002 April; 58(5):618-24; and Papen et al., Biochimie 1986 January; 68(1):121-32; Dzelzkalns, J Bacteriol 1986 March; 165(3):964-71). Preferably the cells are cultured in liquid media during a screening or selection process since a desired strain that is capable of generating large amounts of hydrogen in the presence of oxygen is commercially deployed in liquid media

Culturing Green Algae Under Conditions Less Conducive to the Generation of Hydrogen

Green algae such as Chlamydomonas reinhardtii are grown in atmospheric conditions (ie: normal air), with or without illumination, according to standard protocols (Harris, (1989) The Chlamydomonas Sourcebook. Academic Press, New York; Rochaix J-D et al. (1998) The Molecular Biology of Chloroplasts and Mitochondria in Chlamydomonas (Advances in Photosynthesis, Vol 7). A culture is grown for any period of time under these conditions. Although it is desired to grow the cells overnight to obtain a healthy culture, if the starting cells were also grown under any conditions less conducive to the generation of hydrogen the culture need not be grown for a long periods of time. All that is necessary is for the cells to be cultured for some amount of time, preferably at least 5 minutes under conditions less conducive to the generation of hydrogen, before harvesting. More preferably, the cells are cultured for one or more hours before harvesting. Alternatively, cells are grown and then frozen. The exact conditions and duration of culturing are not vitally important, and trivial differences can be incorporated into the protocol, as long as the cells were not placed in conditions more conducive to the generation of hydrogen within at least about 10 minutes before harvesting. For example, the cells are cultured in Sager's minimal media or TAP media in light.

Culturing Green Algae Under Conditions More Conducive to the Generation of Hydrogen

In one example, green algae such as C. reinhardtii are cultured under conditions in which no sulfur is present in the media and atmospheric oxygen is not present in any gas space contacting the media After about 15 hours under such conditions, green algae cells begin producing hydrogen. (Zhang, Planta (2002) February; 214(4):552-61; Melis, Plant Physiol (2000) January; 122(1):127-36). In other methods, cells are provided minimal amounts of sulfur, such as between 10 and 50 micromolar sulfur, and under such conditions cells generate hydrogen (Kosourov, Biotechnol Bioeng 2002 Jun. 30; 78(7):73140).

Preferably the cells are cultured in liquid media during a screening or selection process since a desired strain that is capable of generating large amounts of hydrogen in the presence of oxygen is commercially deployed in liquid media. In other words, it is desirable to screen or select for cells in the same type of media as will be used for commercial hydrogen production. For this reason liquid growth media is preferred. Growth media for Chlamydomonas cells, such as Sager's Minimal Media and Hunters Trace Element Media, are described in sources such as Harris E., (1989) The Chlamydomonas Sourcebook. Academic Press, New York and Rochaix J-D et al. (1998) The Molecular Biology of Chloroplasts and Mitochondria in Chlamydomonas (Advances in Photosynthesis, Vol 7). These growth media can be made as solid agar or as liquid. Other green algae media can be used, such as Tris-Acetate-Phosphate (TAP) media or Sueoka's media, as described in Harris and other sources. Minimal media such as Sager's (also known as Sager-Granick) is preferred when the host organism is or can be photoautotrophic because it is desirable to evolve microbes to generate hydrogen using only sunlight as energy. Sager's media is an example of photoautotrophic growth requiring media

Any component of the culture media may be manipulated. For example, a selection molecule such as an antibiotic is added to the culture media and a corresponding selectable marker gene is incorporated into the transformation vector containing the recoded and recombined hydrogenase library.

Optionally, other components of the culture media are manipulated such as amount of sulfur in the media. The level of sulfur may be increased, decreased, or held constant throughout the period of culture. (see Melis et. al. Plant Physiol (2000) January; 122(1):127-36 and Zhang et al. Planta (2002) February; 214(4):552-61).

Another component that may be optionally added to the culture media is metronidazole (MNZ). MNZ is a strong oxidizer of reduced ferredoxin. Ferredoxin accepts electrons from the Photosystem I complex and transfers them to the hydrogenase to supply electrons for the 2H⁺+2^{e-}→H₂ reaction. When MNZ is added to the culture media a controlled amount of oxygen is also added to the culture container and cells that survive are assayed for hydrogen production. In a typical experiment, C. reinhardtii cells that survive the MNZ treatment protocol, cultured for example in Saeger's minimal media in 20 mM MNZ; 1 mM Sodium Azide; 2% oxygen, 200 W/m² light for 20 minutes, with expression of one or more mutagenized nucleic acid sequences, are placed in liquid culture media in multiwell plates and assayed for hydrogen production. It is unnecessary to count the number of independent transformants that survive the MNZ treatment. Any transformant that survives the treatment is capable of producing more hydrogen under a certain level of oxygen than a wild-type cell, and therefore all survivors are assayed for hydrogen production without regard to the number or percent of mutant survivors. For an example of the use of MNZ, see U.S. Pat. No. 5,871,952.

In one embodiment, cells are cultured in a Tris-acetate-phosphate media, at approximately pH 7.0 (Harris, (1989) The Chlamydomonas Sourcebook. Academic Press, New York). The cultures are bubbled with 3% CO₂ in air at 25° C. The cultures are continuously illuminated. After at least five minutes of culturing under these conditions, cells are harvested and are resuspended in the same media as before except for the absence of sulfur. The cells are then cultured under continuous illumination Alternatively, the cells are originally cultured in the absence of acetate, but under continuous illumination (ie: photoautotrophically), and are then transferred to media that contains an absence of sulfur. Alternatively, culture conditions comprise culturing the cells in media that is devoid of sulfur, iron, or manganese, or any combination of these three elements.

In another embodiment, frozen aliquots of green algae are thawed in culture media devoid of sulfur and continuously cultured, in the presence of light, for at least five minutes. The cells are then harvested.

There are other culture conditions for some algae species that are conducive to the generation of hydrogen besides the sulfur deprivation method. For instance, blue-green algae produce hydrogen when starved of nitrogen (Weissman, Appl Environ Microbiol 1971 January; 33(1):123-31). Hydrogen is also generated when green algae are cultured in the absence of light when the culture is flushed with gases, such as argon, that remove oxygen from the media (Happe, Eur J Biochem (2002) February; 269(3): 1022-32).

Generation of a Differential Expression Profile: Comparison of RNA Between Cells Cultured in Conditions More Conducive to the Generation of Hydrogen and Cells Cultured in Conditions Less Conducive to the Generation of Hydrogen

Once at least two sets of cells are cultured under conditions more conducive and less conducive to the generation of hydrogen, RNA samples are extracted from the cells. Methods and protocols for the isolation of RNA from bacterial and algae cells are well known in the art (Maniatis et al. (1989) Molecular Cloning: A Laboratory Manual Cold Spring Harbor Laboratory; Harris, (1989) The Chlamydomonas Sourcebook. Academic Press, New York; Rochaix J-D et al. (1998) The Molecular Biology of Chloroplasts and Mitochondria in Chlamydomonas (Advances in Photosynthesis, Vol 7).

The RNA is isolated from both the cells placed under conditions more conducive to the generation of hydrogen as well as cells placed under conditions less conducive to the generation of hydrogen. There is no requirement that both sets of cells be grown simultaneously or that RNA be isolated from both sets of cells simultaneously. There is also no requirement that the same strain of microbe be used in both culture conditions, although it is preferred that they be the same strain.

After RNA is isolated from the cells, a plurality of methods can be utilized to generate a differential expression profile.

In one embodiment, the RNA is placed on microarrays such as silicon chips or glass slides containing sequences corresponding to known sequences from the genome of the cells. It is not necessary that the sequences immobilized onto the microarray are derived from the same strain or species of the cells from which RNA are isolated as long as the genome of the cells used to make the microarray is somewhat homologous to the genome of the cells from which the RNA is isolated. For instance, the cells exposed to conditions more conducive and less conducive to the generation of hydrogen are Chlamydomonas fusca while the sequences immobilized on the microarrays are Chlamydomonas reinhardtii. Utilizing evolutionarily related strains of microbes for purposes of RNA isolation and microarray sequence immobilization provides reliable data, and the methods disclosed herein are utilized with a variety of microbes. RNA molecules isolated from cells hybridize with nucleic acid molecules immobilized on the microarray to form double stranded RNA duplexes. Such duplexes are detected by a variety of methods known in the art (such as the GeneChipe product and associated scanning techniques produced by Affymetrix Inc., Santa Clara, Calif.; Dudley, Proc Natl Acad Sci USA 2002 May 28; 99(11):7554-9). In one embodiment the RNA isolated from cells is amplified by PCR and labeled nucleotides are incorporated into the newly synthesized nucleic acid molecules. These molecules are digested with a nuclease, denatured to single stranded molecules, and hybridized to the immobilized sequences on the chip. Double stranded duplexes that form contain the labeled nucleotides from the PCR reaction in one strand, and these duplexes are visualized. For example, the label incorporated into the molecules in the PCR reaction is a fluorescent molecule, and the microarray is placed into a fluorescence detection chamber. Such microarray technology is well known in the art. For instance, microarrays containing over 2,700 unique genes from C. reinhardtii are commercially available (Chlamydomonas Genome Project, Duke University, Durham, N.C.). In addition to the ability to visualize whether or not a duplex has formed on a particular spot corresponding to a particular gene on the chip, this technology also quantitates the difference in the amount of duplex formed on a given spot between two or more experiments using different RNA samples. This differentiation ability allows the identification of differentially regulated genes between cells grown in culture conditions more conducive to the generation of hydrogen and less conducive to the generation of hydrogen.

Upon hybridization of the RNA samples from two or more sets of cells, genes that are upregulated or downregulated between the two sets of cells are identified. For example, the iron hydrogenase gene in Chlamydomonas is turned on when the cells are exposed to conditions more conducive to the generation of hydrogen, however the gene is turned off when the cells are exposed to conditions not conducive to the generation of hydrogen When the two RNA samples are placed on microarrays containing immobilized sequences corresponding to the genome of C. reinhardtii, a spot on the chip containing the sequence of the iron hydrogenase gene contains a duplex of nucleic acid when the RNA sample is isolated from cells exposed to conditions more conducive to the generation of hydrogen, whereas the spot does not contain a duplex when the RNA sample is isolated from the cells exposed to conditions not conducive to the generation of hydrogen. The C. reinhardtii iron hydrogenase gene is differentially regulated between cells exposed or not exposed to conditions more conducive to the generation of hydrogen, and therefore the gene is identified as differentially regulated.

Generation of a Differential Expression Profile: Suppression Subtractive Hybridization Between Cells Cultured in Conditions More Conducive to the Generation of Hydrogen and Cells Cultured in Conditions Less Conducive to the Generation of Hydrogen

In another embodiment, RNA is isolated from both sets of cells and is put through the Suppression Subtractive Hybridization PCR technique (Diatchenko, Proc Natl Acad Sci U S A 1996 Jun. 11; 93(12):6025-30; Happe, Eur J Biochem (2002) February; 269(3):1022-32; commercially available kits are provided by Clontech Laboratories, Inc., Palo Alto, Calif.). In this technique transcripts from genes expressed in one sample (in this case the cells cultured under conditions more conducive to the generation of hydrogen) but not the other (in this case the cells cultured under conditions less or not conducive to the generation of hydrogen) are selectively amplified through the PCR method. Genes amplified through this technique are differentially regulated genes.

Generation of a Differential Expression Profile: Two Dimensional Gel Electrophoresis Between Cells Cultured in Conditions More Conducive to the Generation of Hydrogen and Cells Cultured in Conditions Less Conducive to the Generation of Hydrogen

A differential expression profile is created by subjecting protein samples from both sets of cells to two dimensional gel electrophoresis. This technique is well known in the art, and is optionally coupled with mass spectrometry techniques to aid in the identification of proteins (Arthur, Kidney Int 2002 October; 62(4):1314-21). Spots indicating proteins on a gel from cells exposed to conditions more conducive to the generation of hydrogen but not present or present in different amounts on a gel from cells exposed to conditions less conducive to the generation of hydrogen correspond to proteins encoded by differentially regulated genes. Two dimensional gel electrophoresis analysis is advantageous for purposes such as monitoring the content of organelles such as chloroplast or multiprotein complexes such asphotosystem I that are involved in the production of hydrogen. (Dreger, Eur J. Biochem. 2003 February; 270(4):589-99).

Generation of a Differential Expression Profile: Other Methods:

In another embodiment, a differential expression profile is created by analyzing only a single gene or a small set of genes through methods such as Northern blotting, Western blotting, or activity assays specific to a protein of interest (Maniatis et al. (1989) Molecular Cloning: A Laboratory Manual Cold Spring Harbor Laboratory). A plurality of methods, specific to each gene, is employed to assess a difference in the activity of a gene or protein between two or more samples of cells exposed to different conditions. Any difference in conditions that a cell is exposed to may cause differential activity of some genes and/or proteins, including but not limited to components of culture media, temperature, exposure to sunlight or light of varying wavelengths, the presence of specific nutrients or elements, exposure to certain molecules, and exposure to other organisms or viruses.

Identification of Differentially Regulated Genes

After generation of the differential expression profile, any gene or protein demonstrated to be differentially regulated when cells are exposed to conditions more conducive to the generation of hydrogen versus conditions less conducive to the generation of hydrogen is a target for engineering efforts. For instance, the iron hydrogenase gene in C. reinhardtii is differentially regulated between conditions more conducive to the generation of hydrogen and conditions less conducive to the generation of hydrogen.

Also provided are methods for the identification of genes and proteins down-regulated when cells are exposed to conditions more conducive to the generation of hydrogen. Such genes are targets for mutation, deletion from the genome, or downregulation through methods such as RNA interference. Alternatively, molecules capable of inhibiting the activity of proteins downregulated when cells are exposed to conditions more conducive to the generation of hydrogen are added to the culture in order to stimulate the cells to generate an increased amount of hydrogen.

Providing Mutagenized Nucleic Acid Sequences Corresponding to Differentially Regulated Genes

Clones of genes identified as differentially regulated are obtained. Creation of full-length cDNA molecules is standard in the art (Maniatis et al. (1989) Molecular Cloning: A Laboratory Manual Cold Spring Harbor Laboratory), however gene fragments are also used. The gene or gene fragment is mutagenized using one or more mutagenesis methods.

In one embodiment, the gene is amplified using error-prone PCR-Error-prone PCR is a standard procedure in the art (Leung, Technique (1989) 1, 11-15). In this technique the gene of interest is amplified using a DNA polymerase under conditions that are deficient in the fidelity of replication of sequence. The result is that the amplification products contain at least one error in the sequence. When a gene is amplified and the resulting product(s) of the reaction contain one or more alterations in sequence when compared to the template molecule, the resulting products are mutagenized as compared to the template.

Alternatively, the gene of interest is cloned into a suitable vector and used to transform a microbe. The microbe is then grown while exposed to a mutagenizing agent such as nitrosoguanidine or ethyl methanesulfonate (Nestmann, Mutat Res 1975 June; 28(3):323-30), and the vector containing the gene is then isolated from the host.

In one embodiment, the gene identified as upregulated is mutagenized through gene reassembly, saturation mutagenesis, or other directed evolution techniques. These techniques are known in the art (U.S. Pat. No. 5,605,793, U.S. Pat. No. 5,830,721, U.S. Pat. No. 6,165,793, U.S. Pat. No. 6,180,406, U.S. Pat. No. 5,939,250, U.S. Pat. No. 6,171,820, U.S. Pat. No. 6,361,974, U.S. Pat. No. 6,358,709, U.S. Pat. No. 6,352,842, U.S. Pat. No. 6,238,884, U.S. Pat. No. 6,420,175, U.S. Pat. No. 6,287,861 and related patents; Coco et al., Nat Biotechnol 2001 April; 19(4):354-9).

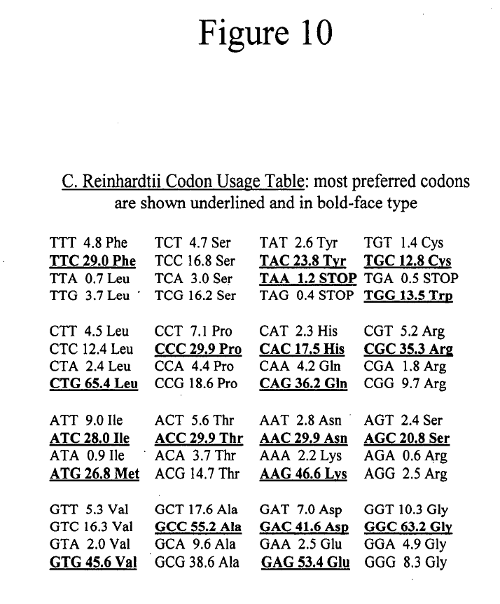

It is preferable but not necessary that nucleic acid molecules used in shuffling protocols use the same codon to encode each individual amino acid. For example, even though 6 different amino acids encode Arginine, only CGC is used. It is also preferable that the codon used to encode each amino acid is the most preferred codon in an organism that is transformed with the shuffled sequences. Using only one codon that is the most preferred codon in the organism is preferred because it allows the nucleic acid fragments to anneal better because they have higher nucleotide sequence identity. In addition, every protein encoded by a shuffled sequence is translated at equal efficiency by the organism. In one embodiment, the organism is C. reinhardtii, at least nucleic acid molecule encoding one segment of a protein from SEQ ID NOs: 1-112 is used in a shuffling protocol, and the nucleic acid molecules that are used in the shuffling protocol use only the most preferred codon from C. reinhardtii as depicted in FIG. 10.

In one embodiment, the differentially regulated gene is digested with a nuclease such as Dnase I to form random fragments. These fragments are mixed with similarly digested fragments of at least one other gene that contains some sequence homology to the differentially regulated gene. Alternatively the fragments are pooled with synthetic single or double stranded oligonucleotides corresponding to sequences from genes possessing homology or partial homology to the differentially regulated gene. The mixed fragments are denatured to form single stranded molecules and the molecules are then allowed to anneal to each other. The fragments are put through an extension protocol such as primerless PCR in which 3′ ends of fragments are extended through the use of a DNA polymerase enzyme. The resulting mixture contains a library of shuffled sequences that are used to transform cells for screening or selection procedures.

In one embodiment genes that are homologous to genes that are (a) identified as differentially regulated and (b) are further identified as upregulated when cells are exposed to conditions more conducive to the generation of hydrogen are isolated from evolutionarily similar microbes. For example, the iron hydrogenase gene is upregulated in C. reinhardtii when the cells are exposed to conditions more conducive to the generation of hydrogen. Other iron hydrogenase genes are isolated from microbes that are evolutionarily related and/or are known to possess an iron hydrogenase gene. For sequences of genes homologous to the gene identified as differentially regulated that are already known, gene fragments corresponding to these genes may be chemically synthesized using known sequence information; it is not necessary that such genes be actually cloned from their natural source in order to be utilized in shuffling experiments. Examples of such known iron hydrogenase genes include those listed in the sequence listing.

In one embodiment, nucleic acid fragment encoding proteins sequences of at least 5 amino acids are used in shuffling experiments. Alternatively, the fragments encode at least 6 amino acids, and in some instances at least 8 9, 10, 11, 12, 13, 14, 15, 16, 17, 18, 19, 20, 21, 22, 23, 24, or 25 or more amino acids.

These genes are isolated through procedures known in the art. For instance, the C. reinhardtii iron hydrogenase gene is used as a probe to screen cDNA or genomic DNA libraries of other green algae. In particular, the highly conserved “H-cluster” sequence corresponding to the active site of iron hydrogenases is used as a probe (Peters, Science (1998) December 4;282(5395):1853-8, Nicolet, Structure Fold Des (1999) January 15; 7(1):13-23). Alternatively, PCR primers corresponding to sequences from the C. reinhardtii iron hydrogenase gene are used to amplify iron hydrogenase genes from other microbial genomes. In this method the PCR template is genomic DNA, a cDNA library, or RNA for use in RT-PCR. The sequences isolated from each microbe are mixed and put through a shuffling procedure.

In one embodiment, a plurality of genes is identified from the differential expression profile as upregulated when C. reinhardtii cells are exposed to conditions more conducive to the generation of hydrogen. Sequence information from these genes is used to generate probes and PCR primers corresponding to the sequences. A plurality of green algae species, originally isolated from disparate geographic locations, are cultured under conditions more conducive to the generation of hydrogen. A cDNA library from each green algae species is generated and utilized for the isolation of sequences corresponding to each of the sequences identified from C. reinhardtii as differentially regulated using the probes corresponding to the upregulated C. reinhardtii sequences. The isolated gene sequences are used for shuffling.

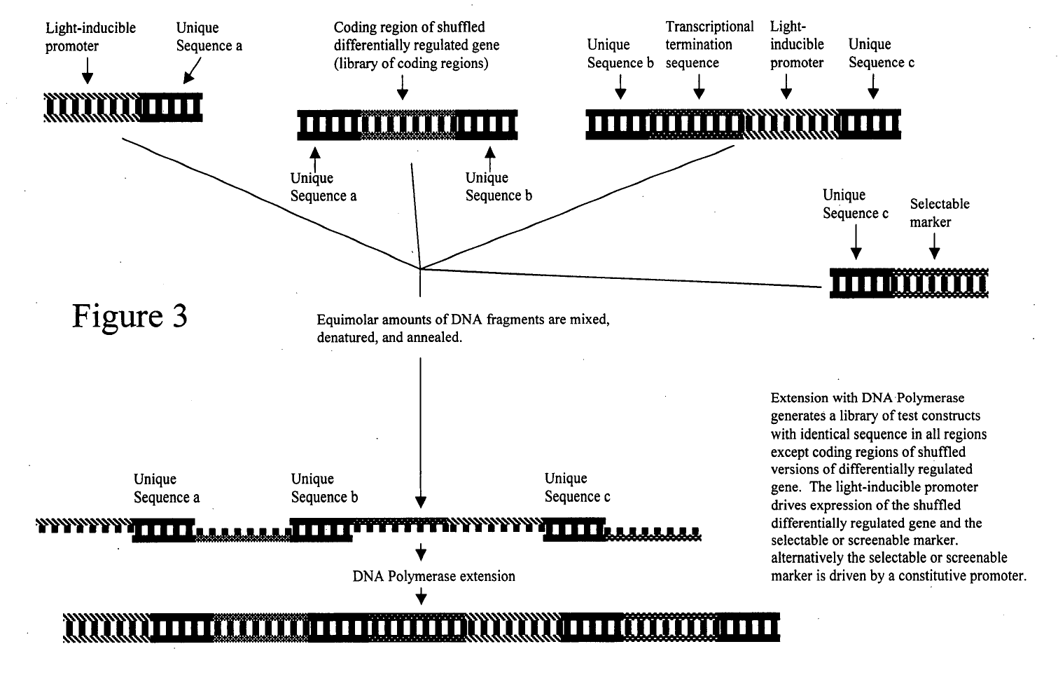

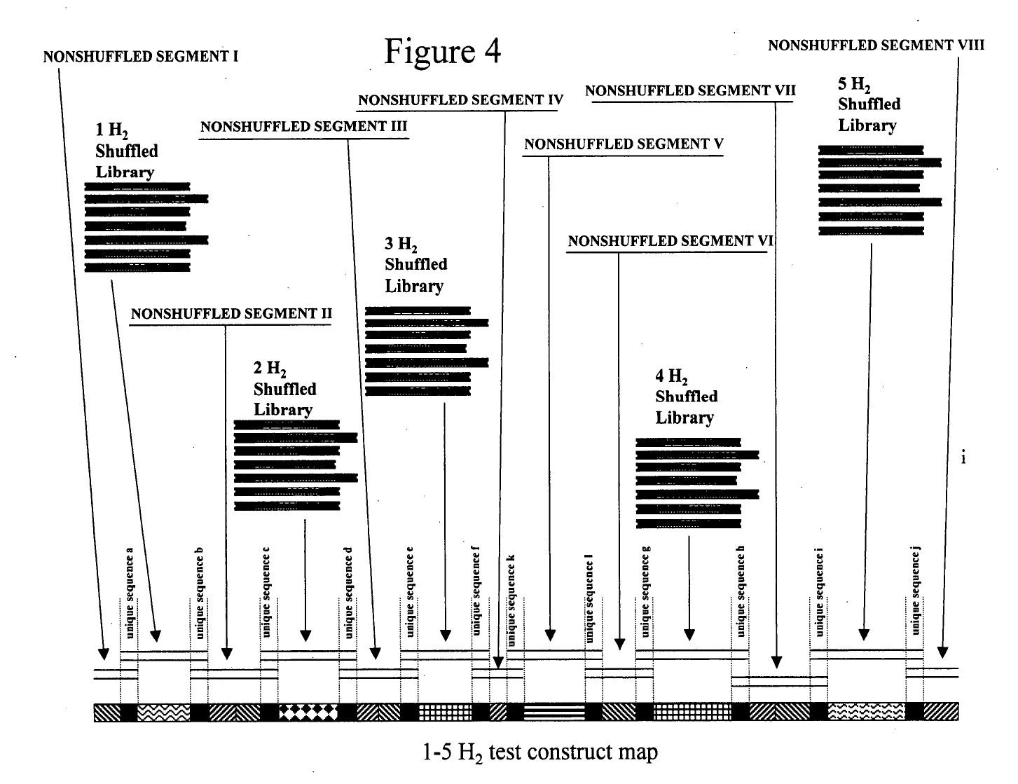

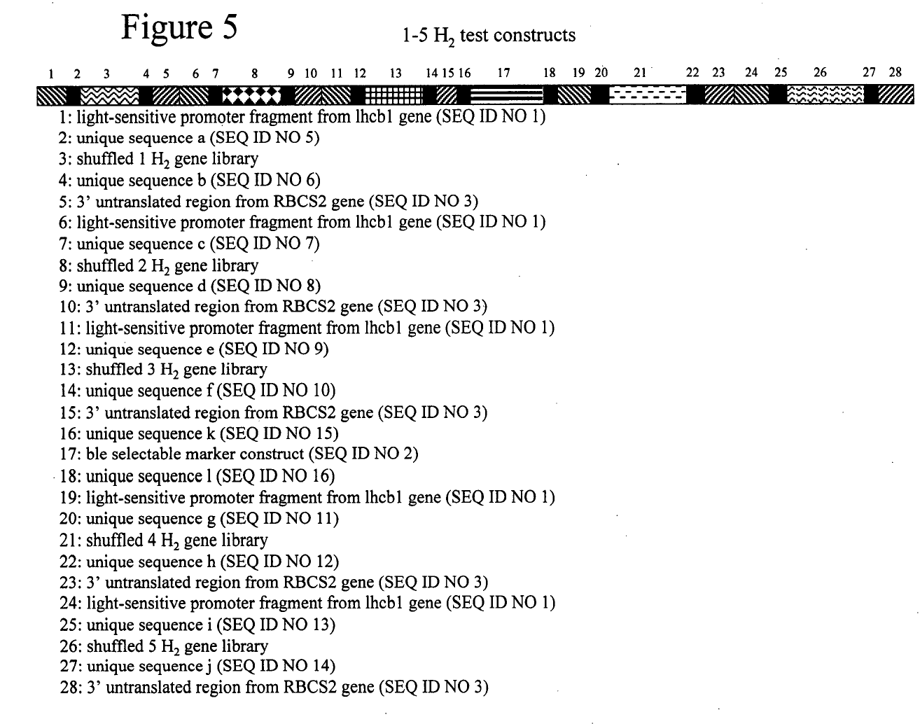

In one embodiment, the plurality of genes is shuffled in reactions containing synthetic chimeric oligonucleotides. The chimeric oligonucleotides possess on one end sequence corresponding to either the 5′ or 3′ end of the coding region of genes included in the shuffling reaction. On the other end these chimeric oligonucleotides contain heterologous sequence, such as unique sequences not found in the genes that are shuffled or in the genome of the hydrogen producing microbe. The unique sequences are used to connect different components of DNA constructs containing mutagenized nucleic acid sequences (FIG. 3). Other chimeric oligonucleotides contain sequences corresponding to (a) a promoter sequence and (b) a unique sequence. The sense and antisense strands of unique sequences are used to join mutagenized nucleic acid sequences with promoter sequences and other types of sequence heterologous to the mutagenized nucleic acid sequences. For example, a promoter sequence imparts transcriptional activation to a downstream mutagenized nucleic acid sequence when placed in a Chlamydomonas cell that is exposed to light (Hahn, Curr Genet (1999) January; 34(6):459-66; Loppes, Plant Mol Biol 2001 January; 45(2):215-27; Villand, Biochem J 1997 Oct. 1;327 (Pt 1):51-7). Other light-inducible promoter systems may also be used, such as the phytochrome/PIF3 system (Shimizu-Sato, Nat Biotechnol 2002 October; 20(10):1041-4). Alternatively or in addition, the promoter sequence imparts transcriptional activation to a downstream gene when placed in a Chlamydomonas cell that is exposed to light and heat (Muller, Gene (1992) February 15; 111(2): 165-73; von Gromoff, Mol Cell Biol (1989) September; 9(9):3911-8). Alternatively the promoter sequence imparts transcriptional activation to a downstream gene when an exogenous molecule is added to the culture media using receptors not present in the wild-type cell such as receptors for estrogen, ecdysone, or others (Metzger, Nature 1988 Jul. 7; 334(6177):31-6; No, Proc Natl Acad Sci USA 1996 Apr. 16; 93(8):3346-51). Alternatively the promoter sequence imparts transcriptional activation in a constitutive fashion, such as the promoter of the psaD gene (Fischer, WO 01/48185). When the shuffled gene fragments are annealed and subjected to primerless PCR, the 5′ and 3′ ends of the shuffled coding regions anneal to chimeric oligonucleotides that in turn anneal to other heterologous sequences such as promoters and 3′ untranslated regions that enhance expression levels (Lumbreras, Plant J (1998) 14(4): 441-447). The 5′ end of every coding sequence created through the shuffling procedure is annealed to a chimeric oligonucleotide corresponding to a unique sequence. The unique sequence in turn anneals to a nonshuffled segment of DNA containing a promoter sequence (FIGS. 3, 4). Unique sequences are thus used to attach components of DNA constructs to each other that do not possess sequence homology. In addition, chimeric oligonucleotides are included that possess homology to internal parts of the coding region of shuffled genes as well as intron sequences to direct the insertion of intron sequences into coding regions to aid in effective expression levels (Lumbreras, Plant J (1998) 14(4): 441-447).

Chimeric oligonucleotides may be used to connect any part of a nucleic acid construct to another in shuffling protocols. Intron, transcriptional terminator, splice sequences, centromeres, selectable and screenable markers are all introduced into nucleic acid constructs through annealing these elements to chimeric oligonucleotides that contain heterologous sequence, followed by promoterless PCR protocols.

In one embodiment, libraries of individually shuffled homologous genes with unique sequences at each end are mixed with other distinct libraries of individually shuffled homologous genes that also contain unique sequences at both 5′ and 3′ ends. Also mixed with the shuffled libraries of coding sequences are nonshuffled segments containing structural and functional DNA elements such as promoters, 3′ untranslated regions, and screenable or selectable markers. The nonshuffled segments of DNA are also flanked with unique sequences, all of which are identical to unique sequences flanking certain shuffled sequences. All of the molecules are denatured, annealed, and subjected to a primerless PCR reaction in which “sense” and “antisense” unique sequences anneal to each other and prime extension by a polymerase, thus placing each shuffled and nonshuffled sequence into its desired place on the resulting DNA construct. The resulting library of DNA constructs contains shuffled genes operatively linked to promoter sequences. (FIGS. 3, 4)

In one embodiment chimeric oligonucleotides contain sequence corresponding to genes being shuffled and heterologous sequence corresponding to introns, splice sequences, centromeres, selectable markers, unique sequences or other linker sequences designed to serve as structural parts of the construct. The design of the DNA construct using these chimeric oligonucleotides creates a functional DNA construct directly from the shuffling procedure. Any desired component of a DNA construct is included through the use of chimeric oligonucleotides that connect heterologous sequences of the construct during the annealing step. For instance, the inclusion of a light-inducible promoter allows the shuffled versions of differentially regulated genes to be activated by light rather than the conditions more conducive to the generation of hydrogen

In one embodiment each DNA construct in the library of DNA constructs contains a plurality of shuffled genes that possess sequence homology to a set of upregulated differentially regulated genes. Each coding region has an upstream light-inducible promoter and a downstream untranslated transcriptional terminator sequence. Each coding region contains an intron and functional splice sequences. Each construct contains at least one selectable marker. Constructs optionally also contain other functional or structural sequences. For example, centromeres or other sequences employed for the purpose of allowing the construct to be retained in dividing cells and/or sequences that aid in integration of the construct into random or specific regions of the host genome are included in the construct. In other embodiments the promoter is constitutive or is inducible by a stimulus other than light, such as the addition of a small molecule to the culture media.

In one embodiment, DNA constructs are used to turn off or downregulate the expression of differentially regulated genes that are downregulated when cells are exposed to conditions more conducive to the generation of hydrogen. These constructs work through the use of antisense and/or RNA interference methods. In this embodiment, a DNA construct containing at least one antisense sequence operatively linked to a promoter is used to transform cells for the purpose of downregulating the expression of a gene or genes that are naturally downregulated when cells are exposed to conditions more conducive to the generation of hydrogen. For example, in Chlamydomonas, antisense inhibition is utilized to effect a drop in expression of the targeted gene (Schroda, Plant Cell (1999) June; 11(6):1165-78). Alternatively, an RNA interference (RNAi) construct is used (Fire, Nature (1998) February 19; 391 (6669):806-11; Fuhrmarn, J Cell Sci (2001) November; 114(Pt 21):3857-63). In one embodiment, DNA constructs are synthesized that contain shuffled sequences corresponding to genes upregulated when cells are exposed to conditions more conducive to the generation of hydrogen and RNAi sequences corresponding to genes downregulated when cells are exposed to conditions conducive to the generation of hydrogen. Both the shuffled sequences and the RNAi sequences are functionally coupled to promoters that are activated by the same stimuli, different stimuli, or are constitutively active.

In one embodiment genes downregulated when cells are exposed to conditions less conducive to the generation of hydrogen are removed from the genome through gene targeting methods that utilize homologous recombination (Naver, Plant Cell 2001 December; 13(12):2731-45).

In one embodiment molecules that interfere with the function of proteins that are encoded by genes downregulated when cells are exposed to conditions more conducive to the generation of hydrogen are either placed in the culture media or synthesized by proteins encoded by transgenes inserted into cells.

In one embodiment the DNA constructs containing shuffled upregulated differentially regulated genes contain genes encoding screenable or selectable markers at each end of a linear DNA construct. For example, at one end of the construct is a gene encoding a fluorescent protein optimized for use in Chlamydomonas (Fuhrmann, Plant J (1999) August; 19(3):353-61). At the other end is a gene encoding a selectable marker gene that imparts resistance to an antibiotic (Stevens, Mol Gen Genet (1996) April 24; 251(1):23-30). Between the fluorescent protein and the antibiotic resistance gene are shuffled versions of genes upregulated when cells are exposed to conditions more conducive to the generation of hydrogen or are involved in the hydrogen production pathway, such as ferredoxin, catalase, isoamylase, malate dehydrogenase, 14-3-3 protein, enolase, aldolase, ribosomal protein S8, ribosomal protein L17, ribosomal protein S18, ribosomal protein L37, ribosomal protein L12, ribosomal protein S15, iron-hydrogenase, and components of the photosystem I, photosystem II and cytochrome b₆-f complexes. Components of the photosystem I and II complexes are disclosed, for example, in Elrad, Curr Genet. 2003 December 2. Hydrogen can be produced in C. reinhardtii for example, by pathways that opetare in light and dark. Mutagenized genes from either pathway can be assayed using the methods disclosed herein. Cells are transformed with the library of constructs and are cultured in media containing the antibiotic. Cells that survive under these culture conditions are run through a fluorescence activated cell sorter that plates each cell expressing the green fluorescent protein onto a grid pattern on solid media or into multiwell plates containing liquid growth media containing the antibiotic. Colonies are screened or selected for the ability to generate an increased amount of hydrogen. Cells that retain both markers have also retained all the sequence in the DNA construct between the two markers. Large numbers of genes may be placed between the two markers. Preferably only cells that retain both markers are put through screening or selection procedures.

In one embodiment the mutagenized nucleic acid sequence encodes an iron hydrogenase protein and the cell is a green algae species such as C. reinhardtii. Further, the mutagenized nucleic acid sequence is generated by mutagenizing a C. reinhardtii iron hydrogenase gene at at least one amino acid position. The mutagenized nucleic acid sequence is used in a construct to transform the cell. Preferably, the iron hydrogenase protein retains the capacity to functionally interact with a ferredoxin or other electron donor in the cell. “Functionally interact” means that a ferredoxin or other electron donor transfers electrons to the hydrogenase protein. Preferably the sequence change(s) caused by the mutagenesis of the C. reinhardtii iron hydrogenase gene does not disrupt the functional interaction between the protein encoded by the mutagenized C. reinhardtii iron hydrogenase gene and ferredoxin or another electron donor. Preferably the mutagenesis creates an oxygen tolerance phenotype without disrupting the functional interaction with a ferredoxin. More preferably, the mutagenesis creates an oxygen tolerance phenotype while enhancing the functional interaction with a ferredoxin. An example of an enhanced functional interaction with ferredoxin is a functional interaction that allows more electrons to be shuttled from the endogenous ferredoxin to the mutagenized iron hydrogenase per unit time under than with the non-mutagenized C. reinhardtii iron hydrogenase. An enhanced functional interaction can also be screened or selected for by mutagenizing the ferredoxin, as described in Example 2.

Providing Mutagenized Nucleic Acid Sequences Corresponding to Genes Known to be Involved in a Hydrogen Production Pathway

Wild type iron hydrogenase genes are preferred mutagenesis targets with which to generate mutagenized nucleic acid sequences. Mutagenesis preferably alters characteristics such as oxygen tolerance while not altering characteristics such as the ability to functionally interact with ferredoxin.

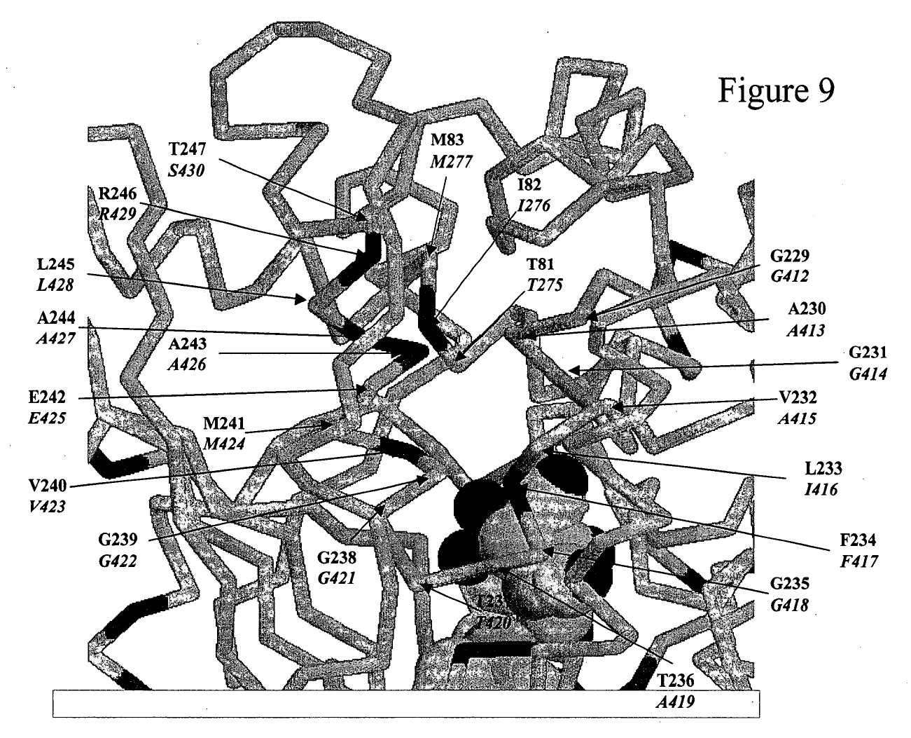

In one embodiment, the C. reinhardtii iron hydrogenase gene is mutated to alter amino acid residues in and near the gas channel. The gas channel is a section of iron hydrogenases, depicted in FIG. 9, that allows newly formed hydrogen molecules to leave the protein. Oxygen irreversibly inactivates the active site of iron hydrogenases by entering the active site through the gas channel (for background see Ghirardi, Appl Biochem Biotechnol (1997) 63-65: 141-151). Because hydrogen molecules are smaller than oxygen molecules, narrowing the gas channel using methods deiclosed herein provides iron hydrogenases that are not inactivated by oxygen. Preferably, substitutions of residues that are in and near the gas channel generate side chains that are of higher molecular weight or are longer than the side chain at that position in the wild type protein. Such substitutions are preferable because they narrow the gas charnel and block the entry of oxygen into the active site. As one nonlimiting example, residues in the highly conserved X¹X²X³X⁴X⁵X⁶GGVMEAAX⁷R segment can be mutated. This segment forms a turn followed by an alpha helix. The F corresponds to Phe234 in the wild type C. reinhardtii iron hydrogenase. The X residues are highly variable between iron hydrogenase from different species. For example, the X⁴X⁵X⁶ residues are GVT, GAT, GVS, GNS, CAS, and numerous other sequences in different iron hydrogenases. Nonetheless, members of the iron hydrogenase family usually have a G as the first residue of this triplet. Although the GGVMEAA amino acid motif is highly conserved among members of the iron hydrogenase family, there are some iron hydrogenases that have variant sequences corresponding to this motif For example, the D. fructosovorans iron hydrogenase (GenBank Accession number D57150) has the sequence GGVIEAA. Thus, even highly conserved motifs that surround the gas channel are tolerant of change.

Other amino acid motifs also form secondary structures near the gas channel. For example, the ADX⁸TIX⁹EE motif is in close contact with the channel. In particular, the T, I and X⁹ residues are near the channel.

In one embodiment, highly variable amino acids are subjected to saturation mutagenesis. In another embodiment, highly variable amino acids are substituted with any amino acid that is of a higher molecular weight hat the wild type amino acid at that position in either of the C. reinhardtii iron hydrogenases. In another embodiment, variable amino acids in either of the C. reinhardtii iron hydrogenases are substituted with amino acids that are found in the corresponding position in iron hydrogenases from different species. In yet another embodiment, the X¹X²X³X⁴X⁵X⁶GGVMEAAX⁷R motif is mutated in either of the C. reinhardtii iron hydrogenases referred to as hydA and hydB (Forestier, Eur J. Biochem. 2003 July; 270(13):2750-8), wherein some of the X residues are substituted with amino acids that are found in the corresponding position in iron hydrogenases from different species while other X residues are substituted with residues that are no found in any known species. In one embodiment residues X¹X²X³ are from species 1, residues X⁴X⁵X⁶ are from species 2, and residue X⁷ is from species 3, where these X residues are placed in the context of a C. reinhardtii iron hydrogenase protein, and where none of species 1, 2, or 3 is C. reinhardtii. The methods provided herein include mutagenizing genes by substituting any segment of a protein sequence into another protein sequence, including genes encoding iron and nickel-iron hydrogenase proteins. Preferable lengths for segments include 2, 3, 4, 5, 6, 7, 8, 9, 10, 11, 12, 13, 14, 15 or more amino acids. Of course, the methods provided also included substituting single amino acids from one species into the proteins of another species at a particular position as well as substituting amino acids that do not correspond to amino acids of another species at a particular position.

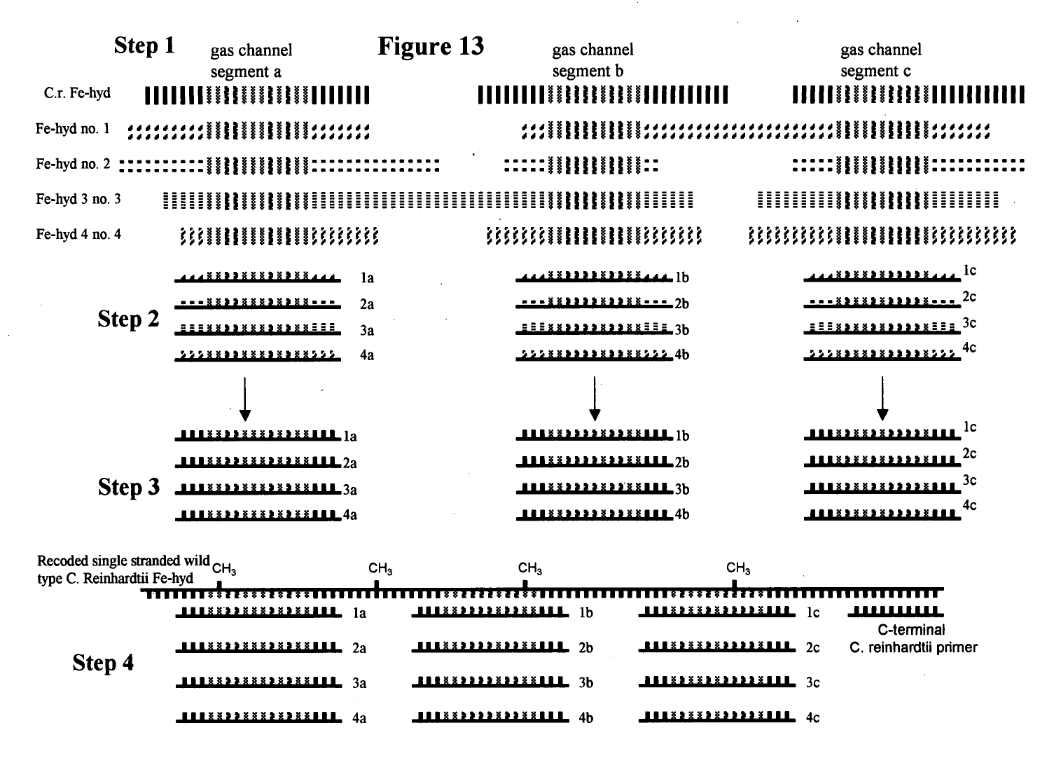

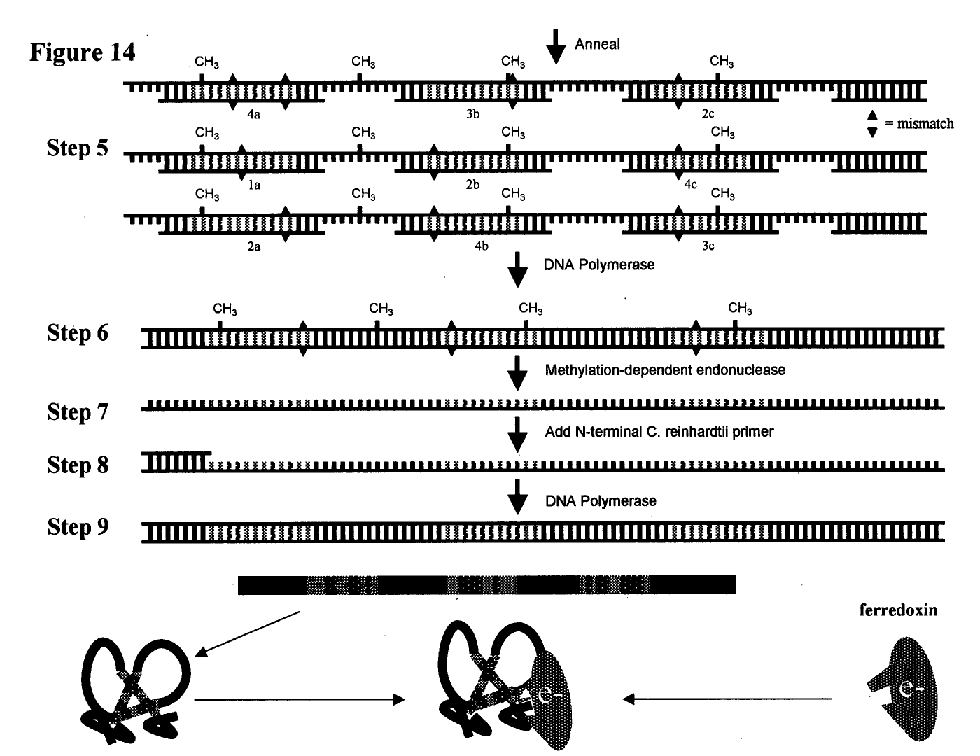

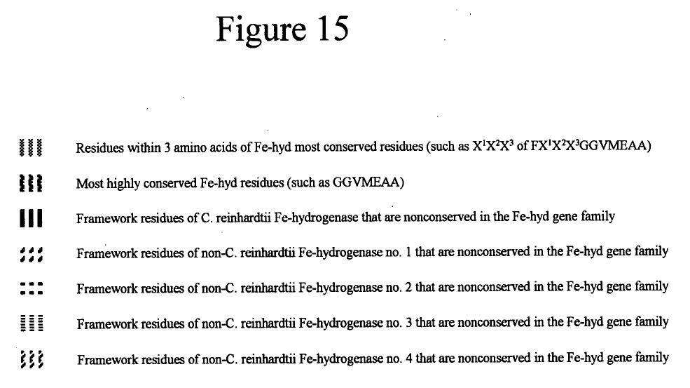

In another embodiment, gene reassembly of the iron hydrogenase is performed. Sections of the C. reinhardtii iron hydrogenase active site region that are both highly conserved and correspond to the gas channel are used to construct a library of iron hydrogenase genes, depicted schematically in FIG. 13. In step 1, the library of iron hydrogenase amino acid sequences from SEQ ID NOs: 1-112 was aligned using sequence manipulation software (DS Gene, Accelyrys Inc., San Diego, Calif.). The key in FIG. 15 shows the identity of amino acids from step 1 and codons from steps 2-9. All bars in steps 2-9 correspond to codons that encode the amino acids from the bars of step 1. Each bar in steps 2-9 therefore depicts a codon triplet of oligonucleotide sequence. In step 2, conserved amino acid segments were identified in the alignment and reverse-translated into single stranded oligonucleotide sequences utilizing C. reinhardtii most preferred codons. In step 3, 3 codons encoding amino acids flanking these highly conserved gas channel sequences were re-written as the C. reinhardtii flanking sequence of the oligonucleotides. Even though these oligonucleotides encode different gas channel segments from the C. reinhardtii iron hydrogenase, the combination of the recoding process and the substitution of 3 flanking C. reinhardtii codons generates enough nucleotide similarity that these oligonucleotides anneal to a complementary strand encoding the recoded, wild-type C. reinhardtii iron hydrogenase. In step 4, the set of recoded oligonucleotides corresponding to diverse gas channel segments are annealed to a single stranded DNA molecule that encode C. reinhardtii Iron hydrogenase protein using the same C. reinhardtii most preferred codons. In addition, oligonucleotides corresponding to wild type C. reinhardtii amino acid sequences with single residue substitutions designed to narrow the gas channel can also be included in the annealing reaction. A C. reinhardtii C-terminal primer is also added to the annealing reaction. The single stranded molecule is generated by isolating the gene from a plasmid grown in a methylating host cell, followed by denaturation and separation of the strands by HPLC or other standard procedures, as described for example in U.S. Pat. No. 6,361,974. As shown in step 5 of FIG. 14, different combinations of segments anneal to each full length complementary strand. Addition of DNA Polymerase in step 6 extends the annealed oligonucleotides, creating a library of double stranded hybrid molecules with mismatches at “context” residue positions. Preferably the DNA Polymerase is exonuclease-deficient to prevent it from degrading parts of annealed primers in its path as it extends between annealed primers. In step 7, the methylated strands are digested using a methylation-sensitive endonuclease, as described for example in U.S. Pat. No. 6,361,974. In steps 8-9, N-terminal C. reinhardtii primer and DNA Polymerase are added to the library of novel iron hydrogenase molecules. As an alternative to methylation, the C-terminal primer shown first in step 4 can be biotinylated, and the mismatched wild type and library strands can be separated in step 7 by denaturation and separation using immobilized streptavidin.

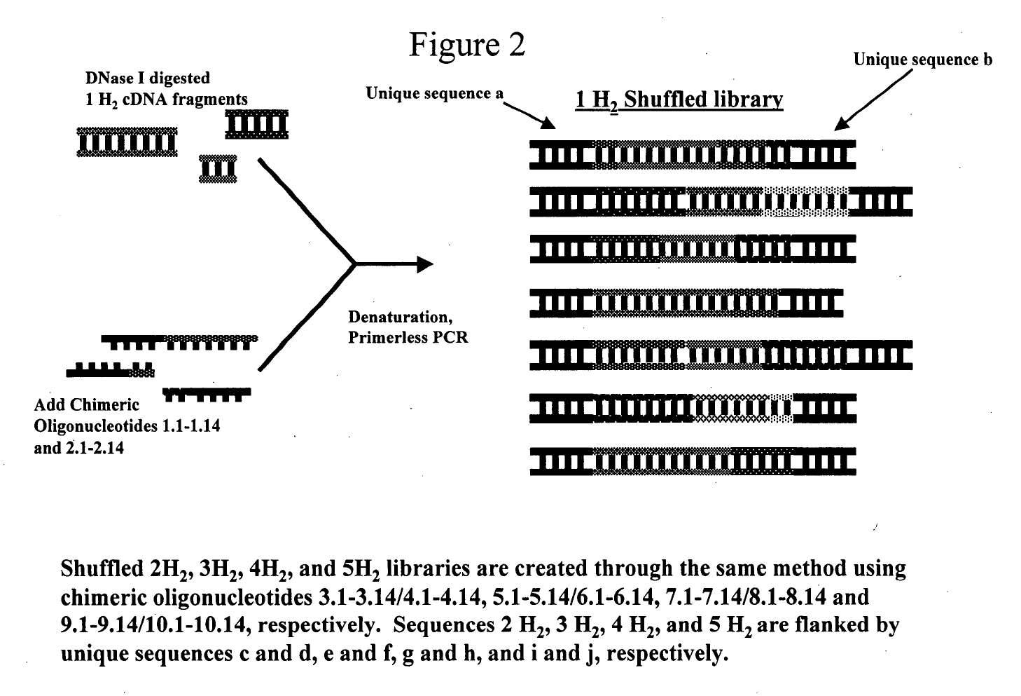

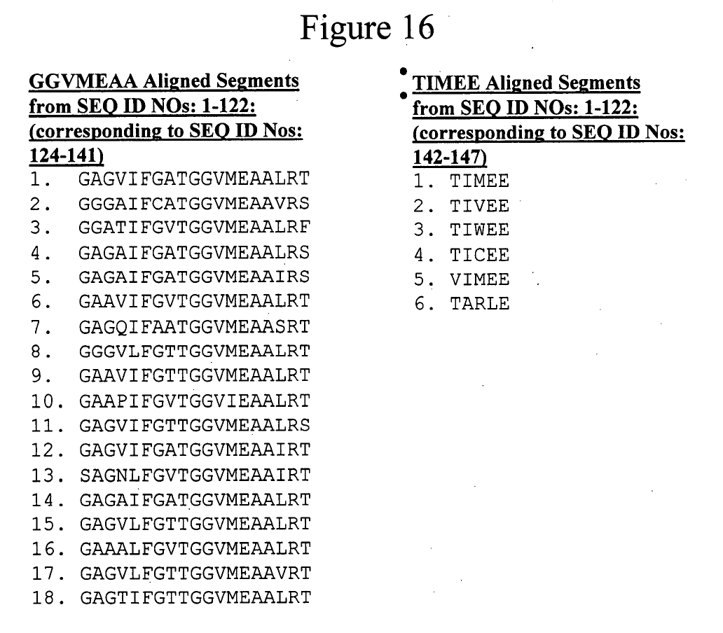

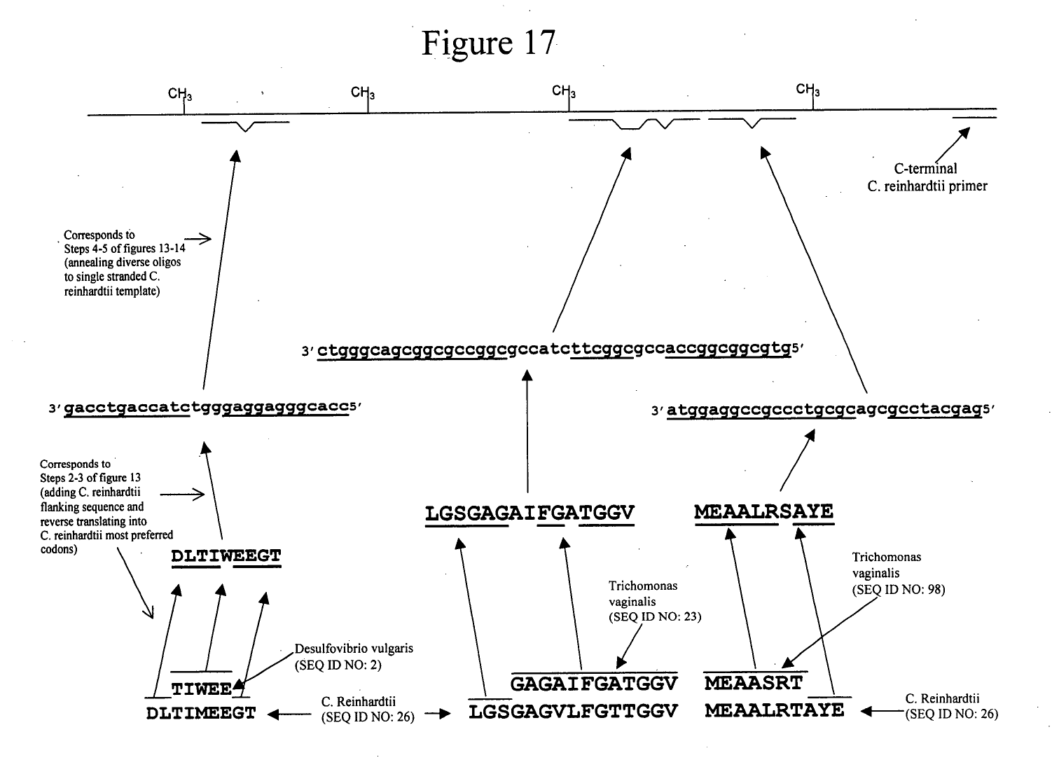

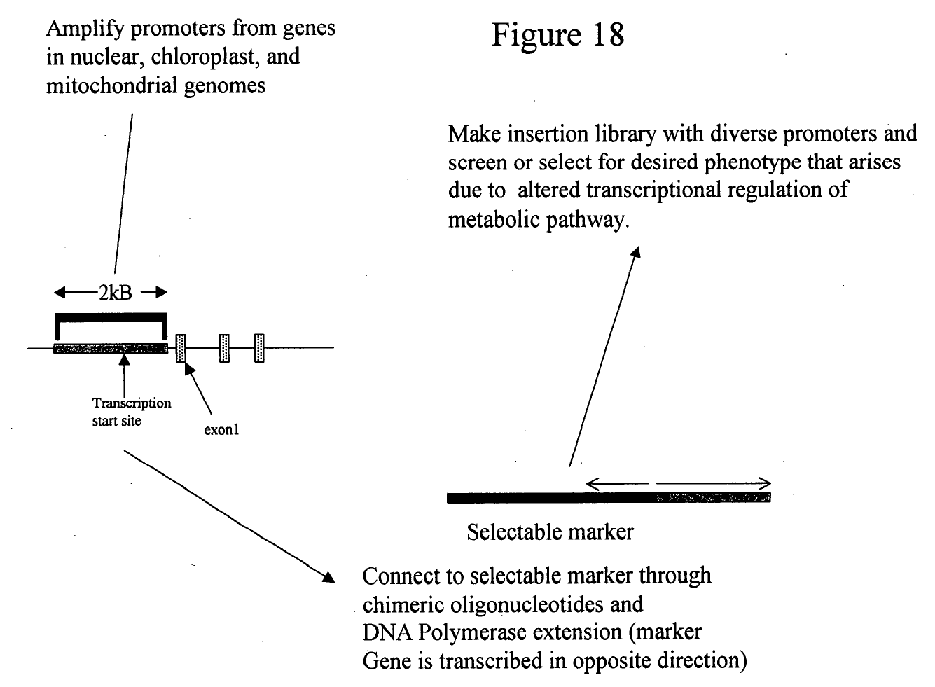

The result of the above process is a library of double stranded iron hydrogenase sequences that have random combinations of functional gas channel segments and C. reinhardtii framework/hinge regions. The population is cloned into C. reinhardtii cells and assayed as described in previous sections. This method does not use an exonuclease such as mung bean nuclease. No single stranded fragments that anneal to the methylated strand have partially overlapping binding sites. The advantage of this method of creating mutagenized nucleic acid sequences is that the library can be tested for oxygen tolerance but preserves C. reinhardtii framework/hinge domains that functionally interact with ferredoxin than a library made using other gene reassembly procedures such as the procedure shown in FIGS. 2-3 that involves reassembly of the entire gene sequence. In a preferred embodiment, single stranded nucleotide molecules, using C. reinhardtii most preferred codons, encoding segments or fragments of segments depicted in FIG. 16 are used in the procedure. Although FIG. 17 depicts one possible arrangement of three diverse oligonucleotides that can be annealed to a single stranded wild type sequence, mixing oligonucleotides corresponding to each of the identified gas channel segments from SEQ ID Nos: 124-147 that have C. reinhardtii flanking codons produces a large number of possible combinations of library sequences. Each possible combination corresponds to a different gas channel architecture that can be tested for the ability to allow flow of hydrogen but not oxygen.

Alternatively, other genes involved in a hydrogen production pathway are mutagenized. Examples of these genes are recited elsewhere in this application. As one example, genes encoding light antenna complexes are mutagenized and inserted into cells. For example, one or more genes from a light harvesting complex of C. reinhardtii, such as those disclosed in Teramoto, Plant Cell Physiol. 2001 August; 42(8):849-56. (corresponding to GenBank accession numbers M24072, AF104630, AF104631, AB050007, X65119), and Elrad, Curr Genet. 2003 December 2 (lhcbm1, lhcbm2, lhcbm3, lhcbm4, lhcbm5, lhcbm6, lhcbm8, lhcbm9, lhcbm11, lhca1, lhca2, lhca3, lhca4, lhca5, lhca6, lhca7, lhca8, lhca9, lhcb4, lhcb5, lhcq, 11818-111818-2, elip1, elip2, elip3, elip4, and elip5) are mutagenized and used to transform C. reinhardtii. Transformants are screened or selected for the ability to produce an increased amount of hydrogen under conditions such as high light, low light, sunlight, or light of a certain wavelength range. For example, segments of amino acids from antenna proteins of one species are inserted into antenna proteins from C. reinhardtii. The mutagenized nucleic acid sequence is then inserted into C. reinhardtii cels and the transformed cells are screened or selected for the ability to live and/or produce hydrogen in the presence of photoautotrophic media and light. In one embodiment the light is of a wavelength that wild type C. reinhardtii antenna proteins are not capable of harvesting.

In another embodiment, an siRNA construct is used to transform a cell, where the siRNA construct is designed to reduce or eliminate the expression of a gene that reduces the photosynthetic efficiency or rate. For example, the C. reinhardtii lhcbm1 gene is reduced or eliminated in expression using siRNA (sequence of lhcbm1 in Elrad, Plant Cell. 2002 August; 14(8): 1801-16).

In one embodiment, cell transformed with mutagenized antenna genes are cultured in the presence of light outside the normal wavelength range of the starting strain. For example, genes encoding purple bacteria antenna complexes are transformed into green algae such as C. reinhardtii. The genes include preferably only the most preferred codon of C. reinhardtii for each amino acid. Preferably, bacteriochlorophyll molecules are present in the cells, either synthesized by enzymes also present in the C. reinhardtii cell or added exogenously to the culture media. The cells are cultured in photoautotrophic media under light of wavelengths that wild type green algae are not capable of capturing, such as 770-920 nm. Narrow ranges can be used as well, such as 800-900 nm. In one embodiment, the a peptides of Rs. rubrum, Rb sphaeroides, and Rb. capsulatus are reverse translated into C. reinhardtii most preferred codons (see sequences from Davis, Biochemistry. 1997 March 25; 36(12):3671-9.). These α peptide genes, encoding amino acids only in C. reinhardtii most preferred codons, are shuffled. The β peptides from the above three organisms, also as shown in Davis, are also reverse translated into C. reinhardtii most preferred codons and shuffled. The shuffled α and β peptides are cloned into expression vectors and used to transform C. reinhardtii. Preferably the α and β peptide sequences also include targeting domains that cause the expressed proteins to be embedded in light harvesting complexes of the C. reinhardtii thylakoid membrane. The transformed population is cultured under light of a wavelength above 700 nm, preferably above 750 nm, more preferably above 800 nm. Surviving strains are then assayed for hydrogen production in light of a wavelength above 700 nm, preferably above 750 nm, more preferably above 800 nm.

In another embodiment, shuffling is performed using nucleic acid molecules encoding nickel-iron hydrogenase proteins, such as those in SEQ ID NOs: 113-122. Because these Ni—Fe hydrogenases are made of alpha and beta subunits, preferably the nucleic acid molecules encoding segments of each protein are shuffled in separate reactions. The shuffled libraries are expressed in cells that possess Ni-Iron hydrogenaserogenase maturation enzymes, such as E. coli.

Transforming Cells With Mutagenized Nucleic Acid Sequences

Cell transformation methods and selectable markers for photosynthetic bacteria and cyanobacteria are well known in the art (Wirth, Mol Gen Genet 1989 March; 216(1):175-7; Koksharova, Appl Microbiol Biotechnol 2002 February; 58(2): 123-37; Thelwell). Transformation methods and selectable markers for use in bacteria are well known (Maniatis et al. (1989) Molecular Cloning: A Laboratory Manual Cold Spring Harbor Laboratory).

In green algae, the nuclear, mitochondrial, and chloroplast genomes are transformed through a variety of known methods. (Kindle, J Cell Biol (1989) December; 109(6Pt 1):2589-601; Kindle, Proc Natl Acad Sci USA (1990) February; 87(3): 1228-32; Kindle, Proc Natl Acad Sci U S A (1991) March 1; 88(5):1721-5; Shimogawara, Genetics (1998) April; 148(4):1821-8; Boynton, Science (1988) June 10;240(4858):1534-8; Boynton, Methods Enzymol (1996) 264:279-96; Randolph-Anderson, Mol Gen Genet (1993) January; 236(2-3):235-44).

Selectable markers for use in Chlamydomonas are known, including but not limited to markers imparting spectinomycin resistance (Fargo, Mol Cell Biol (1999) October; 19(10):6980-90), kanamycin and amikacin resistance (Bateman, Mol-Gen Genet (2000) April; 263(3):404-10), zeomycin and phleomycin resistance (Stevens, Mol Gen Genet (1996) April 24; 251(1):23-30), and paromycin and neomycin resistance (Sizova, Gene (2001) October 17; 277(1-2):221-9).

Screenable markers are available in Chlamydomonas, such as the green fluorescent protein (Fuhrmann, Plant J (1999) August; 19(3):353-61) and the Renilla luciferase gene (Minko, Mol Gen Genet (1999) October; 262(3):421-5). Fluorescent proteins are also available for prokaryotic organisms.

In one embodiment, libraries of gene sequences that encode proteins that physically interact are shuffled. Nucleic acid constructs are used for transformation procedures that contain a shuffled version of each gene. Sequences that encode proteins that interact in ways more conducive to the generation of hydrogen are screened or selected for. By mutagenizing sequences encoding proteins that physically interact, more favorable interactions are generated that lead to the production of increased levels of hydrogen. Examples of such proteins in the hydrogen production pathway that physically interact are iron-hydrogenase/ferredoxin and proteins in the photosystem I, photosystem II, and cytochrome b₆-f complexes. It is advantageous but not necessary to use pairs or sets of genes that encode proteins that physically interact from the same organisms. Providing interacting pairs or sets in the shuffling procedure increases the odds of obtaining favorable functional interactions due to the possibility of obtaining shuffled sequences on the same test construct that contain complementary interaction domains from the same organism, regardless of the sequence flanking either side of the interaction domain in any of the sequences.

In one embodiment, a library of sequences corresponding to at least one mutagenized nucleic acid sequence derived from a differentially regulated gene is inserted into cells through a transformation procedure. Cells that have been transformed with the library are then put through a screening or selection process in which the cells are assayed for the ability to generate an increased amount of hydrogen when compared to the non-transformed strain or the strain transformed with only vector and/or screenable/selectable marker sequences.

Screening or Selecting for a Cell that Generates an Increased Amount of Hydrogen

Cells are screened for the ability to produce hydrogen by a variety of methods. One method involves the use of gas chromatography, which is a well known method of detecting gases such as hydrogen. An intake device attached to the gas chromatography machine is placed in close enough proximity to the cell culture container or plate that it can detect, and preferably quantify, the hydrogen produced by the cells (U.S. Pat. No. 5,100,781).

Oxygen content may be manipulated in the culture container. The amount of oxygen in the culture container may be directly adjusted through gas exchange or indirectly by allowing or inducing the water-splitting mechanism of photosynthesis. The oxygen content, like all other culture parameters, may be manipulated throughout the culture period or held constant. The presence of some amount of oxygen is preferred if MNZ is added to the culture media Preferred hydrogenase genes are capable of catalyzing the production of hydrogen in the presence of oxygen. A preferable amount of oxygen in a culture of commercially deployed cells for hydrogen production is an atmospheric level such as approximately 21%. Several rounds of screening or selection may be performed in which the oxygen content of the culture container may be increased between each successive round while hydrogen production is assayed. For example, a culture is exposed to 5% oxygen in the first screening or selection round, 10% oxygen in the second screening or selection round, 15% oxygen in the third screening or selection round, and 20% oxygen in the fourth screening or selection round. Other levels of oxygen that can be tested include more than 0.5%, more than 5.0%, more than 10%, more than 15%, approximately 21%, more than 21%, more than 25%, more than 30% or more than 35%.

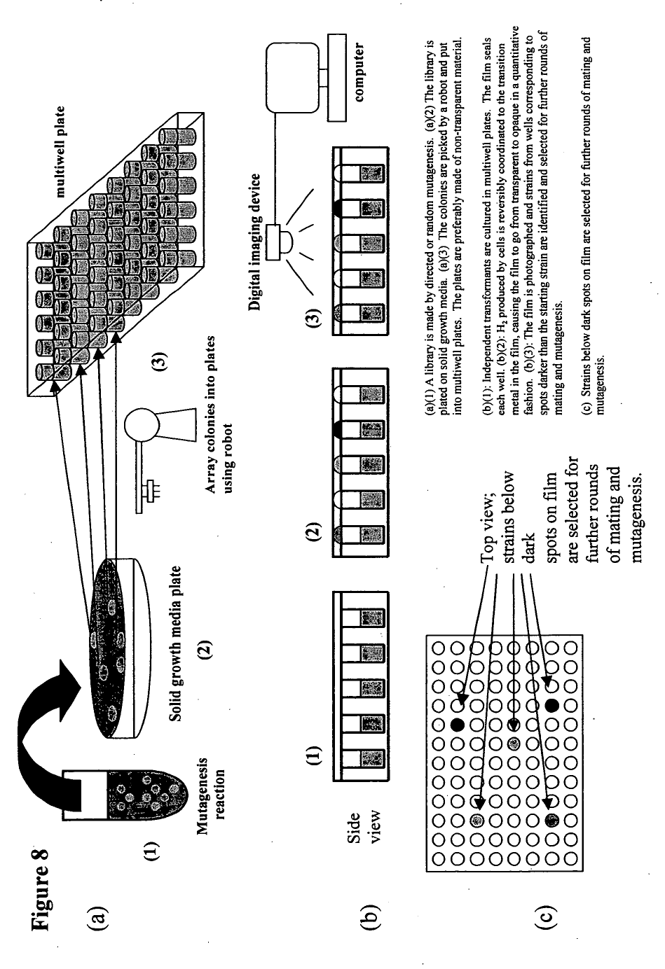

In one embodiment, the screening assay is a chemochromic film that turns from transparent to opaque in the presence of hydrogen. The assay is performed by placing films over arrays of multiwell plates containing libraries of C. reinhardtii transformants. As shown in FIG. 8, independent transformants are cultured in multiwell plates. The film seals each well. Hydrogen produced by cells is reversibly coordinated to the transition metal in the film, causing the film to go from transparent to opaque in a quantitative fashion. The film is photographed with digital imaging equipment and cells from wells corresponding to spots darker than the starting strain are selected for further rounds of mutagenesis.

The assay is performed using a platform in which a variety of parameters are manipulated. The platform contains an enclosed chamber in which multiwell plates are exposed to a controlled gas environment. Lights are positioned over the chamber such that daylight/nighttime conditions may be mimicked. The temperature of the chamber may be manipulated corresponding to colder nighttime temperatures followed by warmer daytime temperatures. The platform allows the directed evolution procedure to create novel microbe strains that are best suited for commercial deployment. For example, in one embodiment strains that can produce hydrogen for hundreds of hours using constant light at a constant temperature are assayed for; in a second embodiment strains capable of producing large amounts of hydrogen during a warmer 12 hour light period after being exposed to a colder 12 hour dark period are assayed for. Strains produced by the second embodiment are best suited for commercial deployment because they are best able to conserve energy at night when the photosynthetic electron transport chain is not functional.

In one embodiment, the hydrogen production assay mimics commercial deployment conditions through the use of deep-well plates made from non-transparent plastic material. When mutants are assayed for hydrogen production, the light available to the cells comes only from directly above the plates, mimicking conditions under which cells in a large bioreactor are exposed to light. Mutations that attenuate phototaxis (swimming towards light) under bright light conditions (but not dim conditions) prevent cells from accumulating at the surface of the media and blocking photons from penetrating deeper into the media Mutations in the antenna complexes also enhance photon utilization efficiency.

In one embodiment, cells transformed with mutagenized nucleic acid sequences are cultured under conditions in which gas in the culture container comprises 5% oxygen. Cells that generate an increased amount of hydrogen are recovered and mutagenized nucleic acid sequences are recovered from the cells. The mutagenized nucleic acid sequences are put through a further mutagenesis round and are used to transform cells. The transformed cells are cultured under 21% oxygen. Mutagenized nucleic acid sequences corresponding to differentially regulated genes whose wild type sequence encodes proteins that do not function or minimally function in atmospheric oxygen levels, such as the C. reinhardtii iron hydrogenase, provide oxygen tolerant variants to the transformed cells. Shuffling protocols that include versions of genes that possess desirable characteristics, such as the iron hydrogenase gene from Desulfovibrio vulgaris, which is reversibly inactivated by oxygen, are likely to generate shuffled genes with multiple desirable characteristics from different parent genes.

In one embodiment cells transformed with mutagenized nucleic acid sequences are cultured in the presence of metronidazole and are selected for the ability to produce increased amounts of hydrogen according to known methods (U.S. Pat. No. 5,871,952).

Alternatively other sensing methods are utilized. Compounds that reversibly react with hydrogen are used to synthesize films that are placed either directly on or in proximity to distinct colonies on culture plates or culture containers. The film changes a detectable characteristic in the presence of hydrogen, such as a change of color or a change from clear to opaque. In one embodiment, a substrate containing a hydrogen-dissociative catalyst metal such as tungsten trioxide is placed on or near colonies of cells and turns from transparent to blue/opaque in the presence of hydrogen (U.S. Pat. No. 6,277,589).

There are other methods, both direct and indirect, that are used to detect hydrogen, such as spectroscopic methods (U.S. Pat. No. 6,309,604). Other types of gas sensors suitable for detection of hydrogen are well known in the art.

Colonies of cells transformed with mutagenized sequences corresponding to differentially regulated genes that produce an increased amount of hydrogen under a given set of conditions than the starting strain or cells transformed with only vector and/or marker sequences are identified in this screening step. These novel strains are then utilized for the production of hydrogen

In one embodiment, the DNA construct, or substantial parts of the DNA construct, containing the mutagenized sequences is cloned, amplified, or otherwise recovered from a first strain that generates an increased amount of hydrogen The DNA construct is put through further mutagenesis protocols to generate a new library of DNA constructs used for further screening or selection of new strains that generate increased amounts of hydrogen compared to the originally identified first strain.

Nucleic acid constructs used for transforming cells may be in circular form or linear form. In addition, such constructs may be comprised of DNA or RNA. For instance, bacterial artificial chromosomes may utilized and are comprised of DNA. Alternatively, RNA vectors, such as viruses, may also be used. Viral transformation protocols for microbes are well known in the art.

In one embodiment, cells are screened for increased production of hydrogen in a high-throughput fashion after being grown on solid culture media. Colonies are identified as novel strains that produce increased amounts of hydrogen. The mutagenized sequences that impart the phenotype of the ability to produce increased amounts of hydrogen are isolated from each strain of the plurality of colonies. The isolated sequences are then put through another round of shuffling, in which the sequences are randomly cleaved, denatured, reannealed, and extended using a polymerase to generate a new library of mutagenized sequences. The sequences are then used to transform strains of the host microbe in a new round of screening or selection to generate further novel strains that produce increased amounts of hydrogen compared to the previous plurality of colonies. This process is repeated as many times as desired. High throughput methods of manipulating cells are well known in the art, and cells can be plated on solid media in densities of 9 colonies or more per square inch (Hicks, Plant Physiol 2001 December; 127(4): 1334-8).

Mating of Strains

In one embodiment, different differentially regulated genes are mutagenized and used to transform cells for screening or selection for transformants that generate an increased amount of hydrogen. Transformants that have been transformed with mutagenized nucleic acid sequences corresponding to different differentially regulated genes are then mated to each other to provide progeny containing different combinations of mutagenized nucleic acid sequences. The progeny are then screened or selected for the ability to generate an increased amount of hydrogen Screenable or selectable markers may be excised through such techniques as the Cre-lox system or FLP recombinase. Mating protocols, such as protoplast fusion, are known in the art. In addition, mating protocols for organisms such as green algae are also known (Harris, (1989) The Chlamydomonas Sourcebook. Academic Press, New York).

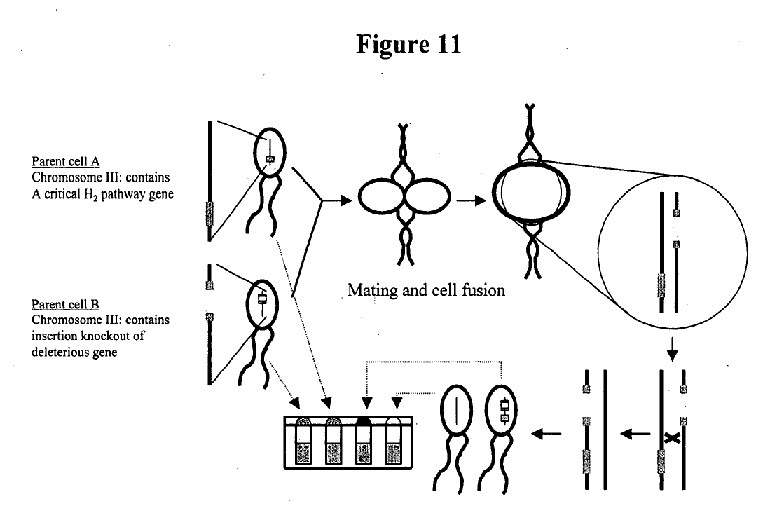

In another embodiment, cells that produce an increased amount of hydrogen due to random mutagenesis, such as chemical or insertion mutagenesis, are mated to cells that produce an increased amount of hydrogen due to mutagenized nucleic acid sequences corresponding to genes that are involved in a hydrogen production pathway. The progeny from the mating are screened or selected for the ability to generate an increased amount of hydrogen compared to any parental strain. Any strain that differs in genome sequence from a wild-type strain that produces an increased amount of hydrogen compared to the strain from which it is derived can be mated to a second strain distinct in genome sequence from the first strain that also produces an increased amount of hydrogen compared to the strain from which it is derived. Progeny from the mating are screened or selected for the ability to produce an increased amount of hydrogen compared to either parent. This type of mating, referred to as pairwise mating, is depicted in FIG. 11.

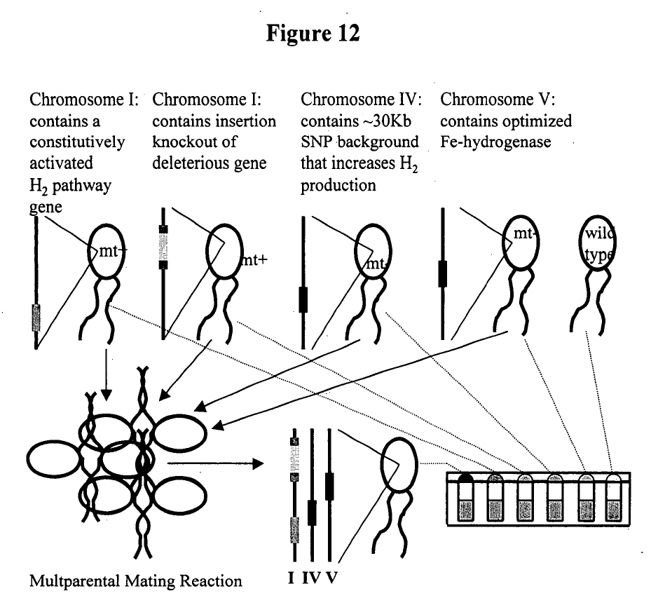

In another embodiment, three or more strains that have distinct genome sequences and produce an increased amount of hydrogen are mated to each other in a multiparental mating reaction, and the progeny are screened or selected for the ability to produce an increased amount of hydrogen compared parental strains. In green algae multiparental mating, cells are induced to undergo gametogenesis by removing nitrogen from the media. Cells mate to form zygospores. The cells are induced to germinate by adding nitrogen back to the media. The population is then induced to mate again by removing nitrogen to induce gametogenesis again, followed by adding nitrogen back to the media. The process can be repeated as many times as desired, allowing for shuffling of genomes. Because green algae are of mating type + or −, and because cells only mate with cells of the opposite mating type, at least one strain in the multiparental mating reaction must be of opposite mating type from at least one other strain in the reaction. Multiparental mating is described further in Example 3 and is depicted in FIG. 12. Multiparental mating in green algae such as Chlamydomonas can be achieved through cycling the level of nitrogen in the media and allowing the different strains to mate and produce progeny. Preferably more than one nitrogen deprivation mating cycle is performed before the cells are screened or elected for a desired phenotype. Multiparental mating allows multiple advantageous genetic alterations in the genome sequence of distinct strains to be concentrated into a single genome, allowing the individual phenotypic effect of each genetic alteration to be exerted in the presence of the other phenotypic effects of the other genetic alterations. Concentrating multiple advantageous genetic alterations therefore allows for additive or synergistic effects of multiple genetic alterations to achieved. In one embodiment, the progeny of the mating are screened for the ability to generate an increased amount of hydrogen compared to all parental strains using multiwell plates containing photoautotrophic culture media, where chemochromic films are placed over the multiwell plates. A major advantage of multiparental mating is that genetic alterations that originate in cells of the same mating type can be put into the same strain through repeated nitrogen cycling in a mating reaction. Progeny from multiparental mating reactions can be screened or selected for any desired phenotype, including hydrogen production, dissolved solid transport in or out of cells, ability to survive in certain environments such as high sunlight, low sunlight, or light of a certain wavelength, or ability to survive in environments such as high salt, low salt or brackish water, the ability to bind or decompose an environmental pollutant such as PCBs, heavy metals, dioxins, and other molecules, the ability to live on a certain food source, the ability to synthesize a desired molecule, a large number of chloroplasts per cell, and any other desired phenotype.

In another mating embodiment that can be performed as pairwise or multiparental mating, a library of C. reinhardtii strains, isolated from geographically diverse regions and containing naturally occurring single nucleotide polymorphisms (SNPs), is subjected to mating and screening or selection for a desired phenotype such as hydrogen production. The strains are subjected to the above-described mating protocols, with or without mutagenesis of the strains before or after mating. In one embodiment, the cells are transformed with an expression vector constitutively expressing an iron hydrogenase before they are mated and screened or selected for the ability to generate an increased amount of hydrogen. In one embodiment, the strains that are subjected to mating are selected from the group of strains comprising (using the strain numbers of the Chlamydomonas Genetics Center, Duke University): CC-124, CC-125, CC-1690, CC-1692, CC-407, CC-408, CC-1952, CC-2290, CC-2342, CC-2343, CC-2344, CC-2931, CC-2932, CC-2935, CC-2936, CC-2937, CC-2938, CC-2935, CC-2936, CC-2937, CC-2938, CC-3059, CC-3060, CC-3061, CC-3062, CC-3063, CC-3064, CC-3065, CC-3067, CC-3068, CC-3071, CC-3073, CC-3074, CC-3075, CC-3076, CC-3078, CC-3079, CC-3080, CC-3082, CC-3083, CC-3084, CC-3086, CC-1373 and CC-3087. These strains were isolated from geographically diverse regions and contain SNPs relative to each other's genome. These strains can also be assayed for phenotypes other than hydrogen production, such as those described in the preceding paragraph.

The multiparental mating can also be between cells other than Chlamydomonas, and the stimulus to induce gametogenesis can be other than nitrogen or other nutrient deprivation. For example, the stimulus can be the removal of light during exponential growth followed by addition of light in mating reactions with diatoms such as T. weissfloggi (Armbrust EV Appl Environ Microbiol. 1999 July; 65(7):3121-8). Alternatively, the stimulus can be addition of a compound or element such as 1 mg/liter Chromium (VI) to cells such as Scenedesmus acutus (Corradi, Ecotoxicol Environ Saf. 1995 October; 32(1):12-8; Corradi, Ecotoxicol Environ Saf. 1995 March; 30(2):106-10.).

In another embodiment, promoter sequences from a plurality of genes in the genome of an organism are used to transform cells, followed by screening or selection for a desired phenotype. For example, a plurality of 500, 1000, 1500, 2000, or more base pair promoters are amplified from the C. reinhardtii genome. The full genome sequence has been completed and can be found at http://genome.jgi-psf.org/chlrel/chlrel.home.html. The promoter sequences are connected to a selectable marker sequence and used to transform the nuclear and/or chloroplast and/or mitochondrial genome. The surviving transformants are screened or selected for a desired phenotype. Preferably, the transformants are screened for a phenotype related to a metabolic function such as the ability to produce hydrogen. Optionally, independent transformants of promoter contructs that produce an increased amount of hydrogen are mated and the progeny are screened for a further increased amount of hydrogen over any of the parents. The mating can be paiurwise or multiparental.

Methods of Producing Hydrogen

In one embodiment, cells containing mutagenized nucleic acid sequences and capable of producing an increased amount of hydrogen are cultured in a culture container with a transparent top section in an outdoor environment. Cells are grown in minimal culture media containing water, trace amounts of metals, and inorganic salts. Preferably only photoautotrophic organisms can live in the media. Atmospheric air contacts the top surface of the culture media. Nucleic acid sequences that are involved in the production of hydrogen are transcribed from constitutive, light-induced, or dark-induced promoters. Hydrogen evolved from cells is removed from the top of the culture container. During non-daylight hours, cells, for example, become dormant, metabolize molecules such as acetate to replenish substrate for digestion and hydrogen production during daylight, or produce hydrogen through a non-photosynthetic pathway. Optionally, cells are synchronized to the same phase of the cell cycle when producing hydrogen.

### EXAMPLE 1

Step 1: Sequence design: Unique sequences a-1 were searched for similarity to known sequences in the Chlamydomonas genome using the WU-Blast 2.0 program on databases of the Chlamydomonas Genome Project, located at (http://www.biology.duke.edu/chlamy_genome/blast/blast_form.html). The search produced no high scoring segment pairs. The following databases were searched: Contig Set, EST clones, S1D2 ESTs, Volvocales (non-EST), and BAC-ends (JGI). Searches were performed using the WU-blastn program using the default matrix blosum62. Gapped alignments were allowed for. The default expected threshold, filter, word length, and cutoff scores were used. The sum statistics option was used for assessing the significance of aligned pairs. Primer and chimeric oligonucleotide sequences were designed using sequences from the lhcb1 gene promoter (SEQ. ID NO 1), the 3′ untranslated region of the RBCS2 gene (SEQ. ID NO 3), and a selectable marker cassette (SEQ. ID NO 2).

Step 2: Culturing microbes under conditions not conducive and more conducive to the generation of hydrogen: Chlamydomonas reinhardtii (strain cc-124, Chlamydomonas Genetics Center, Duke University, Durham, N.C.) is cultured under conditions not conducive to the generation of hydrogen (photoheterotrophically on Tris-acetate-phosphate medium (TAP), pH 7.2 (Harris, (1989) The Chlamydomonas Sourcebook. Academic Press, New York; Melis, Plant Physiol (2000) January; 122(1):127-36). The culture is bubbled with 3% CO₂ in air, stirred gently (at approximately 400 rpm) at 25° C., under continuous illumination (approximately 300 μE m⁻²s⁻¹). The cells are grown until mid-log phase (approximately 4×10⁶ cells mL⁻¹) and then harvested by centrifugation at 2000×g for 5 minutes. The pellet is divided half mRNA is purified from one half of the pellet immediately after harvesting, as specified below, without freezing. The other half is washed 2 times in TAP-minus-sulfur and resuspended in the same medium to a final concentration of 4-5×10⁶ cells mL⁻¹ (Zhang, Planta (2002) February; 214(4):552-61; Melis, Plant Physiol (2000) January; 122(1):127-36). The cells are cultured in containers sealed from the atmosphere, under illumination (approximately 300 μE m⁻² s⁻¹), and are gently stirred at approximately 400 rpm. The containers allow gas evolved from the algae to escape into the atmosphere but do not allow atmospheric gas to enter the culture. The cells are cultured under these conditions for approximately 60 hours. The cells are then harvested by centrifugation at 2000×g for 5 minutes. RNA is purified immediately after harvesting, without freezing of the cell pellet.

Step 3: mRNA purification: mRNA is purified from both sets of cells using the Qiagen Oligotex® system (compositions of buffers OL1, ODB, and OW1 are proprietary; these buffers are purchased directly from Qiagen Inc., Valencia, Calif.). DEPC-treated water is used to make all buffers. 2-5×10⁷ cells are separated from the pellet for mRNA purification. The Oligotex® reagent is heated to 37° C. in a water bath, vortexed, and set out at room temperature. 5 mM Tris.Cl pH 7.5 is heated at 70° C. All supernatant is removed from cell pellets. 800 μL of 10 mM Tris.Cl pH 7.5, 140 mM NaCl, 5 mM KCl, 1% Nonidet P-40, 1 mM DTT, and (optionally with RNase inhibitors added), chilled at 4° C., is added and the pellet is resuspended. The suspension is incubated on ice for 5 minutes. The suspension is pelleted in a microcentrifuge tube for 2 minutes at between 300-500×g at 4° C. The supernatant is transferred to anew tube. 800 μL of room temperature 1M LiCl, 20 mM Tris.Cl pH 7.5, 2 mM EDTA, 1% SDS and 145 μL of the Oligotex® suspension are added to the supernatant, which is then vortexed. The resulting mixture is then incubated at 70° C. for 3 minutes and then at 20-30° C. for 10 minutes. The mixture is pelleted in a microcentrifuge at 14,000-18,000×g for 5 minutes. The supernatant is removed. The pellet is resuspended in 200 μL of Qiagen buffer OL1 (containing 14.3 μL β-mercaptoethanol per mL of OL1). 800 μL of Qiagen buffer ODB is added and the suspension is incubated at 70° C. for 3 minutes and room temperature for 10 minutes. The suspension is then pelleted in a microcentrifuge at maximum speed for 5 minutes. The supernatant is removed. The pellet is then resuspended in 600 μL of Qiagen buffer OW1. The suspension is then pipetted onto a large Qiagen Oligotex spin column placed inside a 2 mL microcentrifuge tube and is centrifuged for 1 minute at maximum speed. The spin column is then placed in an RNase-free 2 mL microcentrifuge tube. 600 μL of 10 mM Tris.Cl pH 7.5, 1 mM EDTA, 150 mM NaCl is added to the spin column, which is then centrifuged for 1 minute at maximum speed. The flow through is discarded and 600 μL of 10 mM Tris.Cl pH 7.5, 1 nM EDTA, 150 mM NaCl is added to the spin column, which is then centrifuged again for 1 minute at maximum speed. The spin column is then placed in a new Rnase-free 2 mL microcentrifuge tube. Approximately 200 μL of 70° C. 5 mM Tris.Cl pH 7.5 is added to the spin column. The resin is resuspended by pipetting the buffer:resin mix several times. The spin column is then centrifuged for 1 minute at maximum speed. The flow through is pipetted to a new RNase-free tube. The elution process is repeated with another 200 μL of 70° C. 5 mM Tris.Cl pH 7.5 and the flow through is added to the first flow through. The concentration and purity of the RNA is analyzed using spectrophotometric analysis.

Step 4: cDNA synthesis and in vitro transcription: Double stranded, labeled, cDNA is synthesized from the purified mRNA samples using the Invitrogen Life Technologies Superscript® Choice system (Invitrogen Inc., Carlsbad, Calif.). mRNA samples from cells cultured under conditions not conducive to the generation of hydrogen and from cells cultured under conditions more conducive to the generation of hydrogen are processed simultaneously. 4 μg of mRNA from each sample are put into RNAse-free microcentrifuge tubes, along with 100 pmol HPLC-purified primer of the sequence 5′-GGCCAGTGAATTGTAATACGACTCACTATAG GGAGGCGG-(dT)₂₄-3′. The tube is incubated at 70° C. for 10 minutes, briefly centrifuged, and placed on ice for 5 minutes. The following reagents are added: (1) 1 μL 10 mM dNTP mix; (2) 2 μL 100 mM DTT; (3) 4 μL 5× first strand cDNA buffer (proprietary composition, available from Invitrogen Inc, Carlsbad, Calif.). The reaction is then incubated at 37° C. for 2 minutes. 4 μL of 200 U/μL SuperScript® II reverse transcriptase is added to the reaction to make a final volume of 20 μL. The reaction is then incubated at 37° C. for 1 hour. The reaction is then placed on ice and the following regents are added and mixed: 91 μL of DEPC-treated water, 30 μL of 5× second strand reaction buffer (proprietary composition, available from Invitrogen Inc, Carsbad, Ca.), 3 μL of 10 mM dNTP mix, 1 μL of 10 U/μL E. coli DNA ligase, 4 μL of 10 U/μL E. coli DNA polymerase I, and 1 μL of 2 U/μL E. coli Rnase H. The reaction is incubated at 16° C. for 2 hours. 2 μL of 5 U/μL T4 DNA Polymerase is added to the reaction and it is incubated for 5 minutes at 16° C. 10 μL 0.5M EDTA is added to the reaction.

The reaction is put through a phenol:chloroform extraction using a Phase-Lock gel (optionally the reaction is put through a standard phenol:chloroform extraction). The Phase-Lock gel is pelleted in a 1.5 mL microcentrifuge tube at 12,000×g for 30 seconds. 162 μL of 25:24:1 phenol:chloroform:isoarnyl alcohol (saturated with 10 mM Tris.HCl pH 8.0, 1 mM EDTA) is added to the 162 μL reaction to a total 324 μL. The mixture is briefly vortexed, and the entire 324 μL is then added to the Phase-Lock gel tube. The tube is centrifuged at ≧12,000×g for 2 minutes. The upper aqueous layer containing the cDNAs is transferred to a new 1.5 mL tube. 0.5 volumes of 7.5 M NH₄OAc and 2.5 volumes of 100% ethanol are added to the cDNAs. The tube is vortexed and then centrifuged at ≧12,000×g for 20 minutes. The supernatant is removed and the pellet is washed with 500 μL of 80% ethanol. The tube is then centrifuged at ≧12,000×g for 5 minutes. The wash is repeated once. The pellet is then air dried and resuspended in 12 μL RNase-free water. The cDNA sample from cells cultured under conditions conducive to the generation of hydrogen is labeled as the “conducive C. rein sample.” The cDNA sample from cells cultured under conditions not conducive to the generation of hydrogen is labeled as the “nonconducive C. rein sample.” The cDNA samples are put through in vitro transcription reactions and are biotin labeled using the Enzo® BioArray® High Yield RNA Labeling Kit (available as part No. 900182 from Affymetrix Inc. Santa Clara, Calif.).

Step 5: Labeled in vitro transcript purification: Total amounts of RNA generated from the in vitro transcription reactions are determined by spectrophotometric and/or gel electrophoresis. Biotin-labeled RNA samples that originated from cells cultured under conditions not conducive to the generation of hydrogen and biotin-labeled RNA samples that originated from cells cultured under conditions more conducive to the generation of hydrogen are processed simultaneously. 600-800 μg of biotin-labeled RNA are purified on Qiagen RNeasy® midi columns. All centrifugations and reactions are performed at room temperature. For smaller or larger amounts of biotin-labeled RNA, mini or maxi columns are used, respectively, along with modified protocols according to the manufacturer. The labeled RNA is added to a tube, and is brought up to a volume of 1 mL with RNAse-free water. 4 mL of buffer RLT is added (compositions of buffers RLT, RW1, and RPE are proprietary; these buffers are purchased directly from Qiagen Inc., Valencia, Calif.) and the sample is mixed. 2.8 mL 100% ethanol and the sample is mixed. The sample is immediately applied to a Qiagen RNeasy® midi column, which is placed in a 50 mL tube, and centrifuged 5 minutes at 3,000-5,000×g. The flow through is discarded. 2.5 mL of buffer RPE is added to the column, which is then centrifuged 2 minutes at 3,000-5,000×g. The flow through is discarded. 2.5 mL of buffer RPE is again added to the column, which is then centrifuged 5 minutes at 3,000-5,000×g. The column is placed in a new 15 mL RNase-free tube. 250 μL of RNase-free water is added to the column. The column is allowed to sit for 1 minute and is then centrifuged 3 minutes at 3,000-5,000×g. Another 250 μL of RNase-free water is added to the column. The column is allowed to sit for 1 minute and is then centrifuged 3 minutes at 3,000-5,000×g. The concentration of the eluted biotin-labeled RNA is measured spectrophotometrically. If the concentration is less than 0.6 μg/μL, the biotin-labeled RNA is precipitated by adding 0.5 volumes 7.5 M NH₄OAc and 2.5 volumes 100% ethanol and resuspended in a smaller volume of RNase free water. The tube is vortexed and then placed at −20° C. for at least 1 hour. The tube is centrifuged at ≧12,000×g at 4° C. for 30 minutes. The pellet is washed twice with 500 μL of −20° C. 80% ethanol. The pellet is air dried and resuspended in 10 μL RNase-free water. The concentration of biotin-labeled RNA is adjusted to 2 μg/μL.

Step 6: Labeled in vitro transcript fragmentation:12 μL of 2 μg/μL biotin-labeled RNA is added to an RNase-free tube along with 3 μL of 5× fragmentation buffer (200 mM Tris-acetate pH 8.1, 500 mM KOAc, 150 mM MgOAc). The tube is placed at 94° C. for 35 minutes and then placed on ice. The biotin-labeled RNA is fragmented into sizes from approximately 35-200 nucleotides, and this is confirmed by gel electrophoresis using appropriate size markers.

Step 7: Microarray hybridization and differential expression profile creation: Microarray chips containing 2,761 unique C. reinhardtii sequences are obtained from the Chlamydomonas Genome Project (Duke University, Durham, N.C. http://wwv.biology.duke.edu/chlamy_genome/microarrays.html). Sequence IDs and grid locations for clones are obtained from the same source (at ftp://ftp.biology.duke.edu/pub/chlamy_genome/sequences/). Fragmented biotin labeled RNA samples are hybridized to C. reinhardtii microarrays according to Affymetrix GeneChip Expression Analysis protocols (Affymetrix Inc., Santa Clara, Calif.). Microarrays with labeled nonconductive RNA samples hybridized and microarrays with labeled conducive RNA samples hybridized are compared and analyzed for identification of differentially regulated genes. The microarray data set containing the expression data from cells cultured under conditions not conducive to the generation of hydrogen and cells cultured under conditions more conducive to the generation of hydrogen is a differential expression profile.

Step 8: Creation of probes corresponding to differentially regulated genes: Genes that exhibit greater than a 1.5-fold difference in expression between cells cultured under conditions not conducive to the generation of hydrogen and cells cultured under conditions more conducive to the generation of hydrogen are identified as differentially regulated genes. The 5 genes (referred to hereinafter as the 1H₂, 2H₂, 3H₂, 4H₂, and 5H₂ genes, and collectively as the 1-5H₂ set) that are not expressed in cells cultured under conditions not conducive to the generation of hydrogen and are upregulated most compared to other upregulated genes when cells are switched from conditions not conducive to the generation of hydrogen to conditions more conducive to the generation of hydrogen are selected for mutagenesis. Alternatively, the iron-hydrogenase gene is designated as on of the 5 genes, regardless of its expression level relative to other genes. PCR primers are designed corresponding to a 50-200 base pair segment of each gene of the 1-5H₂ set, wherein the segment chosen does not contain a specific restriction enzyme site corresponding to restriction enzymes that leave 5′ overhangs at cut sites. For example, the restriction enzymes BamHI, Hind III, and Bgl II leave 5′ overhangs after cutting double stranded DNA. The PCR primers contain the restriction enzyme sequence chosen at their 5′ end. The primers are used to amplify their corresponding fragment from each gene of the 1-5H₂ set using the conducive C. rein cDNA sample as a template. PCR products are digested with the restriction enzyme corresponding to the ends of amplified fragments. The PCR products are purified from the digested ends using agarose gel electrophoresis and electroelution from the gel fragment. The electroeluted PCR products, referred to hereinafter as the 1-5H₂ set probes, are precipitated from the electroelution buffer with 0.5 volumes of 7.5 M NH₄OAc and 2 volumes of −20° C. 100% ethanol. The 1-5H₂ set probes are pelleted at 14,000×g. The pellets are washed two times with −20° C. 70% ethanol. The pellets are dried and resuspended in water.

Step 9: Culturing microbes capable of producing hydrogen and creation of cDNA libraries: The following species of Chlamydomonas are cultured under conditions more conducive to the generation of hydrogen (available from the UTEX collection at The University of Texas at Austin, Austin, Tex.): (1) Chlamydomonas pulvinata (UTEX strain number 212, isolated from Switzerland); (2) Chlamydomonas pygmaea (UTEX strain number 2539, isolated from Prudhoe Bay, Ak.); (3) Chlamydomonas radiata (UTEX strain number 966, isolated from McMahan, Tex.); (4) Chlamydomonas rapa (UTEX strain number 1342, isolated from Danube River, Bratislava, Czechoslovakia); (5) Chlamydomonas sajao (UTEX strain number 2277, isolated from Sa Jiao, China); (6) Chlamydomonas segnis²²² (UTEX strain number 222, isolated from West Humble, Surrey, England); (7) Chlamydomonas segnis¹⁶³⁸ (UTEX strain number 1638, isolated from Dauphin Is., Ala., U.S.A.); (8) Chlamydomonas segnis¹⁹¹⁹ (UTEX strain number 1919, isolated from Delta Marsh; Manitoba, Canada); (9) Chlamydomonas smithii (UTEX strain number 1061, isolated from Santa Cruz, Calif., U.S.A.); (10) Chlamydomonas sphaeroides (UTEX strain number 221, isolated from India); (11) Chliamydomonas surtseyiensis (UTEX strain number 1796, isolated from Surtsey, Iceland); (12) Chlamydomonas ulvaensis (UTEX strain number 724, isolated from Ulva Island, Scotland); (13) Chlamydomonas zimbabwiensis (UTEX strain number 2213, isolated from Zimbabwe); (14) Chlamydomonas reinhardtii (strain cc124, Chlamydomonas Genetics Center, Duke University, Durham, N.C.). The species are cultured in TAP-minus-sulfur medium. The cells are cultured in containers sealed from the atmosphere, under illumination (approximately 300 uE mn²s⁻¹), and are gently stirred at approximately 400 rpm. The containers allow gas evolved from the algae to escape into the atmosphere but do not allow atmospheric gas to enter the culture. The cells are cultured under these conditions for approximately 60 hours. The cells are then harvested by centrifugation at 2000×g for 5 minutes. mRNA is purified immediately after harvesting, without freezing of the cell pellets. mRNA is purified from each Chlamydomonas strain as previously described using the Qiagen Oligotex® system.

cDNA libraries are made from each Chlamydomonas mRNA sample. Double stranded cDNA is synthesized from the purified mRNA samples using the Invitrogen Life Technologies Superscript® Choice system. mRNA samples from each Chlamydomonas strain are processed in parallel. 4 μL of 1 μg/μL mRNA in DEPC-treated water is added to an RNase-free centrifuge tube. 2 μL of 0.5 μg/μL oligo(dT)₁₂₋₁₈ primer and 2 μL of 50 ng/μL of random hexamer primers are added to the mRNA. The sample is heated at 70° C. for 10 minutes and immediately transferred to ice. The sample is briefly centrifuged and the following components are added: (1) 4 μL of 250 mM Tris.HCl pH 8.3, 375 mM KCl, 15 mM MgCl₂; (2) 2 μL of 100 mM DTT; (3) 1 μL of 10 mM dNTPs; (4) 1 μL 1 μCi/μL [α-³²P]dCTP. The reaction is mixed and incubated at 37° C. for 2 minutes. 4 μL of 200 U/μL of SuperScript® Reverse Transcriptase II is added to the reaction, which is mixed and incubated at 37° C. for one hour and then placed on ice. 18 μL of the reaction is placed into a new tube. The following reagents are also added: (1) 93 μL of DEPC-treated water; (2) 30 μL of 100 mM Tris.HCl pH 6.9, 450 mM KCl, 23 mM MgCl₂, 0.75 mM β-NAD⁺, 50 mM (NH₄)SO₄; (3) 3 μL 10 mM dNTT's; (4)1 μL of 10 U/μL E. coli DNA ligase; (5) 4 μL of 10 U/μL E. coli DNA Polymerase I; (6) 1 μL of 2 U/μL E. coli RNase H. The reaction is briefly vortexed, briefly centrifuged, and incubated for 2 hours at 16° C. 2 μL of 5 U/μL T4 DNA Polymerase is added and the reaction is incubated 5 minutes at 16° C. The reaction is then placed on ice and 10 μL of 0.5 M EDTA is added. 150 μL of 25:24:1 phenol:chloroform:isoamyl alcohol is added to the reaction, which is then vortexed and centrifuged at room temperature for 5 minutes at 14,000×g. 140 μL of the upper aqueous phase is transferred to a new microcentrifuge tube. 70 μL of 7.5 M NH₄OAc and 500 μL of −20° C. 100% ethanol are added to the sample. The tube is vortexed and centrifuged at room temperature for 5 minutes at 14,000×g. The supernatant is removed and the pellet is washed with 500 μL of −20° C. 70% ethanol. The tube is centrifuged at room temperature for 2 minutes at 14,000×g and the supernatant is discarded. The pellet is dried at 37° C. for 10 minutes. The pellet is resuspended in: (1) 18 μL of DEPC-treated water; (2) 10 μL of 330 mM Tris.HCl pH 7.6, 50 mM MgCl₂, 5 mM ATP; (3) 10 μL of 1 μg/μL EcoRI (Not I) adapters; (4) 7 μL of 100 mM DTT; (5) 5 μL of 1 U/μL T4 DNA ligase. The reaction is mixed and incubated for 24 hours at 16° C. The reaction is then incubated at 70° C. for 10 minutes and then placed on ice. 3 μL of 10 U/μL T4 Polynucleotide Kinase is added to the sample, which is mixed and then incubated for 0.5 hours at 37° C. The reaction is then incubated for 10 minutes at 70° C. and placed on ice. For each sample, a 1 mL pre-packed Sephacryl S-500 HR column is drained of 20% ethanol. 800 μL of 10 mM Tris.HCl pH 7.5, 0.1 mM EDTA, 25 mM NaCl is pipetted onto the top of each column. The column is allowed to drain. The wash is performed 3 more times with the same volume. 97 μL of 10 mM Tris.HCl pH 7.5, 0.1 mM EDTA, 25 mM NaCl is added to each reaction and mixed. The reaction is added to the top of the tube and drained into a first microcentrifuge tube. 100 μL of 10 mM Tris.HCl pH 7.5, 0.1 mM EDTA, 25 mM NaCl is added to the top of the column and drained into a second microcentrifuge tube. 100 μL of 10 mM Tris.HCl pH 7.5, 0.1 mM EDTA, 25 mM NaCl is added to the top of the column and each drop flowing from the bottom of the tube is collected into a new tube. The process is continued with 100 μL of 10 mM Tris.HCl pH 7.5, 0.1 mM EDTA, 25 mM NaCl being added to the top of the column until 18 drops are collected in 18 successive tubes numbered 3-20. The volume in all 20 tubes is measured. The numerical volume of each tube is added to determine the fraction of column flow through in each tube. Tubes containing volume collected after 600 μL of eluate has flowed through the column are discarded. The remaining tubes are placed in a scintillation counter and Cerenkov counts for each tube are measured. Tubes containing only background Cerenkov counts are discarded. The concentration of cDNA in each remaining fraction is determined according to the SuperScript® Choice System for cDNA Synthesis manufacturer's recommendations (Invitrogen Inc., Carlsbad, Calif., Catalog Series 18090). Fractions containing more than 0.1 ng/μL cDNA are pooled. The cDNAs are precipitated with 0.5 volumes of 7.5 M NH₄OAc and 2 volumes of −20° C. 100% ethanol. The sample is vortexed and centrifuged at room temperature for 20 minutes at 14,000×g. The pellet is washed two times with 500 μL of −20° C. 70% ethanol and then dried at 37° C. for 10 minutes. The pellet is resuspended in 20 μL 10 mM Tris.HCl pH 7.5, 0.1 mM EDTA, 25 mM NaCl. A dilution of each Chlamydomonas cDNA is made to yield 10 μL of 1 ng/μL cDNA in 10 mM Tris.HCl pH 7.5, 0.1 mM EDTA, 25 mM NaCl. All Chlamydomonas cDNA samples are processed in parallel. To each cDNA tube, the following reagents are added: (1) 4 μL of 250 mM Tris.HCl pH 7.6, 50 mM MgCl2, 5 mM ATP, 5 mM DTT, 25% (w/v) Polyethylene glycol 8000; (2) 5 μL of 10 ng/μL, EcoRI cut, dephosphorylated plasmid pcDNA3(+) (available from Invitrogen Inc., Carlsbad, Calif.); (3)1 μL of 1 U/μL T4 DNA ligase. The reaction, hereinafter referred to for each strain as the “X strain conducive cDNA library” (such as the Chlamydomonas surtseyiensis conducive cDNA library), is incubated 3 hours at room temperature and then frozen at −20° C.

Step 10: Cloning of 1-5H₂ set cDNAs: The 1-5H₂ set probes are labeled with [α-³²P]dNTPs using the Klenow DNA Polymerase fragment (available from New England Biolabs Inc., Beverly, Mass.) according to standard protocols. The conducive cDNA libraries from the fourteen Chlamydomonas strains grown in step 9 are used to transform competent E. coli cells using standard protocols. The plated E. coli cells transformed with each of the fourteen conducive cDNA libraries are used for cloning cDNAs for each of the 1-5H₂ set gene homologues from each of the fourteen conducive cDNA libraries using standard cDNA cloning methods (Maniatis et al. (1989) Molecular Cloning: A Laboratory Manual Cold Spring Harbor Laboratory). The probes used to identify each of the 1-5H₂ set gene homologues are the 1-5H₂ set probes. The identified clones are sequenced. Full length cDNAs are obtained using RACE-PCR with mRNA samples from each Chlamydomonas strain as template. A full length cDNA from each of the 1-5H₂ set gene homologues is selected for use in DNA shuffling and is referred to as the X strain Y H₂ gene (such as the Chlamydomonas pygmaea 3H₂ gene). A total of 70 cDNA sequences are obtained (a 1H₂, 2H₂, 3H₂, 4H₂, and 5H₂ gene from each of the 14 Chlamydomonas strains).

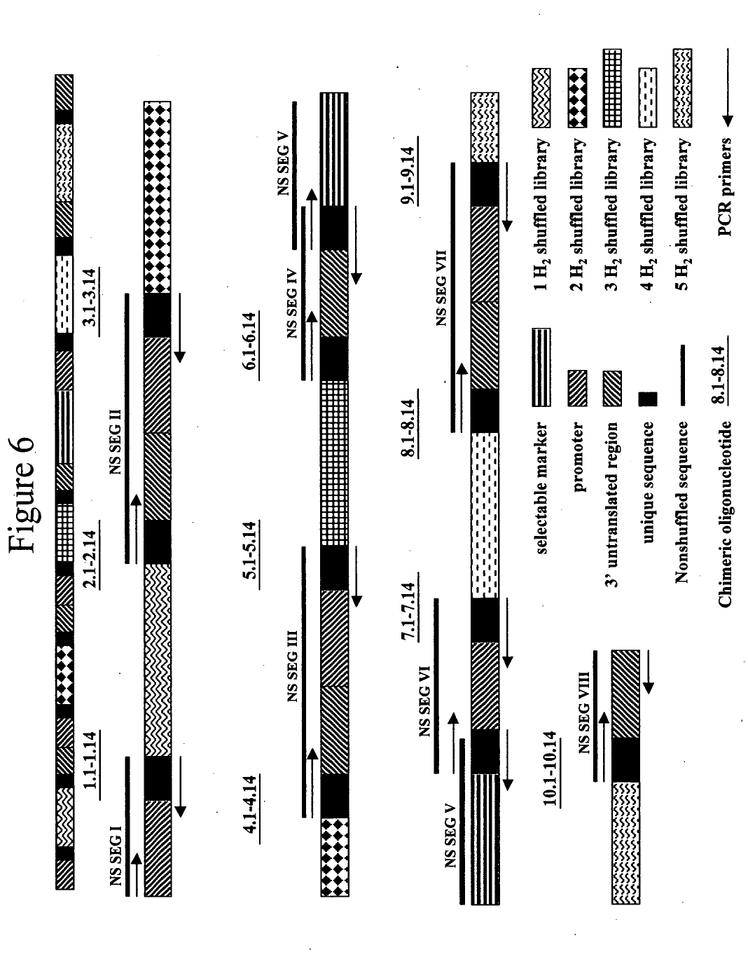

Step 11: Creation of nonshuffled DNA construct segments: Nonshuffled segments I-VIII are generated through PCR amplification using primers and templates listed in Table 1. The position of these primers relative to the sequence information they contain (not drawn to scale) is depicted in FIG. 6 by arrows. Nonshuffled segments I-VIII are gel purified, electroeluted, and precipitated. The fragments are resuspended in water.

Step 12: Shuffling of 1-5H₂ set coding regions: The coding region of each of the 70 1-5H₂ set homologue genes is amplified using the cDNA plasmid as template and primers corresponding to the N and complement of the C terminal portions of the cDNA coding sequences. PCR products corresponding to the coding regions of all 1-5H₂ set homologue genes are gel-purified, electroeluted, precipitated, and resuspended in 50 mM Tris.HCl pH 7.4, 1 MM MgCl₂. Alternatively PCR primers are removed from the reaction using the Wizard® PCR product (Promega Corp, Madison, Wis.) and the PCR products are resuspended in 50 mM Tris.HCl pH 7.4, 1 mM MgCl₂. Chimeric oligonucleotides are synthesized according to Table 2 and are resuspended in 50 mM Tris.HCl pH 7.4, 1 mM MgCl₂.

70 PCR products corresponding to the coding regions of all 1-5H₂ set homologue genes are quantified with spectrophotometry. Reactions for each of the 1-5H₂ genes are performed in parallel. Equal molar amounts of each cDNA corresponding to each of the 1-5 H₂ set homologue genes are pooled in separate tubes to obtain a total of 4 ug DNA in 100 μL 50 mM Tris.HCl pH 7.4, 1 mM MgCl₂. In other words, 0.2857 μg of cDNA from each of the 14 cDNAs corresponding to the 1 H₂ gene are added to a single tube. 0.2857 μg of cDNA from each of the 14 cDNAs corresponding to the 2H₂ gene are added to a different tube, and so on, such that each H₂ gene is shuffled in a separate reaction. DNAse I (obtained from Sigma Corp., St. Louis, Mo.) is added to each tube at a concentration of 0.0015 units of Dnase I per μl of DNA. The digestion reaction proceeds for 15 minutes at room temperature and is stopped. Digestion products from approximately 20-150 base pairs are purified from 2% low melting agarose gels, electroeluted, and precipitated. An equivalent molar amount of corresponding chimeric oligonucleotides to the original starting material for each cDNA is added to each tube. For instance, a 900 base pair 1 H₂ cDNA from one of the 14 strains corresponds to 0.481 pmol ( 1/14 of 4 μg added to DNAse I digestion reaction converted to pmol for a 900 base pair double stranded fragment). For 1H₂ cDNAs of approximately 900 base pairs, 0.481 pmol of chimeric oligonucleotides 1.1-1.14 and 0.481 pmol of chimeric oligonucleotides 2.1-2.14 are added to the purified fragmented coding regions. Chimeric oligonucleotides 3.1-3.14 and 4.1-4.14 are added to 2H₂ fragments. Chimeric oligonucleotides 5.1-5.14 and 6.1-6.14 are added to 3H₂ fragments. Chimeric oligonucleotides-7.1-7.14 and 8.1-8.14 are added to 4H₂ fragments. Chimeric oligonucleotides 9.1-9.14 and 10.1-10.14 are added to 5H₂ fragments. Chimeric oligonucleotides and 20-150 base pair cDNA fragments are resuspended in 0.2 mM of each dNTP, 2.2 mM MgCl₂, 50 mM KCl, 10 mM Tris.HCl pH 9.0, 0.1% Triton X-100, to a volume of 100 μl where the DNA concentration is approximately 20 ng/μl. 1.25 units of Taq polymerase and 1.25 units of Pfu polymerase are added. Each of the 5 tubes corresponding to cDNA fragments and chimeric oligonucleotides for genes 1-5H₂ are subjected to a themocycling program of 94° C. for 60 seconds one time, followed by 40 cycles of 94° C. for 30 seconds, 55° C. for 30 seconds, and 72° C. for 30 seconds, followed by a one time incubation of 72° C. for 5 minutes. 10 μl from each reaction is brought up to 100 μl in new PCR tubes in 0.2 mM of each dNTP, 2.2 mM MgCl₂, 50 mM KCl, 10 mM Tris.HCl pH 9.0,0.1% Triton X-100, 8 μM of primers corresponding to unique sequences and the complements of unique sequences at the ends of each cDNA fragment, and 1.25 units of Taq polymerase and 1.25 units of Pfu polymerase. Shuffled 1H₂ genes are amplified by primers corresponding to unique sequence a and the complement of unique sequence b. Shuffled 2H₂ genes are amplified by primers corresponding to unique sequence c and the complement of unique sequence d. Shuffled 3H₂ genes are amplified by primers corresponding to unique sequence e and the complement of unique sequence f. Shuffled 4H₂ genes are amplified by primers corresponding to unique sequence g and the complement of unique sequence h. Shuffled 5H₂ genes are amplified by primers corresponding to unique sequence i and the complement of unique sequence j. The amplification reactions are performed in a thermocycler for a program of 94° C. for 60 seconds one time, followed by 20 cycles of 94° C. for 30 seconds, 55° C. for 30 seconds, and 72° C. for 30 seconds, followed by a one time incubation of 72° C. for 5 minutes. PCR products, now referred to as the 1H₂ shuffled library, the 2H₂ shuffled library, etc., are gel purified, electroeluted, precipitated, and resuspended in water.

Step 13: Synthesis of test constructs: Equimolar amounts of nonshuffled segments I-VIII and 1-5H₂ shuffled libraries are added together in a new primerless PCR reaction. 1 pmol each of nonshuffled segment I, nonshuffled segment H, nonshuffled segment III, nonshuffled segment IV, nonshuffled segment V, nonshuffled segment VI, nonshuffled segment VII, nonshuffled segment VIII, 1H₂ shuffled library, 2H₂ shuffled library, 3H₂ shuffled library, 4H₂ shuffled library, and 5H₂ shuffled library are brought up to a volume of 100 μl in 0.2 mM of each dNTP, 2.2 mM MgCl₂, 50 mM KCl, 10 mM Tris.HCl pH 9.0, 0.1% Triton X-100, with 2.5 units of Pfu DNA polymerase. The reaction is subjected to a thermocycling program of 94° C. for 60 seconds one time, followed by 40 cycles of 94° C. for 30 seconds, 55° C. for 30 seconds, and 72° C. for 30 seconds, followed by a one time incubation of 72° C. for 5 minutes. Double stranded primerless PCR products, now referred to as 1-5H₂ test constructs, are separated from oligonucleotides and fragments by gel electrophoresis and products of the expected size are electroeluted, precipitated, and resuspended in sterile water.

Step 14: Transformation of cells with mutagenized nucleic acid sequences: The Chlamydomonas reinhardtii strain CC-400 (a cell wall deficient strain, Chlamydomonas Genetics Center, Duke University) is grown with shaking in TAP media (Harris, (1989) The Chlamydomonas Sourcebook. Academic Press, New York; Gorman, Proc Natl Acad Sci U S A (1965) December; 54(6):1665-9) until the cells reach a density of approximately 2×10⁶ cells/ml. The cells are pelleted at 4000×g for 5 minutes and the supernatant is removed. The cell pellet is resuspended in 7.5 ml per liter of original culture of TAP medium. The following components are added, in order, to 25 sterile tubes: 300 μl of cells, 1 μg of 1-5H₂ test construct, 100 μl of sterile-filtered 20% PEG, 300 mg of sterile glass beads (prepared according to Kindle, Meth Enzymology (1998) 297: 27-38). Each tube is vortexed 15-30 seconds at high speed. The cells are removed from the tube and spread onto plates containing phleomycin (Stevens, Mol Gen Genet (1996) April 24; 251(1):23-30). Plates are incubated in low light (approximately 5 μE m⁻²s⁻²) at 25° C. for 4-6 days in atmospheric air until colonies appear.

Step 15: Screening for increased amounts of hydrogen: Phleomycin resistant colonies are transferred to new plates containing identical culture media Colonies are plated in 96-colony grids. Replica plates are also made and stored at 15° C. in low light. The 96-colony plates, made of clear plastic, are incubated in low light (approximately 5 μE m⁻²s⁻²) at 25° C. in atmospheric air for until colonies are approximately 3 mm in diameter. Chlamydomonas reinhardtii strain CC-400 is used as a control on each 96-colony plate. After colonies have grown to the desired size, 3 mm thick filter paper is placed over the plate, covering the colonies. A chemochromic film containing tungsten trioxide is placed on top of the filter paper (Seibert). A rectangular clear plastic grid design is placed directly over the chemochromic film such that the center of each square on the grid is directly over the center of a cell colony. The plates are incubated in light (approximately 55 μE m⁻²s⁻²) at 25° C. in 5% oxygen for 12 hours. The plates are illuminated from above and below. After 12 hours, each plate is photographed from the top using a digital camera within 5 seconds of removal from the incubation chamber. The images are scanned by densitometry and are subsequently screened for dark spots on the chemochromic film that indicate the production of hydrogen. Spots that are quantitatively darker than spots directly over control colonies of nontransformed Chlamydomonas reinhardtii strain CC-400 indicate cells that generate an increased amount of hydrogen. These colonies are recovered from the test plates or the replica plates.

### EXAMPLE 2

Step 1: Sequence design: Unique sequences a-h were searched for similarity to known sequences in the Chlamydomonas genome using the WU-Blast 2.0 program on databases of the Chlamydomonas Genome Project, located at (http://www.biology.duke.edu/chlamy_genome/blast/blast_form.html). The search produced no high scoring segment pairs. The following databases were searched: Contig Set, EST clones, S1D2 ESTs, Volvocales (non-EST), and BAC-ends (JGI). Searches were performed using the WU-blastn program using the default matrix blosum62. Gapped alignments were allowed for. The default expected threshold, filter, word length, and cutoff scores were used. The sum statistics option was used for assessing the significance of aligned pairs. Primer and chimeric oligonucleotide sequences were designed using sequences from the lhcb1 gene promoter (SEQ ID 148), the 3′ untranslated region of the RBCS2 gene (SEQ ID 150), and a green fluorescent protein gene (SEQ ID 179).

Step 2: Obtaining cDNA sequences: cDNA sequences are obtained, using methods previously disclosed, for: Chlamydomonas reinhardtii ferredoxin (Genbank accession number L10349, SEQ ID NO 172); Chlamydomonas reinhardtii hydrogenase (Genbank accession number AF289201, SEQ ID NO 173); Scenedesmus obliquus hydrogenase (Genbank accession number AJ271546, SEQ ID NO 177), and Chlorella fusca hydrogenase (Genbank accession number AJ298227, SEQ ID NO 178). cDNA sequences are identified using synthetic oligonucleotides corresponding to GenBank sequences as probes.

The coding region of each of the 3 iron hydrogenase genes is amplified using the cDNA plasmid as template and primers corresponding to the N and complement of the C terminal portions of the coding regions of the cDNA sequences. PCR products corresponding to the coding regions of the 6 hydrogenase genes are gel-purified, electroeluted, precipitated and resuspended in 50 mM Tris.HCl pH 7.4, 1 mM MgCl₂. Alternatively PCR primers are removed from the reaction using the Wizard® PCR product and the PCR products are resuspended in 50 mM Tris.HCl pH 7.4, 1 mM MgCl₂. Chimeric oligonucleotides are synthesized according to Table 4 and are resuspended in 50 mM Tris.HCl pH 7.4, 1 mM MgCl₂.

Step 3: Shuffling of hydrogenase coding regions: PCR products corresponding to the coding regions of the 6 hydrogenase genes are quantified using spectrophotometry. Equal molar amounts of each PCR product are pooled to obtain a total of 4 ug DNA in 100 μL 50 mM Tris.HCl pH 7.4, 1 mM MgCl₂. DNAse I is added at a concentration of 0.15 units of Dnase I per 100 μl of reaction volume. The digestion reaction proceeds for 15 minutes at room temperature and is stopped. Digestion products from approximately 20-150 base pairs are purified from 2% low melting agarose gels, electroeluted, precipitated, and resuspended in water. 0.7123 pmol of chimeric oligonucleotides 11.1, 11.2, 11.3, 11.4, 11.5, 11.6, 12.1, 12.2, 12.3, 12.4 12.5, and 12.6 are added to each tube. Chimeric oligonucleotides and 20-150 base pair hydrogenase coding region fragments are resuspended in 0.2 mM of each dNTP, 2.2 MM MgCl₂, 50 mM KCl, 10 mM Tris.HCl pH 9.0, 0.1% Triton X-100, to a volume of 100 μl where the DNA concentration is approximately 20 ng/μl. 1.25 units of Taq polymerase and 1.25 units of Pfu polymerase are added. The reaction is subjected to a themocycling program of 94° C. for 60 seconds one time, followed by 40 cycles of 94° C. for 30 seconds, 55° C. for 30 seconds, and 72° C. for 30 seconds, followed by a one time incubation of 72° C. for 5 minutes. 10 μl from the reaction is brought up to 100 μl in new PCR tubes in 0.2 mM of each dNTP, 2.2 mM MgCl₂, 50 mM KCl, 10 mM Tris.HCl pH 9.0, 0.1% Triton X-100, 8 μM of unique sequence b and the complement of unique sequence c primers, and 1.25 units of Taq polymerase and 1.25 units of Pfu polymerase. The amplification reaction is performed in a thermocycler for a program of 94° C. for 60 seconds one time, followed by 20 cycles of 94° C. for 30 seconds, 55° C. for 30 seconds, and 72° C. for 30 seconds, followed by a one time incubation of 72° C. for 5 minutes. PCR products, now referred to as the hydrogenase shuffled library, are gel purified, electroeluted, precipitated, and resuspended in water.

Step 4: Error-prone PCR of ferredoxin: The Chlamydomonas reinhardtii ferredoxin coding region (SEQ ID NO 172) is amplified by PCR using primers corresponding to the N terminal and complement of the C terminal ends of the coding region. The coding region PCR product is then subjected to PCR using chimeric oligonucleotides 13 and 14. The PCR product, consisting of the Chlamydomonas reinhardtii ferredoxin coding region flanked by unique sequences d and e, is then subjected to error-prone PCR. The error-prone PCR is performed using unique sequence d and the complement of unique sequence e as primers at a concentration of 1 μM each, in a reaction also containing: 50 ng template (ferredoxin fragment flanked by unique sequences d and e), 20 mM Tris pH 8.4, 0.3 mM MnCl₂, 3 mM MgCl₂, 50 mM KCl, 0.01% gelatin, 0.2 mM dATP, 1 mM dCTP, 1 mM dGTP, 1 mM dTTP, 1 U AmpliTaq polymerase (Perkin Elmer, Foster City, Calif.), essentially according to the method of Leung, Technique (1989) 1, 11-15. The PCR products, now referred to as the ferredoxin library, is gel purified, electroeluted, precipitated, and resuspended in water.

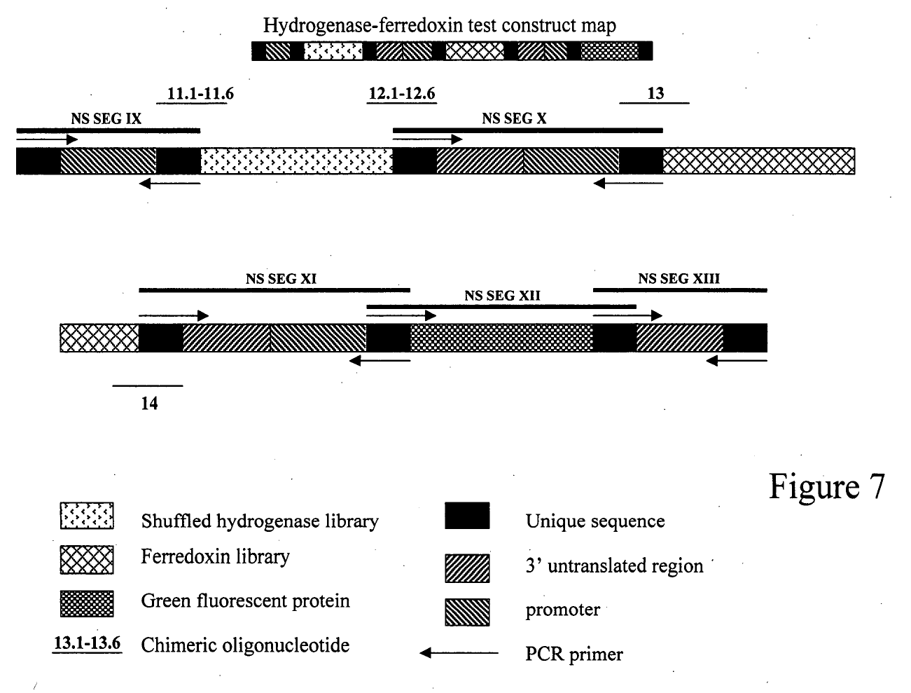

Step 5: Construction of nonshuffled segments: Nonshuffled segments IX, X, XI, XII, and XIII are generated through PCR amplification using primers and templates listed in Table 3. The position of these primers relative to the sequence information they contain (not drawn to scale) is depicted in FIG. 7 by arrows. Nonshuffled segments IX, X XI, XII, and XIII are gel purified, electroeluted, and precipitated. The fragments are resuspended in water.

Step 6: Construction of hydrogenase-ferredoxin test construct library: Equimolar amounts of nonshuffled segments IX, X, XI, XII, and XIII, the hydrogenase shuffled library and the ferredoxin library are added together in a new primerless PCR reaction. 1 pmol each of nonshuffled segments IX, X, XI, XII, and XIII, the hydrogenase shuffled library, and the ferredoxin library are brought up to a volume of 100 μl in 0.2 mM of each dNTP, 2.2 mM MgCl₂, 50 mM KCl, 10 mM Tris.HCl pH 9.0, 0.1% Triton X-100, with 2.5 units of Pfu DNA polymerase. The reaction is subjected to a themocycling program of 94° C. for 60 seconds one time, followed by 40 cycles of 94° C. for 30 seconds, 55° C. for 30 seconds, and 72° C. for 30 seconds, followed by a one time incubation of 72° C. for 5 minutes. Double stranded primerless PCR products, now referred to as hydrogenase-ferredoxin test construct library, are separated from oligonucleotides and fragments by gel electrophoresis and products of the expected size are electroeluted, precipitated, and resuspended in sterile water.

Step 7: Transformation of cells: The Chlamydomonas reinhardtii strain cc-400 is grown with shaking in TAP media (Harris, (1989) The Chlamydomonas Sourcebook. Academic Press, New York; Gorman, Proc Natl Acad Sci USA (1965) December; 54(6): 1665-9) until the cells reach a density of approximately 2×10⁶ cells/ml. The cells are pelleted at 4000×g for 5 minutes and the supernatant is removed. The cell pellet is resuspended in 7.5 ml per liter of original culture of TAP medium. The following components are added, in order, to 25 sterile tubes: 300 μl of cells, 1 μg of hydrogenase-ferredoxin test construct, 100 μl of sterile-filtered 20% PEG, 300 mg of sterile glass beads (prepared according to Kindle, Meth Enzymology (1998) 297: 27-38). Each tube is vortexed 15-30 seconds at high speed. The cells are removed from the tube and are cultured in TAP media under continuous illumination (approximately 55 μE m⁻²s⁻²) at 25° C. for 12 hours.

Step 8: Screening cells for generation of hydrogen: Cells in media are illuminated with 395 nm light and monitored for emission at 525 nm using fluorescence-activated cell sorting (Bloodgood et al. Exp Cell Res 1987 December; 173(2):572-85; Hegemann). Colonies exhibiting 525 nm GFP emission are recovered from the sorting protocol and are plated in 96 colony grids on solid media Replica plates are also made and stored at 15° C. in low light. The 96-colony plates, made of clear plastic, are incubated in low light (approximately 5 μE m⁻²s⁻²) at 25° C. in atmospheric air until colonies are approximately 3 mm in diameter. Chlamydomonas reinhardtii strain cc-400 is used as a control on each 96-colony plate. After colonies have grown to the desired size, 3 mm thick filter paper is placed over the plate, covering the colonies. A chemochromic film containing tungsten trioxide is placed on top of the filter paper (Seibert). A rectangular clear plastic grid design is placed directly over the chemochromic film such that the center of each square on the grid is directly over the center of a cell colony. The plates are incubated in light (approximately 55 μE m⁻²s⁻²) at 25° C. in atmospheric air for 12 hours. The plates are illuminated from above and below. After 12 hours, each plate is photographed from the top using a digital camera within 5 seconds of removal from the incubation chamber. The images are scanned by densitometry and are subsequently screened for dark spots on the chemochromic film that indicate the production of hydrogen. Spots that are quantitatively darker than spots directly over control colonies of nontransformed Chlamydomonas reinhardtii strain cc-400 indicate cells that generate an increased amount of hydrogen. These colonies are recovered from the test plates or the replica plates.

Step 9: Isolation and further mutagenesis of hydrogenase-ferredoxin test constructs that cause increased production of hydrogen: Total DNA is isolated from the 5% of all transformant colonies exhibiting the highest level of hydrogen production. Hydrogenase-ferredoxin test constructs are recovered from the DNA by PCR using primers corresponding to unique sequence a and the complement of unique sequence h PCR products are gel purified, electroeluted, precipitated, and resuspended in water.

The hydrogenase-ferredoxin test constructs are quantified using spectrophotometry. Equimolar amounts of each recovered test construct are added to a total of 4 μg of test construct and are diluted to 100 μL to yield a reaction tube containing 50 mM Tris.HCl pH 7.4, 1 mM MgCl₂. DNAse I is added at a concentration of 0.15 units of Dnase I per 100 μl of reaction volume. The digestion reaction proceeds for 15 minutes at room temperature. Digestion products from approximately 20-150 base pairs are purified from 2% low melting agarose gels, electroeluted, precipitated, and resuspended in 0.2 mM of each dNTP, 2.2 mM MgCl₂, 50 mM KCl, 10 mM Tris.HCl pH 9.0, 0.1% Triton X-100, to a volume of 100 μl where the DNA concentration is approximately 20 ng/μl. 1.25 units of Taq polymerase and 1.25 units of Pfu polymerase are added. The reaction is subjected to a themocycling program of 94° C. for 60 seconds one time, followed by 40 cycles of 94° C. for 30 seconds, 55° C. for 30 seconds, and 72° C. for 30 seconds, followed by a one time incubation of 72° C. for 5 minutes. 10 μl from the reaction is brought up to 100 μl in new PCR tubes in 0.2 mM of each dNTP, 2.2 mM MgCl₂, 50 mM KCl, 10 mM Tris.HCl pH 9.0, 0.1% Triton X-100, 8 μM of unique sequence a and the complement of unique sequence h primers, 1.25 units of Taq polymerase and 1.25 units of Pfu polymerase. The amplification reaction is performed in a thermocycler for with a program of 94° C. for 60 seconds one time, followed by 20 cycles of 94° C. for 30 seconds, 55° C. for 30 seconds, and 72° C. for 30 seconds, followed by a one time incubation of 72° C. for 5 minutes. PCR products, now referred to as the hydrogenase-ferredoxin secondary test constructs, are gel purified, electroeluted, precipitated, and resuspended in sterile water.

Step 10: Transformation of cells: The Chlamydomonas reinhardtii strain cc-400 is grown with shaking in TAP media (Harris, (1989) The Chlamydomonas Sourcebook. Academic Press, New York; Gorman, Proc Natl Acad Sci USA (1965) December; 54(6):1665-9) until the cells reach a density of approximately 2×10⁶ cells/ml. The cells are pelleted at 4000×g for 5 minutes and the supernatant is removed. The cell pellet is resuspended in 7.5 ml per liter of original culture of TAP medium. The following components are added, in order, to 25 sterile tubes: 300 μl of cells, 1 μg of hydrogenase-ferredoxin secondary test construct, 100 μl of sterile-filtered 20% PEG, 300 mg of sterile glass beads (prepared according to Kindle, Meth Enzymology (1998) 297: 27-38). Each tube is vortexed 15-30 seconds at high speed. The cells are removed from the tube and are cultured in TAP media under continuous illumination (approximately 55 μE m⁻²s⁻²) at 25° C. for 12 hours.

Step 11: Screening cells for generation of hydrogen: Cells in media are illuminated with 395 nm light and monitored for emission at 525 nm using fluorescence-activated cell sorting (Bloodgood et al. Exp Cell Res 1987 December; 173(2):572-85; Hegemann). Colonies exhibiting 525 nm GFP emission are recovered from the sorting protocol and are plated in 96-colony grids on solid media Replica plates are also made and stored at 15° C. in low light. The 96-colony plates, made of clear plastic, are incubated in low light (approximately 5 μE m⁻²s⁻²) at 25° C. in atmospheric air until colonies are approximately 3 mm in diameter. Chlamydomonas reinhardtii strain cc-400 is used as a control on each 96-colony plate. After colonies have grown to the desired size, 3 mm thick filter paper is placed over the plate, covering the colonies. A chemochromic film containing tungsten trioxide is placed on top of the filter paper (Seibert). A rectangular clear plastic grid design is placed directly over the chemochromic film such that the center of each square on the grid is directly over the center of a cell colony. The plates are incubated in light (approximately 55 μE m⁻²s⁻²) at 25° C. in atmospheric air for 12 hours. The plates are illuminated from above and below. After 12 hours, each plate is photographed from the top using a digital camera within 5 seconds of removal from the incubation chamber. The images are scanned by densitometry and are subsequently screened for dark spots on the chemochromic film that indicate the production of hydrogen. Spots that are quantitatively darker than spots directly over control colonies of nontransformed Chlamydomonas reinhardtii strain cc-400 indicate cells that generate an increased amount of hydrogen. These colonies are recovered and are used for hydrogen production and/or further development.

### EXAMPLE 3

Multiparental Mating Protocol

1. Place cells from 3 or more strains of algae capable of mating to each other such as Chliaydomonas reinhardtii together in the same tube, where at least one strain is of a different mating type than at least one other strain. For example, place approximately the same number of cells of the following strains into the tube: CC-124, CC-125, CC-1690, CC-1692, CC-407, CC-408, CC-1952, CC-2290, CC-2342, CC-2343, CC-2344, CC-2931, CC-2932, CC-2935, CC-2936, CC-2937, CC-2938, CC-2935, CC-2936, CC-2937, CC-2938, CC-3059, CC-3060, CC-3061, CC-3062, CC-3063, CC-3064, CC-3065, CC-3067, CC-3068, CC-3071, CC-3073, CC-3074, CC-3075, CC-3076, CC-3078, CC-3079, CC-3080, CC-3082, CC-3083, CC-3084, CC-3086, CC-1373 and CC-3087.

2. Suspend the cells nitrogen free medium, such as Sueoka's medium without NH₄Cl.

3. Incubate in light, for 12 hours, or for 1 day, or 2 days, or 3 days, or 4 days, or for 5, 6, 7, 8, 9, 10, or more days, or for fractions of the aforementioned numbers of days.

Add nitrogen (such as NH₄Cl) to media or move cells into nitrogen containing media and incubate in light, for 12 hours, or for 1 day, or 2 days, or 3 days, or 4 days, or for 5, 6, 7, 8, 9, 10, or more days, or for fractions of the aforementioned numbers of days.

5. Collect cells and change media back to nitrogen free and incubate in light for 12 hours, or for 1 day, or 2 days, or 3 days, or 4 days, or for 5, 6, 7, 8, 9, 10, or more days, or for fractions of the aforementioned numbers of days.

6. Repeat steps 4-5 as any times as desired.

7. Plate mating reaction on solid media (or optionally sort cells individually with a cell sorter) and pick colonies.

8. Array strains from colonies into multiwell plates containing liquid culture media.

9. Screen or select for a desired phenotype.

10. Identify 3 or more novel strains from step 9 that have the desired phenotype.

11. Repeat steps 1-9 as many times as desired.

To make 1 liter of Sueoka's high salt media*:

| | |
|---|---|
| Phosphate Buffer | 50 mls |
| Beijerinck's stock | 50 mls |
| Hutner's trace elements (see TAP) | 1 ml |
| Sodium acetate | 2.0 g (1.2 g if anhydrous) |

Phosphate Buffer

| | |
|---|---|
| Component | For 1 liter |
| K₂HPO₄ | 28.8 g |
| KH₂PO₄ | 14.4 g |

Beijerinck's stock

| | |
|---|---|
| Component | for 1 liter |
| NH₄Cl | 10 g |
| MgSO₄.7H₂O | 0.4 g |
| CaCl₂.2H₂O | 0.2 g |

*Media for inducing gametogenesis can be made by withholding NH₄Cl from the Beijerinck's stock

### EXAMPLE 4

Gene Reassembly

The process of chimeric gene assembly is depicted in FIGS. 13-14. Sections of the active site region that are both highly conserved and correspond to the gas channel were identified using structural data, as shown in FIG. 9. In step 1 of FIG. 13, a library of approximately 110 unique Iron hydrogenase amino acid sequences was aligned using sequence manipulation software (DS Gene 1.5, Accelyrys Inc., San Diego, Calif.). The key in FIG. 15 shows the identity of amino acids from step 1 and codons from steps 2-9. In step 2, peptide sequences of conserved gas channel segments were reverse-translated into single stranded oligonucleotide sequences using C. reinhardtii most preferred codons from FIG. 10. All bars in step 1 correspond to amino acids of aligned iron hydrogenases. All bars in steps 2-9 correspond to codons that encode the amino acids from the bars of step 1. Each bar in steps 2-9 therefore depicts a codon triplet of oligonucleotide sequence. In step 3, three codons encoding amino acids that flank each side of the conserved gas channel segments were re-written to encode the corresponding C. reinhardtii amino acids in those flanking positions. Each oligonucleotide of step 3 therefore encodes (from left to right) three C. reinhardtii codons that flank the N-terminal side of a gas channel segment, followed by codons corresponding to a non-C. reinhardtii gas channel segment, followed by three C. reinhardtii codons that flank the C-terminal side of the gas channel segment. Even though these oligonucleotides encode different sequences from the C. reinhardtii Iron hydrogenase, the combination of recoding and the substitution of 3 flanking codons on either side of the gas channel segment generates enough nucleotide similarity that these oligonucleotides anneal to a complementary strand encoding the recoded, wild-type C. reinhardtii Iron hydrogenase. In step 4, the entire set of recoded oligonucleotides is mixed and annealed to single stranded “scaffold” DNA molecules that encode the wild type C. reinhardtii Iron hydrogenase protein in recoded form. Recoding the wild type C. reinhardtii iron-hydrogenase to make the scaffold achieves maximum sequence identity between the scaffold and the recoded oligonucleotides because the wild type C. reinhardtii Iron hydrogenase gene does not contain only the most highly preferred codons. Oligonucleotides corresponding to wild type C. reinhardtii gas channel segments with single residue substitutions designed to narrow the gas channel can also be mixed into in the annealing reaction. The single stranded scaffold molecule is generated by isolating the gene from a plasmid grown in a methylating host cell, followed by denaturation and separation of the strands by HPLC or other standard procedures, as described for example in U.S. Pat. No. 6,361,974. None of the primers anneal to partially overlapping sites on the C. reinhardtii strand. No exonuclease treatment is needed to “clip” strands partially displaced by annealing of other oligonucleotide. In step 5 of FIG. 14, different combinations of diverse gas channel segments anneal to each full length complementary strand. Each oligonucleotide has at least 9 perfect base pairs on both ends, ensuring sufficient annealing despite internal mismatches due to sequence variation of the gas channel segments. Addition of DNA Polymerase in step 6 extends the annealed oligonucleotides, creating a combinatorial library of double stranded hybrid Iron hydrogenase molecules with numerous mismatches at “context” residue positions. Preferably the DNA Polymerase is exonuclease-deficient to prevent it from degrading parts of annealed primers in its path as it extends between annealed primers. In step 7, the methylated strands are digested using a methylation-sensitive endonuclease, as described for example in U.S. Pat. No. 6,361,974. An alternative method for separating the scaffold strands from the library strands is to use a biotinylated C-terminal primer and separate the library strands using immobilized streptavidin. In steps 8-9, an N and C terminal C. reinhardtii primers and DNA Polymerase are added to the library of novel Iron hydrogenase molecules for a single round of amplification. The result is a library of double stranded Iron hydrogenase sequences that have random combinations of functional gas channel segments but C. reinhardtii framework/hinge regions. The library is be cloned into C. reinhardtii cells and assayed for catalytic activity in the presence of O₂. Library members identified as active in the presence of O₂ are sequenced and a new library is made using the above method and oligonucleotides designed to anneal to a representative single stranded Iron hydrogenase identified from the first library. The screening process on the second library is performed in the presence of an additional amount of oxygen compared to the first round. This gene reassembly procdure can be used to mutagenize any nucleic acid sequence.

TABLE 1

| | | 5′ primer | | | |
|---|---|---|---|---|---|
| Product | 5′ primer | sequence | 3′ primer | 3′ primer sequence | Template |
| Nonshuffled | First 24 | 5′ gcagttgggtca | Complement | 5′ gctaagatggcc | SEQ ID NO 148 |
| segment I | nucleotides | ggggctggcgac 3′ | of unique | ataaggataactac | |
| | of promoter | | sequence a- | ggattaacgaaatg | |
| | fragment of | | complement | agtctcgcccgcggc 3′ | |
| | the lhcb1 | | of last 25 base | | |
| | gene | | pairs of the | | |
| | | | promoter | | |
| | | | fragment of | | |
| | | | the lhcb1 gene | | |
| Nonshuffled | Unique | 5′ cgtgcatcgattaa | Complement | 5′ cttagtcatacttg | SEQ ID NO 151 |
| segment II | sequence b- | cagcttctggacctga | of unique | gacgtacgacgttta | |
| | first 25 | ccgacgtcgaccca | sequence c- | ataacgaaatgagt | |
| | nucleotides | ctctagaggat 3′ | complement | ctcgcccgcggc 3′ | |
| | of 3′ UTR | | of last 25 base | | |
| | from | | pairs of the | | |
| | RBCS2 | | promoter | | |
| | gene | | fragment of | | |
| | | | the lhcb1 gene | | |
| Nonshuffled | Unique | 5′ aatctgatac | Complement | 5′ agttacgatttact | SEQ ID NO 151 |
| segment III | sequence d- | atgctattca | of unique | agtcgagtagacat | |
| | first 25 | gatcttacaa | sequence e- | tttaacgaaatgag | |
| | nucleotides | ccgacgtcgaccca | complement | tctcgcccgcggc 3′ | |
| | of 3′ UTR | ctctagaggat 3′ | of last 25 base | | |
| | from | | pairs of the | | |
| | RBCS2 | | promoter | | |
| | gene | | fragment of | | |
| | | | the lhcb1 gene | | |
| Nonshuffled | Unique | 5′ atctgtaata | Complement | 5′ cgaatcctcgttag | SEQ ID NO 150 |
| segment IV | sequence f- | atctagtcga | of unique | taactattccgactac | |
| | first 25 | ggcattcaag | sequence k- | caaatacgccca | |
| | nucleotides | ccgacgtcgaccca | complement | gcccgcccatgg 3′ | |
| | of 3′ UTR | ctctagaggat 3′ | of last 24 | | |
| | from | | nucleotides of | | |
| | RBCS2 | | 3′ UTR from | | |
| | gene | | RBCS2 gene | | |
| Nonshuffled | Unique | 5′ gtagtcggaatagtt | Complement | 5′ agttacgatttactag | SEQ ID NO 149 |
| segment V | sequence k- | actaacgaggattcg | of unique | tcgagtagacattt | |
| | First 25 | gccagaaggag | sequence l- | ggtaccgggccc | |
| | nucleotides | cgcagccaaaccag 3′ | complement | cccctcgagtta 3′ | |
| | of the ble | | of last 25 | | |
| | selectable | | nucleotides of | | |
| | marker | | the ble | | |
| | cassette | | selectable | | |
| | | | marker | | |
| | | | cassette | | |
| Nonshuffled | Unique | 5′ aaatgtctactcgac | Complement | 5′ tcacacgattg | SEQ ID NO 148 |
| segment VI | sequence l- | tagtaaatcgtaact | of unique | ttaacgatttaag | |
| | first 24 | gcagttgggtca | sequence g- | ccagtttaacgaaat | |
| | nucleotides | ggggctggcgac 3′ | complement | gagtctcgcccgcggc 3′ | |
| | of promoter | | of last 25 | | |
| | fragment of | | nucleotides of | | |
| | the lhcb1 | | promoter | | |
| | gene | | fragment of | | |
| | | | the lhcb1 gene | | |
| Nonshuffled | Unique | 5′ gatttaacat | Complement | 5′ ttgtcaccagga | SEQ ID NO 151 |
| segment VII | sequence h- | aactgtcgat | of unique | ttacgattgtcaagc | |
| | first 25 | taccgtgcga | sequence i- | atataacgaaatga | |
| | nucleotides | ccgacgtcgaccca | complement | gtctcgcccgcggc 3′ | |
| | of 3′ UTR | ctctagaggat 3′ | of last 25 | | |
| | from | | nucleotides of | | |
| | RBCS2 | | promoter | | |
| | gene | | fragment of | | |
| | | | the lhcb1 gene | | |
| Nonshuffled | Unique | 5′ taacaagaat | Complement | 5′ caaatacgccca | SEQ ID NO 150 |
| segment VIII | sequence j- | ctggctaatc | of last 24 | gcccgcccatgg 3′ | |
| | first 25 | aatcgatgca | nucleotides of | | |
| | nucleotides | ccgacgtcgaccca | 3′ UTR from | | |
| | of 3′ UTR | ctctagaggat 3′ | RBCS2 gene | | |
| | from | | | | |
| | RBCS2 | | | | |
| | gene | | | | |

Table 2 Key to nomenclature: Chimeric oligonucleotides are designed according to sequences derived from the 5′ and 3′ ends of the 70 cDNAs of the 1-5H₂ set. All portions of chimeric oligonucleotides corresponding to the 5′ end of a cDNA start with a start codon. For instance, the oligonucleotide 1.1 from Table 1 has a sequence of 5′ atccgtagttatccttatggccatcttagc-atg[cpul1h2]₂₇3′. This oligonucleotide's first 30 nucleotides, reading from 5′ to 3′, encode unique sequence a (SEQ ID NO 152). Nucleotides 31-33 encode a start codon (atg). After the start codon the sequence is from the 5′ end of the Chlamydomonas pulvinata 1H₂ gene coding sequence, beginning after the start codon. Sequence listed in italics corresponds to the portion of the description written in italics. All portions of chimeric oligonucleotides corresponding to the 3′ end of a cDNA end with a stop codon. For instance, the oligonucleotide 2.1 from Table 1 has a sequence of 5′ [cpul1h2]₂₇taa-cgtgcatcgattaacagcttctggacctga 3′. This oligonucleotide's first 27 nucleotides, reading from 5′ to 3′, encode the last 27 nucleotides of the Chlamydomonas pulvinata 1 H₂ gene coding sequence, followed by a stop codon. After the stop codon the sequence is unique sequence b (SEQ ID NO 153).

TABLE 2

| | | | |
|---|---|---|---|
| Oligo | | | |
| # | 5′ end corresponding to: | 3′ end corresponding to: | Sequence |

| | | | | | |
|---|---|---|---|---|---|
| 1.1 | Unique sequence a | (SEQ ID | First 30 bp of 5′ end of | | 5′ atccgtagttatccttatggccat |
| | | NO 152) | Chlamydomonas pulvinata 1 | | cttagc- |
| | | | H₂ gene coding sequence | | atg[cpul1h2]₂₇ 3′ |
| 1.2 | Unique sequence a | (SEQ ID | First 30 bp of 5′ end of | | 5′ atccgtagttatccttatggccat |
| | | NO 152) | Chlamydomonas pygmaea 1 | | cttagc- |
| | | | H₂ gene coding sequence | | atg[cpyg1h2]₂₇ 3′ |
| 1.3 | Unique sequence a | (SEQ ID | First 30 bp of 5′ end of | | 5′ atccgtagttatccttatggccat |
| | | NO 152) | Chlamydomonas radiata 1 H₂ | | cttagc- |
| | | | gene coding sequence | | atg[crad1h2]₂₇ 3′ |
| 1.4 | Unique sequence a | (SEQ ID | First 30 bp of 5′ end of | | 5′ atccgtagttatccttatggccat |
| | | NO 152) | Chlamydomonas rapa 1 H₂ | | cttagc- |
| | | | gene coding sequence | | atg[crap1h2]₂₇ 3′ |
| 1.5 | Unique sequence a | (SEQ ID | First 30 bp of 5′ end of | | 5′ atccgtagttatccttatggccat |
| | | NO 152) | Chlamydomonas sajao 1 H₂ | | cttagc- |
| | | | gene coding sequence | | arg[csaj1h2]₂₇ 3′ |
| 1.6 | Unique sequence a | (SEQ ID | First 30 bp of 5′ end of | | 5′ atccgtagttatccttatggccat |
| | | NO 152) | Chlamydomonas segnis²²² 1 | | cttagc- |
| | | | H₂ gene coding sequence | | atg[cseg²²²1h2]₂₇ 3′ |
| 1.7 | Unique sequence a | (SEQ ID | First 30 bp of 5′ end of | | 5′ atccgtagttatccttatggccat |
| | | NO 152) | Chlamydomonas segnis¹⁶³⁸ 1 | | cttagc- |
| | | | H₂ gene coding sequence | | atg[cseg¹⁶³⁸1h2]₂₇ 3′ |
| 1.8 | Unique sequence a | (SEQ ID | First 30 bp of 5′ end of | | 5′ atccgtagttatccttatggccat |
| | | NO 152) | Chlamydomonas segnis¹⁹¹⁹ 1 | | cttagc- |
| | | | H₂ gene coding sequence | | atg[cseg¹⁹¹⁹1h2]₂₇ 3′ |
| 1.9 | Unique sequence a | (SEQ ID | First 30 bp of 5′ end of | | 5′ atccgtagttatccttatggccat |
| | | NO 152) | Chlamydomonas smithii 1 H₂ | | cttagc- |
| | | | gene coding sequence | | atg[csmi1h2]₂₇ 3′ |
| 1.10 | Unique sequence a | (SEQ ID | First 30 bp of 5′ end of | | 5′ atccgtagttatccttatggccat |
| | | NO 152) | Chlamydomonas sphaeroides | | cttagc- |
| | | | H₂ gene coding sequence | | atg[csph1h2]₂₇ 3′ |
| 1.11 | Unique sequence a | (SEQ ID | First 30 bp of 5′ end of | | 5′ atccgtagttatccttatggccat |
| | | NO 152) | Chlamydomonas surtseyiensis | | cttagc- |
| | | | H₂ gene coding sequence | | atg[csur1h2]₂₇ 3′ |
| 1.12 | Unique sequence a | (SEQ ID | First 30 bp of 5′ end of | | 5′ atccgtagttatccttatggccat |
| | | NO 152) | Chlamydomonas ulvaensis 1 | | cttagc- |
| | | | H₂ gene coding sequence | | atg[culv1h2]₂₇ 3′ |
| 1.13 | Unique sequence a | (SEQ ID | First 30 bp of 5′ end of | | 5′ atccgtagttatccttatggccat |
| | | NO 152) | Chlamydomonas | | cttagc- |
| | | | zimbabwiensis 1 H₂ gene | | atg[czim1h2]₂₇ 3′ |
| | | | coding sequence | | |
| 1.14 | Unique sequence a | (SEQ ID | First 30 bp of 5′ end of | | 5′ atccgtagttatccttatggccat |
| | | NO 152) | Chlamydomonas reinhardtii 1 | | cttagc- |
| | | | H₂ gene coding sequence | | atg[crei1h2]₂₇ 3′ |
| 2.1 | Last 30 bp of 3′ end of | | Unique sequence b | (SEQ ID | 5′ [cpul1h2]₃₀-cgtgcatcga |
| | Chlamydomonas | | | NO 153) | ttaacagcttctggacctga 3′ |
| | pulvinata 1 H₂ gene | | | | |
| | coding sequence | | | | |
| 2.2 | Last 30 bp of 3′ end of | | Unique sequence b | (SEQ ID | 5′ [cpyg1h2]₂₇taa- |
| | Chlamydomonas | | | NO 153) | cgtgcatcgattaacagcttctggacc |
| | pygmaea 1 H₂ gene | | | | tga 3′ |
| | coding sequence | | | | |
| 2.3 | Last 30 bp of 3′ end of | | Unique sequence b | (SEQ ID | 5′ [crad1h2]₂₇taa- |
| | Chlamydomonas radiata | | | NO 153) | cgtgcatcgattaacagcttctggacc |
| | 1 H₂ gene coding | | | | tga 3′ |
| | sequence | | | | |
| 2.4 | Last 30 bp of 3′ end of | | Unique sequence b | (SEQ ID | 5′ [crap1h2]₂₇taa- |
| | Chlamydomonas rapa 1 | | | NO 153) | cgtgcatcgattaacagcttctggacc |
| | H₂ gene coding sequence | | | | tga 3′ |
| 2.5 | Last 30 bp of 3′ end of | | Unique sequence b | (SEQ ID | 5′ [csaj1h2]₂₇taa- |
| | Chlamydomonas sajao 1 | | | NO 153) | cgtgcatcgattaacagcttctggacc |
| | H₂ gene coding sequence | | | tga 3′ | |
| 2.6 | Last 30 bp of 3′ end of | | Unique sequence b | (SEQ ID | 5′ [cseg²²²1h2]₂₇taa- |
| | Chlamydomonas | | | NO 153) | cgtgcatcgattaacagcttctggacc |
| | segnis²²² 1 H₂ gene | | | | tga 3′ |
| | coding sequence | | | | |
| 2.7 | Last 30 bp of 3′ end of | | Unique sequence b | (SEQ ID | 5′ [cseg¹⁶³⁸1h2]₂₇taa- |
| | Chlamydomonas | | | NO 153) | cgtgcatcgattaacagcttctggacc |
| | segnis¹⁶³⁸ 1 H₂ gene | | | | tga 3′ |
| | coding sequence | | | | |
| 2.8 | Last 30 bp of 3′ end of | | Unique sequence b | (SEQ ID | 5′ [cseg¹⁹¹⁹1h2]₂₇taa- |
| | Chlamydomonas | | | NO 153) | cgtgcatcgattaacagcttctggacc |
| | segnis¹⁹¹⁹ 1 H₂ gene | | | | tga 3′ |
| | coding sequence | | | | |
| 2.9 | Last 30 bp of 3′ end of | | Unique sequence b | (SEQ ID | 5′ [csmi1h2]₂₇taa- |
| | Chlamydomonas smithii | | | NO 153) | cgtgcatcgattaacagcttctggacc |
| | 1 H₂ gene coding | | | | tga 3′ |
| | sequence | | | | |
| 2.10 | Last 30 bp of 3′ end of | | Unique sequence b | (SEQ ID | 5′ [csph1h2]₂₇taa- |
| | Chlamydomonas | | | NO 153) | cgtgcatcgattaacagcttctggacc |
| | sphaeroides 1 H₂ gene | | | | tga 3′ |
| | coding sequence | | | | |
| 2.11 | Last 30 bp of 3′ end of | | Unique sequence b | (SEQ ID | 5′ [csur1h2]₂₇taa- |
| | Chlamydomonas | | | NO 153) | cgtgcatcgattaacagcttctggacc |
| | surtseyiensis 1 H₂ gene | | | | tga 3′ |
| | coding sequence | | | | |
| 2.12 | Last 30 bp of 3′ end of | | Unique sequence b | (SEQ ID | 5′ [culv1h2]₂₇taa- |
| | Chlamydomonas | | | NO 153) | cgtgcatcgattaacagcttctggacc |
| | ulvaensis 1 H₂ gene | | | | tga 3′ |
| | coding sequence | | | | |
| 2.13 | Last 30 bp of 3′ end of | | Unique sequence b | (SEQ ID | 5′ [czmi1h2]₂₇taa- |
| | Chlamydomonas | | | NO 153) | cgtgcatcgattaacagcttctggacc |
| | zimbabwiensis 1 H₂ gene | | | | tga 3′ |
| | coding sequence | | | | |
| 2.14 | Last 30 bp of 3′ end of | | Unique sequence b | (SEQ ID | 5′ [crei1h2]₂₇taa- |
| | Chlamydomonas | | | NO 153) | cgtgcatcgattaacagcttctggacc |
| | reinhardtii 1 H₂ gene | | | | tga 3′ |
| | coding sequence | | | | |
| 3.1 | Unique sequence c | (SEQ ID | First 30 bp of 5′ end of | | 5′ ttaaacgtcgtacgtccaagtata |
| | | NO 154) | Chlamydomonas pulvinata 2 | | actaag- |
| | | | H₂ gene coding sequence | | atg[cpul1h2]₂₇ 3′ |
| 3.2 | Unique sequence c | (SEQ ID | First 30 bp of 5′ end of | | 5′ ttaaacgtcgtacgtccaagtata |
| | | NO 154) | Chlamydomonas pygmaea 2 | | actaag- |
| | | | H₂ gene coding sequence | | atg[cpyg1h2]₂₇ 3′ |
| 3.3 | Unique sequence c | (SEQ ID | First 30 bp of 5′ end of | | 5′ ttaaacgtcgtacgtccaagtata |
| | | NO 154) | Chlamydomonas radiata 2 H₂ | | actaag- |
| | | | gene coding sequence | | atg[crad1h2]₂₇ 3′ |
| 3.4 | Unique sequence c | (SEQ ID | First 30 bp of 5′ end of | | 5′ ttaaacgtcgtacgtccaagtata |
| | | NO 154) | Chlamydomonas rapa 2 H₂ | | actaag- |
| | | | gene coding sequence | | atg[crap1h2]₂₇ 3′ |
| 3.5 | Unique sequence c | (SEQ ID | First 30 bp of 5′ end of | | 5′ ttaaacgtcgtacgtccaagtata |
| | | NO 154) | Chlamydomonas sajao 2 H₂ | | actaag- |
| | | | gene coding sequence | | atg[csaj1h2]₂₇ 3′ |
| 3.6 | Unique sequence c | (SEQ ID | First 30 bp of 5′ end of | | 5′ ttaaacgtcgtacgtccaagtata |
| | | NO 154) | Chlamydomonas segnis²²² 2 | | actaag- |
| | | | H₂ gene coding sequence | | atg[cseg²²²1h2]₂₇ 3′ |
| 3.7 | Unique sequence c | (SEQ ID | First 30 bp of 5′ end of | | 5′ ttaaacgtcgtacgtccaagtata |
| | | NO 154) | Chlamydomonas segnis¹⁶³⁸ 2 | | actaag- |
| | | | H₂ gene coding sequence | | atg[cseg¹⁶³⁸1h2]₂₇ 3′ |
| 3.8 | Unique sequence c | (SEQ ID | First 30 bp of 5′ end of | | 5′ ttaaacgtcgtacgtccaagtata |
| | | NO 154) | Chlamydomonas segnis¹⁹¹⁹ 2 | | actaag- |
| | | | H₂ gene coding sequence | | atg[cseg¹⁹¹⁹1h2]₂₇ 3′ |
| 3.9 | Unique sequence c | (SEQ ID | First 30 bp of 5′ end of | | 5′ ttaaacgtcgtacgtccaagtata |
| | | NO 154) | Chlamydomonas smithii 2 H₂ | | actaag- |
| | | | gene coding sequence | | atg[csmi1h2]₂₇ 3′ |
| 3.10 | Unique sequence c | (SEQ ID | First 30 bp of 5′ end of | | 5′ ttaaacgtcgtacgtccaagtata |
| | | NO 154) | Chlamydomonas sphaeroides | | actaag- |
| | | | 2 H₂ gene coding sequence | | atg[csph1h2]₂₇ 3′ |
| 3.11 | Unique sequence c | (SEQ ID | First 30 bp of 5′ end of | | 5′ ttaaacgtcgtacgtccaagtata |
| | | NO 154) | Chlamydomonas surtseyiensis | | actaag- |
| | | | 2 H₂ gene coding sequence | | atg[csur1h2]₂₇ 3′ |
| 3.12 | Unique sequence c | (SEQ ID | First 30 bp of 5′ end of | | 5′ ttaaacgtcgtacgtccaagtata |
| | | NO 154) | Chlamydomonas ulvaensis 2 | | actaag- |
| | | | H₂ gene coding sequence | | atg[culv1h2]₂₇ 3′ |
| 3.13 | Unique sequence c | (SEQ ID | First 30 bp of 5′ end of | | 5′ ttaaacgtcgtacgtccaagtata |
| | | NO 154) | Chlamydomonas | | actaag- |
| | | | zimbabwiensis 2 H₂ gene | | atg[czim1h2]₂₇ 3′ |
| | | | coding sequence | | |
| 3.14 | Unique sequence c | (SEQ ID | First 30 bp of 5′ end of | | 5′ ttaaacgtcgtacgtccaagtata |
| | | NO 154) | Chlamydomonas reinhardtii 2 | | actaag- |
| | | | H₂ gene coding sequence | | atg[crei1h2]₂₇ 3′ |
| 4.1 | Last 30 bp of 3′ end of | | Unique sequence d | (SEQ ID | 5′ [cpul2h2]₂₇taa- |
| | Chlamydomonas | | | NO 155) | aatctgatacatgctattcagatctta |
| | pulvinata 1 H₂ gene | | | | caa 3′ |
| | coding sequence | | | | |
| 4.2 | Last 30 bp of 3′ end of | | Unique sequence d | (SEQ ID | 5′ [cpyg2h2]₂₇taa- |
| | Chlamydomonas | | | NO 155) | aatctgatacatgctattcagatctta |
| | pygmaea 2 H₂ gene | | | | caa 3′ |
| | coding sequence | | | | |
| 4.3 | Last 30 bp of 3′ end of | | Unique sequence d | (SEQ ID | 5′ [crad2h2]₂₇taa- |
| | Chlamydomonas radiata | | | NO 155) | aatctgatacatgctattcagatctta |
| | 2 H₂ gene coding | | | | caa 3′ |
| | sequence | | | | |
| 4.4 | Last 30 bp of 3′ end of | | Unique sequence d | (SEQ ID | 5′ [crap2h2]₂₇taa- |
| | Chlamydomonas rapa 2 | | | NO 155) | aatctgatacatgctattcagatctta |
| | H₂ gene coding sequence | | | | caa 3′ |
| 4.5 | Last 30 bp of 3′ end of | | Unique sequence d | (SEQ ID | 5′ [csaj2h2]₂₇taa- |
| | Chlamydomonas sajao 2 | | | NO 155) | aatctgatacatgctattcagatctta |
| | H₂ gene coding sequence | | | | caa 3′ |
| 4.6 | Last 30 bp of 3′ end of | | Unique sequence d | (SEQ ID | 5′ [cseg²²²2h2]₂₇taa- |
| | Chlamydomonas | | | NO 155) | aatctgatacatgctattcagatctta |
| | segnis²²² 2 H₂ gene | | | | caa 3′ |
| | coding sequence | | | | |
| 4.7 | Last 30 bp of 3′ end of | | Unique sequence d | (SEQ ID | 5′ [cseg¹⁶³⁸2h2]₂₇taa- |
| | Chlamydomonas | | | NO 155) | aatctgatacatgctattcagatctta |
| | segnis¹⁶³⁸ 2 H₂ gene | | | | caa 3′ |
| | coding sequence | | | | |
| 4.8 | Last 30 bp of 3′ end of | | Unique sequence d | (SEQ ID | 5′ [cseg¹⁹¹⁹2h2]₂₇taa- |
| | Chlamydomonas | | | NO 155) | aatctgatacatgctattcagatctta |
| | segnis¹⁹¹⁹ 2 H₂ gene | | | | caa 3′ |
| | coding sequence | | | | |
| 4.9 | Last 30 bp of 3′ end of | | Unique sequence d | (SEQ ID | 5′ [csmi2h2]₂₇taa- |
| | Chlamydomonas smithii | | | NO 155) | aatctgatacatgctattcagatctta |
| | 2 H₂ gene coding | | | | caa 3′ |
| | sequence | | | | |
| 4.10 | Last 30 bp of 3′ end of | | Unique sequence d | (SEQ ID | 5′ [csph2h2]₂₇taa- |
| | Chlamydomonas | | | NO 155) | aatctgatacatgctattcagatctta |
| | sphaeroides 2 H₂ gene | | | | caa 3′ |
| | coding sequence | | | | |
| 4.11 | Last 30 bp of 3′ end of | | Unique sequence d | (SEQ ID | 5′ [csur2h2]₂₇taa- |
| | Chlamydomonas | | | NO 155) | aatctgatacatgctattcagatctta |
| | surtseyiensis 2 H₂ gene | | | | caa 3′ |
| | coding sequence | | | | |
| 4.12 | Last 30 bp of 3′ end of | | Unique sequence d | (SEQ ID | 5′ [culv2h2]₂₇taa- |
| | Chlamydomonas | | | NO 155) | aatctgatacatgctattcagatctta |
| | ulvaensis 2 H₂ gene | | | | caa 3′ |
| | coding sequence | | | | |
| 4.13 | Last 30 bp of 3′ end of | | Unique sequence d | (SEQ ID | 5′ [czim2h2]₂₇taa- |
| | Chlamydomonas | | | NO 155) | aatctgatacatgctattcagatctta |
| | zimbabwiensis 2 H₂ gene | | | | caa 3′ |
| | coding sequence | | | | |
| 4.14 | Last 30 bp of 3′ end of | | Unique sequence d | (SEQ ID | 5′ [crei2h2]₂₇taa- |
| | Chlamydomonas | | | NO 155) | aatctgatacatgctattcagatctta |
| | reinhardtii 2 H₂ gene | | | | caa 3′ |
| | coding sequence | | | | |
| 5.1 | Unique sequence e | (SEQ ID | First 30 bp of 5′ end of | | 5′ aaatgtctactcgactagtaaatc |
| | | NO 156) | Chlamydomonas pulvinata 3 | | gtaact- |
| | | | H₂ gene coding sequence | | atg[cpul3h2]₂₇ 3′ |
| 5.2 | Unique sequence e | (SEQ ID | First 30 bp of 5′ end of | | 5′ aaatgtctactcgactagtaaatc |
| | | NO 156) | Chlamydomonas pygmaea 3 | | gtaact- |
| | | | H₂ gene coding sequence | | atg[cpyg3h2]₂₇ 3′ |
| 5.3 | Unique sequence e | (SEQ ID | First 30 bp of 5′ end of | | 5′ aaatgtctactcgactagtaaatc |
| | | NO 156) | Chlamydomonas radiata 3 H₂ | | gtaact- |
| | | | gene coding sequence | | atg[crad3h2]₂₇ 3′ |
| 5.4 | Unique sequence e | (SEQ ID | First 30 bp of 5′ end of | | 5′ aaatgtctactcgactagtaaatc |
| | | NO 156) | Chlamydomonas rapa 3 H₂ | | gtaact- |
| | | | gene coding sequence | | atg[crap3h2]₂₇ 3′ |
| 5.5 | Unique sequence e | (SEQ ID | First 30 bp of 5′ end of | | 5′ aaatgtctactcgactagtaaatc |
| | | NO 156) | Chlamydomonas sajao 3 H₂ | | gtaact- |
| | | | gene coding sequence | | atg[csaj3h2]₂₇ 3′ |
| 5.6 | Unique sequence e | (SEQ ID | First 30 bp of 5′ end of | | 5′ aaatgtctactcgactagtaaatc |
| | | NO 156) | Chlamydomonas segnis²²² 3 | | gtaact- |
| | | | H₂ gene coding sequence | | atg[cseg²²²3h2]₂₇ 3′ |
| 5.7 | Unique sequence e | (SEQ ID | First 30 bp of 5′ end of | | 5′ aaatgtctactcgactagtaaatc |
| | | NO 156) | Chlamydomonas segnis¹⁶³⁸ 3 | | gtaact- |
| | | | H₂ gene coding sequence | | atg[cseg¹⁶³⁸3h2]₂₇ 3′ |
| 5.8 | Unique sequence e | (SEQ ID | First 30 bp of 5′ end of | | 5′ aaatgtctactcgactagtaaatc |
| | | NO 156) | Chlamydomonas segnis¹⁹¹⁹ 3 | | gtaact- |
| | | | H₂ gene coding sequence | | atg[cseg¹⁹¹⁹3h2]₂₇ 3′ |
| 5.9 | Unique sequence e | (SEQ ID | First 30 bp of 5′ end of | | 5′ aaatgtctactcgactagtaaatc |
| | | NO 156) | Chlamydomonas smithii 3 H₂ | | gtaact- |
| | | | gene coding sequence | | atg[csmi3h2]₂₇ 3′ |
| 5.10 | Unique sequence e | (SEQ ID | First 30 bp of 5′ end of | | 5′ aaatgtctactcgactagtaaatc |
| | | NO 156) | Chlamydomonas sphaeroides | | gtaact- |
| | | | 3 H₂ gene coding sequence | | atg[csph3h2]₂₇ 3′ |
| 5.11 | Unique sequence e | (SEQ ID | First 30 bp of 5′ end of | | 5′ aaatgtctactcgactagtaaatc |
| | | NO 156) | Chlamydomonas surtseyiensis | | gtaact- |
| | | | 3 H₂ gene coding sequence | | atg[csur3h2]₂₇ 3′ |
| 5.12 | Unique sequence e | (SEQ ID | First 30 bp of 5′ end of | | 5′ aaatgtctactcgactagtaaatc |
| | | NO 156) | Chlamydomonas ulvaensis 3 | | gtaact- |
| | | | H₂ gene coding sequence | | atg[culv3h2]₂₇ 3′ |
| 5.13 | Unique sequence e | (SEQ ID | First 30 bp of 5′ end of | | 5′ aaatgtctactcgactagtaaatc |
| | | NO 156) | Chlamydomonas | | gtaact- |
| | | | zimbabwiensis 3 H₂ gene | | atg[czim3h2]₂₇ 3′ |
| | | | coding sequence | | |
| 5.14 | Unique sequence e | (SEQ ID | First 30 bp of 5′ end of | | 5′ aaatgtctactcgactagtaaatc |
| | | NO 156) | Chlamydomonas reinhardtii 3 | | gtaact- |
| | | | H₂ gene coding sequence | | atg[crei3h2]₂₇ 3′ |
| 6.1 | Last 30 bp of 5′ end of | | Unique sequence f | (SEQ ID | 5′ [cpul3h2]₂₇taa- |
| | Chlamydomonas | | | NO 157) | atctgtaataatctagtcgaggcattc |
| | pulvinata 3 H₂ gene | | | | aag 3′ |
| | coding sequence | | | | |
| 6.2 | Last 30 bp of 3′ end of | | Unique sequence f | (SEQ ID | 5′ [cpyg3h2]₂₇taa- |
| | Chlamydomonas | | | NO 157 | atctgtaataatctagtcgaggcattc |
| | pygmaea 3 H₂ gene | | | | aag 3′ |
| | coding sequence | | | | |
| 6.3 | Last 30 bp of 3′ end of | | Unique sequence f | (SEQ ID | 5′ [crad3h2]₂₇taa- |
| | Chlamydomonas radiata | | | NO 157 | atctgtaataatctagtcgaggcattc |
| | 3 H₂ gene coding | | | | aag 3′ |
| | sequence | | | | |
| 6.4 | Last 30 bp of 3′ end of | | Unique sequence f | (SEQ ID | 5′ [crap3h2]₂₇taa- |
| | Chlamydomonas rapa 3 | | | NO 157 | atctgtaataatctagtcgaggcattc |
| | H₂ gene coding sequence | | | | aag 3′ |
| 6.5 | Last 30 bp of 3′ end of | | Unique sequence f | (SEQ ID | 5′ [csaj3h2]₂₇taa- |
| | Chlamydomonas sajao 3 | | | NO 157 | atctgtaataatctagtcgaggcattc |
| | H₂ gene coding sequence | | | | aag 3′ |
| 6.6 | Last 30 bp of 3′ end of | | Unique sequence f | (SEQ ID | 5′ [cseg²²²3h2]₂₇taa- |
| | Chlamydomonas | | | NO 157 | atctgtaataatctagtcgaggcattc |
| | segnis²²² 3 H₂ gene | | | | aag 3′ |
| | coding sequence | | | | |
| 6.7 | Last 30 bp of 3′ end of | | Unique sequence f | (SEQ ID | 5′ [cseg¹⁶³⁸3h2]₂₇taa- |
| | Chlamydomonas | | | NO 157 | atctgtaataatctagtcgaggcattc |
| | segnis¹⁶³⁸ 3 H₂ gene | | | | aag 3′ |
| | coding sequence | | | | |
| 6.8 | Last 30 bp of 3′ end of | | Unique sequence f | (SEQ ID | 5′ [cseg¹⁹¹⁹3h2]₂₇taa- |
| | Chlamydomonas | | | NO 157 | atctgtaataatctagtcgaggcattc |
| | segnis¹⁹¹⁹ 3 H₂ gene | | | | aag 3′ |
| | coding sequence | | | | |
| 6.9 | Last 30 bp of 3′ end of | | Unique sequence f | (SEQ ID | 5′ [csmi3h2]₂₇taa- |
| | Chlamydomonas smithii | | | NO 157 | atctgtaataatctagtcgaggcattc |
| | 3 H₂ gene coding | | | | aag 3′ |
| | sequence | | | | |
| 6.10 | Last 30 bp of 3′ end of | | Unique sequence f | (SEQ ID | 5′ [csph3h2]₂₇taa- |
| | Chlamydomonas | | | NO 157 | atctgtaataatctagtcgaggcattc |
| | sphaeroides 3 H₂ gene | | | | aag 3′ |
| | coding sequence | | | | |
| 6.11 | Last 30 bp of 3′ end of | | Unique sequence f | (SEQ ID | 5′ [csur3h2]₂₇taa- |
| | Chlamydomonas | | | NO 157 | atctgtaataatctagtcgaggcattc |
| | surtseyiensis 3 H₂ gene | | | | aag 3′ |
| | coding sequence | | | | |
| 6.12 | Last 30 bp of 3′ end of | | Unique sequence f | (SEQ ID | 5′ [culv3h2]₂₇taa- |
| | Chlamydomonas | | | NO 157 | atctgtaataatctagtcgaggcattc |
| | ulvaensis 3 H₂ gene | | | | aag 3′ |
| | coding sequence | | | | |
| 6.13 | Last 30 bp of 3′ end of | | Unique sequence f | (SEQ ID | 5′ [czim3h2]₂₇taa- |
| | Chlamydomonas | | | NO 157 | atctgtaataatctagtcgaggcattc |
| | zimbabwiensis 3 H₂ gene | | | | aag 3′ |
| | coding sequence | | | | |
| 6.14 | Last 30 bp of 3′ end of | | Unique sequence f | (SEQ ID | 5′ [crei3h2]₂₇taa- |
| | Chlamydomonas | | | NO 157 | atctgtaataatctagtcgaggcattc |
| | reinhardtii 3 H₂ gene | | | | aag 3′ |
| | coding sequence | | | | |
| 7.1 | Unique sequence g | (SEQ ID | First 30 bp of 5′ end of | | 5′ aactggcttaaatcgttaacaatc |
| | | NO 158) | Chlamydomonas pulvinata 4 | | gtgtga- |
| | | | H₂ gene coding sequence | | atg[cpul4h2]₂₇ 3′ |
| 7.2 | Unique sequence g | (SEQ ID | First 30 bp of 5′ end of | | 5′ aactggcttaaatcgttaacaatc |
| | | NO 158) | Chlamydomonas pygmaea 4 | | gtgtga- |
| | | | H₂ gene coding sequence | | atg[cpyg4h2]₂₇ 3′ |
| 7.3 | Unique sequence g | (SEQ ID | First 30 bp of 5′ end of | | 5′ aactggcttaaatcgttaacaatc |
| | | NO 158) | Chlamydomonas radiata 4 H₂ | | gtgtga- |
| | | | gene coding sequence | | atg[crad4h2]₂₇ 3′ |
| 7.4 | Unique sequence g | (SEQ ID | First 30 bp of 5′ end of | | 5′ aactggcttaaatcgttaacaatc |
| | | NO 158) | Chlamydomonas rapa 4 H₂ | | gtgtga- |
| | | | gene coding sequence | | atg[crap4h2]₂₇ 3′ |
| 7.5 | Unique sequence g | (SEQ ID | First 30 bp of 5′ end of | | 5′ aactggcttaaatcgttaacaatc |
| | | NO 158) | Chlamydomonas sajao 4 H₂ | | gtgtga- |
| | | | gene coding sequence | | atg[csaj4h2]₂₇ 3′ |
| 7.6 | Unique sequence g | (SEQ ID | First 30 bp of 5′ end of | | 5′ aactggcttaaatcgttaacaatc |
| | | NO 158) | Chlamydomonas segnis²²² 4 | | gtgtga- |
| | | | H₂ gene coding sequence | | atg[cseg²²²4h2]₂₇ 3′ |
| 7.7 | Unique sequence g | (SEQ ID | First 30 bp of 5′ end of | | 5′ aactggcttaaatcgttaacaatc |
| | | NO 158) | Chlamydomonas segnis¹⁶³⁸ 4 | | gtgtga- |
| | | | H₂ gene coding sequence | | atg[cseg¹⁶³⁸4h2]₂₇ 3′ |
| 7.8 | Unique sequence g | (SEQ ID | First 30 bp of 5′ end of | | 5′ aactggcttaaatcgttaacaatc |
| | | NO 158) | Chlamydomonas segnis¹⁹¹⁹ 4 | | gtgtga- |
| | | | H₂ gene coding sequence | | atg[cseg¹⁹¹⁹4h2]₂₇ 3′ |
| 7.9 | Unique sequence g | (SEQ ID | First 30 bp of 5′ end of | | 5′ aactggcttaaatcgttaacaatc |
| | | NO 158) | Chlamydomonas smithii 4 H₂ | | gtgtga- |
| | | | gene coding sequence | | atg[csmi4h2]₂₇ 3′ |
| 7.10 | Unique sequence g | (SEQ ID | First 30 bp of 5′ end of | | 5′ aactggcttaaatcgttaacaatc |
| | | NO 158) | Chlamydomonas sphaeroides | | gtgtga- |
| | | | 4 H₂ gene coding sequence | | atg[csph4h2]₂₇ 3′ |
| 7.11 | Unique sequence g | (SEQ ID | First 30 bp of 5′ end of | | 5′ aactggcttaaatcgttaacaatc |
| | | NO 158) | Chlamydomonas surtseyiensis | | gtgtga- |
| | | | 4 H₂ gene coding sequence | | atg[csur4h2]₂₇ 3′ |
| 7.12 | Unique sequence g | (SEQ ID | First 30 bp of 5′ end of | | 5′ aactggcttaaatcgttaacaatc |
| | | NO 158) | Chlamydomonas ulvaensis 4 | | gtgtga- |
| | | | H₂ gene coding sequence | | atg[culv4h2]₂₇ 3′ |
| 7.13 | Unique sequence g | (SEQ ID | First 30 bp of 5′ end of | | 5′ aactggcttaaatcgttaacaatc |
| | | NO 158) | Chlamydomonas | | gtgtga- |
| | | | zimbabwiensis 4 H₂ gene | | atg[czim4h2]₂₇ 3′ |
| | | | coding sequence | | |
| 7.14 | Unique sequence g | (SEQ ID | First 30 bp of 5′ end of | | 5′ aactggcttaaatcgttaacaatc |
| | | NO 158) | Chlamydomonas reinhardtii 4 | | gtgtga- |
| | | | H₂ gene coding sequence | | atg[crei4h2]₂₇ 3′ |
| 8.1 | Last 30 bp of 5′ end of | | Unique sequence h | (SEQ ID | 5′ [cpul4h2]₂₇taa- |
| | Chlamydomonas | | | NO 159) | gatttaacataactgtcgattaccgtg |
| | pulvinata 4 H₂ gene | | | | cga 3′ |
| | coding sequence | | | | |
| 8.2 | Last 30 bp of 3′ end of | | Unique sequence h | (SEQ ID | 5′ [cpyg4h2]₂₇taa- |
| | Chlamydomonas | | | NO 159) | gatttaacataactgtcgattaccgtg |
| | pygmaea 4 H₂ gene | | | | cga 3′ |
| | coding sequence | | | | |
| 8.3 | Last 30 bp of 3′ end of | | Unique sequence h | (SEQ ID | 5′ [crad4h2]₂₇taa- |
| | Chlamydomonas radiata | | | NO 159) | gatttaacataactgtcgattaccgtg |
| | 4 H₂ gene coding | | | | cga 3′ |
| | sequence | | | | |
| 8.4 | Last 30 bp of 3′ end of | | Unique sequence h | (SEQ ID | 5′ [crap4h2]₂₇taa- |
| | Chlamydomonas rapa 4 | | | NO 159) | gatttaacataactgtcgattaccgtg |
| | H₂ gene coding sequence | | | | cga 3′ |
| 8.5 | Last 30 bp of 3′ end of | | Unique sequence h | (SEQ ID | 5′ [csaj4h2]₂₇taa- |
| | Chlamydomonas sajao 4 | | | NO 159) | gatttaacataactgtcgattaccgtg |
| | H₂ gene coding sequence | | | | cga 3′ |
| 8.6 | Last 30 bp of 3′ end of | | Unique sequence h | (SEQ ID | 5′ [cseg₂₂₂4h2]₂₇taa- |
| | Chlamydomonas | | | NO 159) | gatttaacataactgtcgattaccgtg |
| | segnis²²² 4 H₂ gene | | | | cga 3′ |
| | coding sequence | | | | |
| 8.7 | Last 30 bp of 3′ end of | | Unique sequence h | (SEQ ID | 5′ [cseg₁₆₃₈4h2]₂₇taa- |
| | Chlamydomonas | | | NO 159) | gatttaacataactgtcgattaccgtg |
| | segnis¹⁶³⁸ 4 H₂ gene | | | | cga 3′ |
| | coding sequence | | | | |
| 8.8 | Last 30 bp of 3′ end of | | Unique sequence h | (SEQ ID | 5′ [cseg₁₉₁₉4h2]₂₇taa- |
| | Chlamydomonas | | | NO 159) | gatttaacataactgtcgattaccgtg |
| | segnis¹⁹¹⁹ 4 H₂ gene | | | | cga 3′ |
| | coding sequence | | | | |
| 8.9 | Last 30 bp of 3′ end of | | Unique sequence h | (SEQ ID | 5′ [csmi4h2]₂₇taa- |
| | Chlamydomonas smithii | | | NO 159) | gatttaacataactgtcgattaccgtg |
| | 4 H₂ gene coding | | | | cga 3′ |
| | sequence | | | | |
| 8.10 | Last 30 bp of 3′ end of | | Unique sequence h | (SEQ ID | 5′ [csph4h2]₂₇taa- |
| | Chlamydomonas | | | NO 159) | gatttaacataactgtcgattaccgtg |
| | sphaeroides 4 H₂ gene | | | | cga 3′ |
| | coding sequence | | | | |
| 8.11 | Last 30 bp of 3′ end of | | Unique sequence h | (SEQ ID | 5′ [csur4h2]₂₇taa- |
| | Chlamydomonas | | | NO 159) | gatttaacataactgtcgattaccgtg |
| | surtseyiensis 4 H₂ gene | | | | cga 3′ |
| | coding sequence | | | | |
| 8.12 | Last 30 bp of 3′ end of | | Unique sequence h | (SEQ ID | 5′ [culv4h2]₂₇taa- |
| | Chlamydomonas | | | NO 159) | gatttaacataactgtcgattaccgtg |
| | ulvaensis 4 H₂ gene | | | | cga 3′ |
| | coding sequence | | | | |
| 8.13 | Last 30 bp of 3′ end of | | Unique sequence h | (SEQ ID | 5′ [czim4h2]₂₇taa- |
| | Chlamydomonas | | | NO 159) | gatttaacataactgtcgattaccgtg |
| | zimbabwiensis 4 H₂ gene | | | | cga 3′ |
| | coding sequence | | | | |
| 8.14 | Last 30 bp of 3′ end of | | Unique sequence h | (SEQ ID | 5′ [crei4h2]₂₇taa- |
| | Chlamydomonas | | | NO 159) | gatttaacataactgtcgattaccgtg |
| | reinhardtii 4 H₂ gene | | | | cga 3′ |
| | coding sequence | | | | |
| 9.1 | Unique sequence i | (SEQ ID | First 30 bp of 5′ end of | | 5′ tatgcttgacaatcgtaatcctgg |
| | | NO 160) | Chlamydomonas pulvinata 5 | | tgacaa- |
| | | | H₂ gene coding sequence | | atg[cpul5h2]₂₇ 3′ |
| 9.2 | Unique sequence i | (SEQ ID | First 30 bp of 5′ end of | | 5′ tatgcttgacaatcgtaatcctgg |
| | | NO 160) | Chlamydomonas pygmaea 5 | | tgacaa- |
| | | | H₂ gene coding sequence | | atg[cpyg5h2]₂₇ 3′ |
| 9.3 | Unique sequence i | (SEQ ID | First 30 bp of 5′ end of | | 5′ tatgcttgacaatcgtaatcctgg |
| | | NO 160) | Chlamydomonas radiata 5 H₂ | | tgacaa- |
| | | | gene coding sequence | | atg[crad5h2]₂₇ 3′ |
| 9.4 | Unique sequence i | (SEQ ID | First 30 bp of 5′ end of | | 5′ tatgcttgacaatcgtaatcctgg |
| | | NO 160) | Chlamydomonas rapa 5 H₂ | | tgacaa- |
| | | | gene coding sequence | | atg[crap5h2]₂₇ 3′ |
| 9.5 | Unique sequence i | (SEQ ID | First 30 bp of 5′ end of | | 5′ tatgcttgacaatcgtaatcctgg |
| | | NO 160) | Chlamydomonas sajao 5 H₂ | | tgacaa- |
| | | | gene coding sequence | | atg[csaj5h2]₂₇ 3′ |
| 9.6 | Unique sequence i | (SEQ ID | First 30 bp of 5′ end of | | 5′ tatgcttgacaatcgtaatcctgg |
| | | NO 160) | Chlamydomonas segnis²²² 5 | | tgacaa- |
| | | | H₂ gene coding sequence | | atg[cseg²²²5h2]₂₇ 3′ |
| 9.7 | Unique sequence i | (SEQ ID | First 30 bp of 5′ end of | | 5′ tatgcttgacaatcgtaatcctgg |
| | | NO 160) | Chlamydomonas segnis¹⁶³⁸ 5 | | tgacaa- |
| | | | H₂ gene coding sequence | | atg[cseg¹⁶³⁸5h2]₂₇ 3′ |
| 9.8 | Unique sequence i | (SEQ ID | First 30 bp of 5′ end of | | 5′ tatgcttgacaatcgtaatcctgg |
| | | NO 160) | Chlamydomonas segnis¹⁹¹⁹ 5 | | tgacaa- |
| | | | H₂ gene coding sequence | | atg[cseg¹⁹¹⁹5h2]₂₇ 3′ |
| 9.9 | Unique sequence i | (SEQ ID | First 30 bp of 5′ end of | | 5′ tatgcttgacaatcgtaatcctgg |
| | | NO 160) | Chlamydomonas smithii 5 H₂ | | tgacaa- |
| | | | gene coding sequence | | atg[csmi5h2]₂₇ 3′ |
| 9.10 | Unique sequence i | (SEQ ID | First 30 bp of 5′ end of | | 5′ tatgcttgacaatcgtaatcctgg |
| | | NO 160) | Chlamydomonas sphaeroides | | tgacaa- |
| | | | 5 H₂ gene coding sequence | | atg[csph5h2]₂₇ 3′ |
| 9.11 | Unique sequence i | (SEQ ID | First 30 bp of 5′ end of | | 5′ tatgcttgacaatcgtaatcctgg |
| | | NO 160) | Chlamydomonas surtseyiensis | | tgacaa- |
| | | | 5 H₂ gene coding sequence | | atg[csur5h2]₂₇ 3′ |
| 9.12 | Unique sequence i | (SEQ ID | First 30 bp of 5′ end of | | 5′ tatgcttgacaatcgtaatcctgg |
| | | NO 160) | Chlamydomonas ulvaensis 5 | | tgacaa- |
| | | | H₂ gene coding sequence | | atg[culv5h2]₂₇ 3′ |
| 9.13 | Unique sequence i | (SEQ ID | First 30 bp of 5′ end of | | 5′ tatgcttgacaatcgtaatcctgg |
| | | NO 160) | Chlamydomonas | | tgacaa- |
| | | | zimbabwiensis 5 H₂ gene | | atg[czim5h2]₂₇ 3′ |
| | | | coding sequence | | |
| 9.14 | Unique sequence i | (SEQ ID | First 30 bp of 5′ end of | | 5′ tatgcttgacaatcgtaatcctgg |
| | | NO 160) | Chlamydomonos reinhardtii 5 | | tgacaa- |
| | | | H₂ gene coding sequence | | atg[crei5h2]₂₇ 3′ |
| 10.1 | Last 30 bp of 5′ end of | | Unique sequence j | (SEQ ID | 5′ [cpul5h2]₃₀-taacaagaat |
| | Chlamydomonas | | | NO 161) | ctggctaatcaatcgatgca 3′ |
| | pulvinata 5 H₂ gene | | | | |
| | coding sequence | | | | |
| 10.2 | Last 30 bp of 3′ end of | | Unique sequence j | (SEQ ID | 5′ [cpyg5h2]₂₇taa- |
| | Chlamydomonas | | | NO 161) | taacaagaatctggctaatcaatcgat |
| | pygmaea 5 H₂ gene | | | | gca 3′ |
| | coding sequence | | | | |
| 10.3 | Last 30 bp of 3′ end of | | Unique sequence j | (SEQ ID | 5′ [crad5h2]₂₇taa- |
| | Chlamydomonas radiata | | | NO 161) | taacaagaatctggctaatcaatcgat |
| | 5 H₂ gene coding | | | | gca 3′ |
| | sequence | | | | |
| 10.4 | Last 30 bp of 3′ end of | | Unique sequence j | (SEQ ID | 5′ [crap5h2]₂₇taa- |
| | Chlamydomonas rapa 5 | | | NO 161) | taacaagaatctggctaatcaatcgat |
| | H₂ gene coding sequence | | | | gca 3′ |
| 10.5 | Last 30 bp of 3′ end of | | Unique sequence j | (SEQ ID | 5′ [csaj5h2]₂₇taa- |
| | Chlamydomonas sajao 5 | | | NO 161) | taacaagaatctggctaatcaatcgat |
| | H₂ gene coding sequence | | | | gca 3′ |
| 10.6 | Last 30 bp of 3′ end of | | Unique sequence j | (SEQ ID | 5′ [cseg²²²5h2]₂₇taa- |
| | Chlamydomonas | | | NO 161) | taacaagaatctggctaatcaatcgat |
| | segnis²²² 5 H₂ gene | | | | gca 3′ |
| | coding sequence | | | | |
| 10.7 | Last 30 bp of 3′ end of | | Unique sequence j | (SEQ ID | 5′ [cseg¹⁶³⁸5h2]₂₇taa- |
| | Chlamydomonas | | | NO 161) | taacaagaatctggctaatcaatcgat |
| | segnis¹⁶³⁸ 5 H₂ gene | | | | gca 3′ |
| | coding sequence | | | | |
| 10.8 | Last 30 bp of 3′ end of | | Unique sequence j | (SEQ ID | 5′ [cseg¹⁹¹⁹5h2]₂₇taa- |
| | Chlamydomonas | | | NO 161) | taacaagaatctggctaatcaatcgat |
| | segnis¹⁹¹⁹ 5 H₂ gene | | | | gca 3′ |
| | coding sequence | | | | |
| 10.9 | Last 30 bp of 3′ end of | | Unique sequence j | (SEQ ID | 5′ [csmi5h2]₂₇taa- |
| | Chlamydomonas smithii | | | NO 161) | taacaagaatctggctaatcaatcgat |
| | 5 H₂ gene coding | | | | gca 3′ |
| | sequence | | | | |
| 10.10 | Last 30 bp of 3′ end of | | Unique sequence j | (SEQ ID | 5′ [csph5h2]₂₇taa- |
| | Chlamydomonas | | | NO 161) | taacaagaatctggctaatcaatcgat |
| | sphaeroides 5 H₂ gene | | | | gca 3′ |
| | coding sequence | | | | |
| 10.11 | Last 30 bp of 3′ end of | | Unique sequence j | (SEQ ID | 5′ [csur5h2]₂₇taa- |
| | Chlamydomonas | | | NO 161) | taacaagaatctggctaatcaatcgat |
| | surtseyiensis 5 H₂ gene | | | | gca 3′ |
| | coding sequence | | | | |
| 10.12 | Last 30 bp of 3′ end of | | Unique sequence j | (SEQ ID | 5′ [culv5h2]₂₇taa- |
| | Chlamydomonas | | | NO 161) | taacaagaatctggctaatcaatcgat |
| | ulvaensis 5 H₂ gene | | | | gca 3′ |
| | coding sequence | | | | |
| 10.13 | Last 30 bp of 3′ end of | | Unique sequence j | (SEQ ID | 5′ [czim5h2]₂₇taa- |
| | Chlamydomonas | | | NO 161) | taacaagaatctggctaatcaatcgat |
| | zimbabwiensis 5 H₂ gene | | | | gca 3′ |
| | coding sequence | | | | |
| 10.14 | Last 30 bp of 3′ end of | | Unique sequence j | (SEQ ID | 5′ [crei5h2]₂₇taa- |
| | Chlamydomonas | | | NO 161) | taacaagaatctggctaatcaatcgat |
| | reinhardtii 5 H₂ gene | | | | gca 3′ |
| | coding sequence | | | | |

TABLE 3

| | | | | | |
|---|---|---|---|---|---|
| | | 5′ primer | | | |
| Product | 5′ primer | sequence | 3′ primer | 3′ primer sequence | Template |

| | | | | | | |
|---|---|---|---|---|---|---|
| Nonshuffled | Unique | | 5′ atccgtagtt | Complement | 5′ tcaggtccagaag | SEQ ID NO 148 |
| segment IX | sequence a- | | atccttatgg | of unique | ctgttaatcgatgcac | |
| | First 24 | | ccatcttagc | sequence b- | gtaacgaaatgag | |
| | nucleotides | | gcagttgggtca | complement | tctcgcccgcggc 3′ | |
| | of promoter | | ggggctggcgac 3′ | of last 25 base | | |
| | fragment of | | | pairs of the | | |
| | the lhcb1 | | | promoter | | |
| | gene | | | fragment of | | |
| | | | | the lhcb1 gene | | |
| Nonshuffled | Unique | | 5′ ttaaacgtcg | Complement | 5′ ttgtaagatctga | SEQ ID NO 151 |
| segment X | sequence c- | | tacgtccaag | of unique | atagcatgtatcagat | |
| | first 25 | | tataactaag | sequence d- | ttaacgaaatgag | |
| | nucleotides | | ccgacgtcgaccca | complement | tctcgcccgcggc 3′ | |
| | of 3′ UTR | | ctctagaggat 3′ | of last 25 base | | |
| | from | | | pairs of the | | |
| | RBCS2 gene | | | promoter | | |
| | | | | fragment of | | |
| | | | | the lhcb1 gene | | |
| Nonshuffled | Unique | | 5′ tcttccatcg | Complement | 5′ cttgaatgcctcg | SEQ ID NO 151 |
| segment XI | sequence e- | | taaatctagc | of unique | actagattattacaga | |
| | first 25 | | atcgattagc | sequence f- | ttaacgaaatgag | |
| | nucleotides | | ccgacgtcgaccca | complement | tctcgcccgcggc 3′ | |
| | of 3′ UTR | | ctctagaggat 3′ | of last 25 base | | |
| | from | | | pairs of the | | |
| | RBCS2 gene | | | promoter | | |
| | | | | fragment of | | |
| | | | | the lhcb1 gene | | |
| Nonshuffled | Unique | (SEQ ID NO 32) | 5′ atctgtaataatc | Complement | 5′ tcacacgattgtt | SEQ ID NO 179 |
| segment XII | sequence f- | | tagtcgaggcattcaa | of unique | aacgatttaagccagt | |
| | first 25 | | gatggccaagggcga | sequence g- | tttacttgtacagctc | |
| | nucleotides | | ggagctgttca 3′ | complement | gtccatgccg 3′ | |
| | of synthetic | | | of last 25 | | |
| | green | | | nucleotides of | | |
| | fluorescent | | | synthetic | | |
| | protein gene | | | green | | |
| | | | | fluorescent | | |
| | | | | protein gene | | |
| Nonshuffled | Unique | | 5′ aactggctta | Complement | 5′ tcgcacggtaatc | SEQ ID NO 150 |
| segment XIII | sequence g- | | aatcgttaac | of unique | gacagttatgttaaat | |
| | first 25 | | aatcgtgtga | sequence h- | ccaaatacgcccagcc | |
| | nucleotides | | ccgacgtcgaccca | Complement | cgcccatgga 3′ | |
| | of 3′ UTR | | ctctagaggat 3′ | of last 24 | | |
| | from | | | nucleotides of | | |
| | RBCS2 gene | | | 3′ UTR from | | |
| | | | | RBCS2 gene | | |

TABLE 4

| | | | |
|---|---|---|---|
| | 5′ end | | |
| Oligo # | corresponding to: | 3′ end corresponding to: | Sequence |

| | | | |
|---|---|---|---|
| 11.1 | Unique sequence b | First 25 nucleotides of | 5′ cgtgcatcgattaacagcttctggacctga |
| | | Chlamydomonas reinhardtii | atgtcggcgctcgtgctgaagccct 3′ |
| | | hydrogenase | |
| 11.2 | Unique sequence b | First 25 nucleotides of Clostriduim | 5′ cgtgcatcgattaacagcttctggacctga |
| | | pasteuranum hydrogenase | atgaaaacaataattataaatggtg 3′ |
| 11.3 | Unique sequence b | First 25 nucleotides of | 5′ cgtgcatcgattaacagcttctggacctga |
| | | Desulfovibrio vulgaris | atgagccgtaccgtcatggagcgca 3′ |
| | | hydrogenase | |
| 11.4 | Unique sequence b | First 25 nucleotides of Entamoeba | 5′ cgtgcatcgattaacagcttctggacctga |
| | | histolytica hydrogenase | atgccacctaaaccatcacatacac 3′ |
| 11.5 | Unique sequence b | First 25 nucleotides of | 5′ cgtgcatcgattaacagcttctggacctga |
| | | Scenedesmus obliquus | atgcctgagtggcaaccgggaggtc 3′ |
| | | hydrogenase | |
| 11.6 | Unique sequence b | First 25 nucleotides of Chlorella | 5′ cgtgcatcgattaacagcttctggacctga |
| | | fusca hydrogenase | atgtgttgccccgtggttgcaagta 3′ |
| 12.1 | Complement of | Complement of last 25 nucleotides | 5′ cttagttatacttggacgtacgacgtttaa |
| | unique sequence c | of Chlamydomonas reinhardtii | tcacttcttctcgtccttctcctcc 3′ |
| | | hydrogenase | |
| 12.2 | Complement of | Complement of last 25 nucleotides | 5′ cttagttatacttggacgtacgacgtttaa |
| | unique sequence c | of Clostriduim pasteuranum | ttattttttatatttaaagtgtaat 3′ |
| | | hydrogenase | |
| 12.3 | Complement of | Complement of last 25 nucleotides | 5′ cttagttatacttggacgtacgacgtttaa |
| | unique sequence c | of Desulfovibrio vulgaris | ctatgccttgttggcgctcgccatg 3′ |
| | | hydrogenase | |
| 12.4 | Complement of | Complement of last 25 nucleotides | 5′ cttagttatacttggacgtacgacgtttaa |
| | unique sequence c | of Entamoeba histolytica | ttagttttgatatctgggagtaaaa 3′ |
| | | hydrogenase | |
| 12.5 | Complement of | Complement of last 25 nucleotides | 5′ cttagttatacttggacgtacgacgtttaa |
| | unique sequence c | of Scenedesmus obliquus | tcacttctcatcgggcacgccgccg 3′ |
| | | hydrogenase | |
| 12.6 | Complement of | Complement of last 25 nucleotides | 5′ cttagttatacttggacgtacgacgtttaa |
| | unique sequence c | of Chlorella fusca hydrogenase | tcacttctcctctggaattccacct 3′ |
| 14 | Unique sequence d | First 25 nucleotides of | 5′ aatctgatacatgctattcagatcttacaa |
| | | Chlamydomonas reinhardtii | atggccatggctatgcgctccacct 3′ |
| | | ferredoxin | |
| 15 | Complement of | Complement of last 25 nucleotides | 5′ gctaatcgatgctagatttacgatggaaga |
| | unique sequence e | of Chlamydomonas reinhardtii | ttagtacagggcctcctcctggtgg 3′ |
| | | ferredoxin | |

U.S. Patents Referenced

Other patents included in paragraph [073] are U.S. Pat. Nos. 5,537,776; 5,965,408; 6,171,820; 6,174,673; 6,238,884; 6,326,204; 6,344,328; 6,352,842; 6,358,709; 6,361,97; 6,368,798; 6,440,668; 6,537,776; and 6,605,449.

Other patents referenced in this application are U.S. Pat. Nos. 5,871,952, 5,605,79, 5,830,721, 6,165,793, 6,180,406, 5,939,250, 6,171,820, 6,361,974, 6,358,709, 6,352,842, 6,238,884 6,420,175, 6,287,861, 6,277,589, 4,532,210 and WO 01/48185 (Fischer).

1. A method for engineering a cell to produce an increased amount of hydrogen comprising:
(a) providing a mutagenized nucleic acid sequence derived from a first gene that encodes a protein involved in a hydrogen production pathway;
(b) transforming a cell with said mutagenized nucleic acid sequence; and
(c) screening or selecting the cell for an increased amount of hydrogen.

2. The method of claim 1, wherein a plurality of mutagenized nucleic acid sequences are used to transform a population of cells, followed by the screening or selecting.

3. The method of claim 1, wherein the first gene is selected from the group that encodes ferredoxin, catalase, isoamylase, malate dehydrogenase, 14-3-3 protein, enolase, aldolase, ribosomal protein S8, ribosomal protein L17, ribosomal protein S18, ribosomal protein L37, ribosomal protein L12, ribosomal protein S15, iron-hydrogenase, nickel-iron hydrogenase, and components of the photosystem I, photosystem II, light harvesting antenna and cytochrome b₆-f complexes.

4. The method of claim 3, wherein the first gene encodes an iron-hydrogenase.

5. The method of claim 4, wherein at least one amino acid from the segment X¹X²X³X⁴X⁵X⁶GGVMEAAX⁷R or the segment ADX⁸TIX⁹EE is substituted by a different amino acid in the protein encoded by the first gene to generate the mutagenized nucleic acid sequence.

6. The method of claim 5, wherein the mutagenized nucleic acid sequence is generated by gene reassembly.

7. The method of claim 5, wherein the mutagenized nucleic acid sequence is generated by site-directed mutagenesis.

8. The method of claim 5, wherein an amino acid that is substituted for the at least one amino acid has a side chain of higher molecular weight than the side chain of the at least one amino acid.

9. The method of claim 5, wherein saturation mutagenesis is performed on the at least one amino acid.

10. The method of claim 5, wherein the mutagenized nucleic acid sequence is generated by a mutagenesis method described in U.S. Patents selected from the group consisting of 5,537,776; 5,965,408; 6,171,820; 6,174,673; 6,238,884; 6,326,204; 6,344,328; 6,352,842; 6,358,709; 6,361,97; 6,368,798; 6,440,668; 6,537,776; and 6,605,449.

11. The method of claim 6, wherein the gene reassembly is performed using nucleic acid molecules that encode proteins of SEQ ID NOs: 1-112 or segments thereof.

12. The method of claim 4, wherein the mutagenized nucleic acid sequence encodes an iron hydrogenase protein that functionally interacts with a ferredoxin protein in the cell.

13. The method of claim 1, wherein the screening or selecting occurs in the presence of oxygen at a concentration selected from the ranges comprising more than 0.5%, more than 5.0%, more than 10%, more than 15%, approximately 21%, more than 21%, more than 25%, more than 30% or more than 35% oxygen.

14. The method of claim 1, wherein the mutagenized nucleic acid sequence is operably linked to a promoter that is activated by light.

15. The method of claim 1, wherein the mutagenized nucleic acid sequence is generated by gene reassembly.

16. The method of claim 1, wherein the cell is a green algae species.

17. The method of claim 1, wherein cell is of the genus Chlamydomonas.

18. The method of claim 1, further comprising the steps of,
(a) identifying a first independent transformant which produces an increased amount of hydrogen from step (c) of claim 1;
(b) recovering the mutagenized nucleic acid sequence from the independent transformant;
(c) further mutagenizing the recovered mutagenized nucleic acid sequence to create a new library of mutagenized nucleic acid sequences;
(d) transforming cells with the new library of mutagenized nucleic acid sequences; and
(e) screening or selecting for a new independent transformant from the new library that generates an increased amount of hydrogen compared to the first independent transformant.

19. The method of claim 18 wherein the mutagenized nucleic acid sequencs are generated by gene reassembly.

20. The method of claim 18, wherein a plurality of mutagenized nucleic acid sequences are recovered from a plurality of independent transformants which produce an increased amount of hydrogen from step (c) of claim 1, and wherein the plurality of mutagenized nucleic acid sequences are subjected to gene reassembly to generate the new library.

21. The method of claim 1, wherein the screening or selecting occurs by culturing cells in liquid growth media.

22. The method of claim 21, wherein the growth media is a photoautotrophic growth-requiring minimal media.

23. The method of claim 1, wherein the screening or selecting occurs in a non-transparent culture container.

24. A method according to claim 1, wherein the mutagenized nucleic acid sequence is operably linked to a promoter that is constitutively activated.

25. The method of claim 15, wherein the mutagenized nucleic acid sequence is obtained by subjecting nucleic acid sequences that encode proteins that are expressed when cells are exposed to conditions more conducive to the generation of hydrogen to gene reassembly, wherein the proteins are naturally encoded by genes in organisms from more than one species.

26. The method of claim 19, wherein the proteins are iron hydrogenases or nickel-iron hydrogenases.

27. The method of claim 1, further comprising repeating the steps of claim 1 using a second gene distinct from the first gene.

28. The method of claim 27, further comprising:
(a) mating at least one cell of a strain containing a mutagenized form of the first gene:
i. wherein the at least one cell is identified by the screening or selecting; or
ii. wherein the at least one cell is derived through mating from a cell identified by the screening or selecting;
to at least one cell of a distinct strain containing a mutagenized form of the second gene:
iii. wherein the at least one cell is identified by the screening or selecting; or
iv. wherein the at least one cell is derived through mating from a cell identified by the screening or selecting; and
(b) screening or selecting for a progeny cell that produces an increased amount of hydrogen compared to any parent cell.

29. A method of hydrogen production, comprising:
(a) placing cell containing a mutagenized nucleic acid sequence corresponding to a gene that is involved in a hydrogen production pathway into liquid culture media or on to solid culture media, wherein the mutagenized nucleic acid sequence is operably linked to a transcriptional promoter sequence;
(b) culturing said transformed cell under conditions sufficient to stimulate transcription of said mutagenized nucleic acid sequence(s); and
(c) collecting an evolved gas.

30. The method of claim 29, wherein the culture media is photoautotrophic growth requiring media.

31. A method of multiparental mating of microbes that mate in response to a stimulus, comprising:
(a) providing a cell from each of 3 or more strains of microbes capable of mating to each other in culture medium;
(b) providing the stimulus;
(c) allowing cells to mate and produce progeny;
(d) allowing the progeny cells to achieve sexual reproduction capability;
(e) providing the stimulus at least one more time; and
(f) screening or selecting the further progeny for a desired phenotype.

32. The method of claim 31, wherein the microbes are green algae and the stimulus is the removal of nitrogen from the media and illumination by light comprising a wavelength between about 0.42-0.52 micrometers.

33. The method of claim 32, wherein the green algae are of the Chlamydomonas genus.

34. The method of claim 33, wherein the species is selected from the group comprising reinhardtii, eugametos, incerta, and moewusii.

35. The method of claim 31, wherein the stimulus is interruption of exponential growth in continuous light with a reduction in light, followed by addition of light.

36. The method of claim 35, wherein the reduction in light occurs for a period selected from the group consisting of at least 1, 2, 3, 4, 5, 6, 7,8,9, 10, 11, 12, or more than 12 hours.

36. The method of claim 31, wherein the microbes are of the Scendesmus genus and the stimulus is the addition of chromium to the culture media.

37. The method of claim 31, wherein the desired phenotype is hydrogen production.

38. The method of claim 31, wherein nucleic acid exchange occurs between only two parental cells at a time during the mating process.
